(19) United States
(12) Patent Application Publication
(10) Pub. No.: US 2024/0074986 A1
(43) Pub. Date: Mar. 7, 2024

(54) Methods of Enhancing Non-Viral Gene Therapy

(71) Applicant: Spark Therapeutics, Inc., Philadelphia, PA (US)

(72) Inventors: Ali Nahvi, Needham, MA (US); Xavier Anguela, Barcelona (ES); Rui Zhang, Winchester, MA (US)

(21) Appl. No.: 18/258,981

(22) PCT Filed: Dec. 22, 2021
(86) PCT No.: PCT/US2021/073084

§ 371 Date: Jun. 22, 2023

Related U.S. Application Data
(60) Provisional application No. 63/130,105, filed on Dec. 23, 2020.

(51) Int. Cl. A61K 9/51; A61K 31/573; A61K 31/663; A61K 48/00; C07K 16/28; C12N 15/88

(52) U.S. Cl. CPC A61K 9/5123; A61K 31/573; A61K 31/663; A61K 48/0058; C07K 16/2866; C12N 15/88

(57) ABSTRACT

Methods of delivering a transgene to a subject in need thereof are described. In particular, the methods include administering to the subject (i) a phagocyte-depleting agent, and (ii) a pharmaceutical composition comprising a non-viral vector comprising the transgene and a pharmaceutically acceptable carrier. The methods can be used to treat a subject in need of treatment for a disease caused by a loss of function or activity of a protein, or to treat a subject in need of treatment for a disease caused by a gain of function activity or expression of a protein.

### RELATED APPLICATIONS

This application claims priority to U.S. Provisional Patent Application No. 63/130,105, filed Dec. 23, 2020, the entire contents of which are incorporated herein by reference, including all text, tables, and drawings.

### FIELD OF THE INVENTION

The invention relates to the field of gene therapy. In particular, it relates to methods of enhancing non-viral gene therapy.

### BACKGROUND OF THE INVENTION

Gene therapy involves delivery of foreign nucleic acids to cells to provide a therapeutic benefit. Genetic diseases for which a causative abnormality is known can potentially be treated using gene therapy, either by blocking a gene that is being either mis-or overexpressed, or by providing a working copy of a malfunctioning gene. Such gene therapy approaches are currently being developed to treat or cure a number of diseases that arise from genetic abnormalities.

An effective delivery system for the therapeutic genetic material is required for successful gene therapy. Successful delivery of the genetic material requires that, for example, the gene delivery system be deliverable to the subject, be able to protect the genetic material from enzymatic degradation, have a long lifetime in the body, be able to reach the site within the body where it is needed, be biocompatible and biodegradable or have tolerable toxicity, and be able to cross the cell membrane and transit through the cytosol and/or cross the nuclear membrane to release the genetic material at the desired point of action.

Gene therapy can be delivered by viral and non-viral vectors. Viral vectors demonstrate high transduction efficiency and exhibit stable long-term expression of a foreign gene when the recombinant DNA remains episomal or is integrated into the chromosome. However, their in vivo efficacy may be limited due to concerns of toxicity, random integration of genetic materials into the genome, disruption of proper gene function, cancer formation, potential replication, cell division-dependent dilution, limited DNA carrying capacity, severe immune and inflammatory responses due, in part, to humoral and cell-mediated immune responses to the vector capsid, and the inability to re-dose subjects due to the presence of pre-existing antibodies to the vector capsid in a subset of subjects.

Non-viral gene delivery approaches are being developed to overcome the deficiencies of viral vectors. Compared to viral vectors, certain types of non-viral vectors are easier to make, do not require replication, and are less likely to produce adaptive immune responses due to the lack of a protein-based vector capsid. In addition, non-viral vectors offer some invaluable advantages, including the lack of transgene size restriction associated with viral vectors, the ability to dose subjects with pre-existing antibodies to vector capsid, and the ability to re-dose a subject. However, non-viral delivery approaches suffer from challenges such as toxicity, transfection efficiency, nucleic acid degradation, and innate immunity. In addition, non-viral approaches have demonstrated low efficiency of gene delivery to somatic targets and lower in vivo gene expression levels than viral approaches.

The liver is a central organ in metabolism, and it is a target for gene therapy of many inherited metabolic disease and disorders, including inborn errors of metabolism and hemophilia, as well as acquired diseases such as liver cancer and hepatitis. The particular cell targets for liver-directed gene therapies are the parenchymal liver cells, or hepatocytes. However, nanoparticles are predominantly taken up by non-parenchymal cells including Kupffer cells (˜50%) and liver sinusoidal endothelial cells (˜30%), limiting the efficacy of hepatocyte-directed gene therapy (Shi et al., J Histochem Cytochem. 2011 59(8):727-40; Jacobs et al., Pharmaceuticals 2012, 5(12):1372-1392). Examples of other tissue-specific macrophages, also referred to as resident macrophages, include intestinal macrophages in the gut, microglial cells in the brain, alveolar macrophages in the lung, resident kidney macrophages, skin macrophages, red pulp macrophages in the spleen, and osteoclasts in bone. Site-specific improved efficiency of gene therapy could be achieved through enhancing the entry of non-viral vectors into the target cells and reducing the uptake of the vectors by non-target cells.

There remains a need for methods of effectively, specifically, and safely delivering therapeutic genetic material to targets of interest.

### BRIEF SUMMARY OF THE INVENTION

Disclosed herein are methods for delivering a transgene to a subject in need thereof. Methods according to the instant invention can be used to treat a subject in need of treatment for a disease caused by a loss of function or activity of a protein, or to treat a subject in need of treatment for a disease caused by a gain of function activity or expression of a protein.

Although not wishing to be bound by any theory or particular mechanism, it is believed that depletion of phagocytic immune cells by administration of a phagocyte-depleting agent reduces the unproductive uptake of non-viral vectors by non-target cells, enabling enhanced entry of the non-viral vectors into target cells and resulting in improved transgene expression.

In certain embodiments, the invention relates to methods of delivering a transgene to a subject in need thereof comprising:
a. administering to the subject a phagocyte-depleting agent; and
b. administering to the subject a pharmaceutical composition comprising a non-viral vector comprising the transgene and a pharmaceutically acceptable carrier.

In certain embodiments, the pharmaceutical composition comprises:
a. a non-viral delivery nanoparticle; and
b. the non-viral vector, wherein the non-viral vector comprises the transgene operably linked to a promoter.

In certain embodiments, the phagocyte-depleting agent is a monocyte and/or macrophage-depleting agent.

In certain embodiments, the monocyte and/or macrophage-depleting agent is a CD115 inhibiting agent.

In certain embodiments, the CD115 inhibiting agent is an antibody or an antigen binding fragment thereof that specifically binds to CD115.

In certain embodiments, the anti-CD115 antibody or antigen binding fragment thereof is emactuzumab, AMG820, or cabiralizumab.

In certain embodiments, the CD115 inhibiting agent is a small molecule inhibitor of CD115.

In certain embodiments, the small molecule inhibitor of CD115 is pexidartinib.

In certain embodiments, the monocyte and/or macrophage-depleting agent is not clodronate, or the monocyte and/or macrophage-depleting agent comprises clodronate and one or more additional phagocyte-depleting agent such as a CD115 inhibiting agent or a CD177, CD14, CD15, CD11b, CD16, CD32, CD33, CD44, CD45, CD66b, CD18, or CD62L inhibiting agent.

In certain embodiments, the phagocyte-depleting agent is a neutrophil-depleting agent.

In certain embodiments, the neutrophil-depleting agent is a CD177, CD14, CD15, CD11b, CD16, CD32, CD33, CD44, CD45, CD66b, CD18, or CD62L inhibiting agent, or an agent that inhibits the corresponding or equivalent human protein and/or functional cell type.

In certain embodiments, the CD177, CD14, CD15, CD11b, CD16, CD32, CD33, CD44, CD45, CD66b, CD18, or CD62L inhibiting agent is an antibody or an antigen binding fragment thereof that specifically binds to CD177, CD14, CD15, CD11b, CD16, CD32, CD33, CD44, CD45, CD66b, CD18, or CD62L.

In certain embodiments, the CD177, CD14, CD15, CD11b, CD16, CD32, CD33, CD44, CD45, CD66b, CD18, or CD62L inhibiting agent is a small molecule inhibitor of CD177, CD14, CD15, CD11b, CD16, CD32, CD33, CD44, CD45, CD66b, CD18, or CD62L.

In certain embodiments, the phagocyte-depleting agent is a dendritic cell-depleting agent.

In certain embodiments, the non-viral vector and phagocyte-depleting agent are co-administered.

In certain embodiments, the phagocyte-depleting agent is administered at least one day before the non-viral vector is administered, optionally no more than 1 year before the non-viral vector is administered.

In certain embodiments, the method further comprises administering to the subject a bisphosphonate, preferably together with one or more other phagocyte-depleting agents.

In certain embodiments, the bisphosphonate is clodronate, pamidronate, ibandronate, or zoledronate, optionally clodronate.

In certain embodiments, the method further comprises administering to the subject an immunosuppressant.

In certain embodiments, the immunosuppressant is a steroid.

In certain embodiments, the steroid is a corticosteroid, optionally dexamethasone.

In certain embodiments, the method results in expression of the transgene in the subject.

In certain embodiments, the subject has a minimal or absent undesirable immune response induced by the non-viral vector.

In certain embodiments, the non-viral delivery particle is selected from the group consisting of a lipid nanoparticle, a polymer nanoparticle, a protein-based nanoparticle, and a peptide cage.

In certain embodiments, the non-viral vector is a dsDNA molecule, wherein the dsDNA molecule is selected from the group consisting of a minicircle, a plasmid, an open linear duplex DNA, and a closed-ended linear duplex DNA (CELiD/ceDNA/doggybone DNA).

In certain embodiments, the non-viral vector is an ssDNA molecule, wherein the ssDNA molecule is a closed circular or an open linear DNA.

In certain embodiments, the transgene encodes a therapeutic or prophylactic protein or peptide.

In certain embodiments, the transgene encodes a therapeutic or prophylactic nucleic acid.

Also disclosed herein are combinations or compositions, for example and without limitation, packages and kits, having components that can be used to practice methods according to the instant invention. In certain embodiments, a package or kit has disposed therein: (a) a pharmaceutical composition comprising a non-viral vector comprising a transgene and a pharmaceutically acceptable carrier; (b) a phagocyte-depleting agent; and (c) a label with instructions for performing a method as disclosed herein. In certain embodiments, (a) and (b) are in separate or the same container. In certain embodiments, the combination, package or kit can further comprise an immunosuppressant.

### BRIEF DESCRIPTION OF THE DRAWINGS

The foregoing summary, as well as the following detailed description of the invention, will be better understood when read in conjunction with the appended drawings. It should be understood that the invention is not limited to the precise embodiments shown in the drawings. In the drawings:

FIG. 1 shows the immune response results of the study described in Example 1, specifically, FIG. 1A is a graph showing levels of IFN-γ cytokine production in the plasma of animals 6 hours following dosing with 50 μg of DNA-LNP, with or without anti-CD115 or anti-Ly6G antibodies or large ODN or H-151; and FIG. 1B is a graph showing levels of IFN-γ cytokine production in the plasma of animals 6 hours following dosing with 10 μg of DNA-LNP, with or without anti-CD115 or anti-Ly6G antibodies or large ODN or H-151.

FIG. 2 shows the efficacy results of the study described in Example 1, specifically, FIG. 2A is a graph showing expression levels of the FIX transgene in the plasma of animals 1, 8, 14, 28, 63, 84, and 98 days following dosing with 50 μg of DNA-LNP, with or without anti-CD115 or anti-Ly6G antibodies or large ODN or H-151; and FIG. 2B is a graph showing expression levels of the FIX transgene in the plasma of animals 1, 8, 14, 28, 63, 84, and 98 days following dosing with 10 μg of DNA-LNP, with or without anti-CD115 or anti-Ly6G antibodies or large ODN or H-151.

FIG. 3 shows the immune response results of the study described in Example 2, specifically, FIG. 3A is a graph showing levels of IFN-γ cytokine production in the plasma of animals 6 hours following dosing with 50 μg of DNA-LNP, with or without anti-CD115 and/or clodronate; and FIG. 3B is a graph showing levels of IL-6 cytokine production in the plasma of animals 6 hours following dosing with 50 μg of DNA-LNP, with or without anti-CD115 and/or clodronate.

FIG. 4 shows the efficacy results of the study described in Example 2, specifically, FIG. 4 is a graph showing expression levels of the FIX transgene in the plasma of animals 1 week following dosing with 50 μg of DNA-LNP, with or without anti-CD115 and/or clodronate.

FIG. 5 shows depletion efficacy resulting from treatment with clodronate, anti-CD115, and pexidartinib. FIG. 5A provides results measuring CLEC4F+ cells. FIG. 5B provides results measuring CD68+ cells.

FIG. 6 shows depletion efficacy resulting from treatment with pexidartinib mixed with chow. FIG. 6A provides results measuring CD68+ cells. FIG. 6B provides results measuring CLEC4F+ cells.

FIG. 7 shows hFIX expression in mouse treated with pexidartinib and dosed with DNA-LNP-5. Mice were pretreated with pexidartinib for 7 or 21 days. hFIX levels in mouse plasma were quantified using ELISA 1, 2, 3 or 4 weeks after dosing.

FIG. 8 shows hFIX expression in mouse treated with anti-CD-115 antibodies and dosed with DNA-LNP-5. Mice were pretreated with anti-CD-115 antibodies three times (days −5, −3, and −1) before DNA-LNP-5 dosing. hFIX levels in mouse plasma were quantified using ELISA 1, 2, 4 or 12 weeks after dosing. In FIG. 8, “ns” denotes not significant and *P<0.05 by t-test; and LLOQ denotes lower limit of quantitation.

FIG. 9 shows hFIX expression in mouse treated with anti-CD-115 antibodies and dosed with DNA-LNP-3. Mice were pretreated with anti-CD-115 antibodies four times (days −10, −8, −6, and −2) before DNA-LNP-3 dosing. hFIX levels in mouse plasma were quantified using ELISA 1, 2, 4 or 12 weeks after dosing. In FIG. 9, “ns” denotes not significant, *P<0.05, and **P<0.01 by t-test; and LLOQ denotes lower limit of quantitation.

FIG. 10 shows hFIX expression in mouse treated with anti-CD-115 antibodies and dosed with DNA-LNP-4. Mice were pretreated with anti-CD-115 antibodies four times (days −10, −8, −6, and −2) before DNA-LNP-4 dosing. hFIX levels in mouse plasma were quantified using ELISA 1, 2, 4 or 12 weeks after dosing. In FIG. 10, “ns” denotes not significant, *P<0.05, and ***P<0.001 by t-test; and LLOQ denotes lower limit of quantitation.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning commonly understood to one of ordinary skill in the art to which this invention pertains. Otherwise, certain terms cited herein have the meanings as set in the specification. All patents, published patent applications and publications cited herein are incorporated by reference as if set forth fully herein.

It must be noted that as used herein and in the appended claims, the singular forms “a,” “an,” and “the” include plural reference unless the context clearly dictates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising”, “containing”, “including”, and “having”, whenever used herein in the context of an aspect or embodiment of the invention can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

All of the features disclosed herein can be combined in any combination. Each feature disclosed in the specification can be replaced by an alternative feature serving a same, equivalent, or similar purpose. Thus, unless expressly stated otherwise, disclosed features are an example of a genus of equivalent or similar features.

The term “about” as used herein refers to a value within 10% of the underlying parameter (i.e., plus or minus 10%). For example, “about 1:10” means 1.1:10.1 or 0.9:9.9, and about 5 hours means 4.5 hours or 5.5 hours, etc. The term “about” at the beginning of a string of values modifies each of the values by 10%.

All numerical values or numerical ranges include integers within such ranges and fractions of the values or the integers within ranges unless the context clearly indicates otherwise. Thus, to illustrate, reference to reduction of 95% or more includes 95%, 96%, 97%, 98%, 99%, 100% etc., as well as 95.1%, 95.2%, 95.3%, 95.4%, 95.5%, etc., 96.1%, 96.2%, 96.3%, 96.4%, 96.5%, etc., and so forth. Thus, to also illustrate, reference to a numerical range, such as “1-4” includes 2, 3, as well as 1.1, 1.2, 1.3, 1.4, etc., and so forth. For example, “1 to 4 weeks” includes 7, 8, 9, 10, 11, 12, 13, 14, 15, 16, 17, 18, 19, 20, 21, 22, 23, 24, 25, 26, 27, or 28 days.

Further, reference to a numerical range, such as “0.01 to 10” includes 0.011, 0.012, 0.013, etc., as well as 9.5, 9.6, 9.7, 9.8, 9.9, etc., and so forth. For example, a dosage of about “0.01 mg/kg to about 10 mg/kg” body weight of a subject includes 0.011 mg/kg, 0.012 mg/kg, 0.013 mg/kg, 0.014 mg/kg, 0.015 mg/kg etc., as well as 9.5 mg/kg, 9.6 mg/kg, 9.7 mg/kg, 9.8 mg/kg, 9.9 mg/kg etc., and so forth.

Reference to an integer with more (greater) or less than includes any number greater or less than the reference number, respectively. Thus, for example, reference to more than 2 includes 3, 4, 5, 6, 7, 8, 9, 10, 11, 12, 13, 14, 15, etc., and so forth. For example, administration of a non-viral vector and/or a phagocyte-depleting agent “two or more” times includes 3, 4, 5, 6, 7, 8, 9, 10, 11, 12, 13, 14, 15, or more times.

Further, reference to a numerical range, such as “1 to 90” includes 1.1, 1.2, 1.3, 1.4, 1.5, etc., as well as 81, 82, 83, 84, 85, etc., and so forth. For example, “between about 1 minute to about 90 days” includes 1.1 minutes, 1.2 minutes, 1.3 minutes, 1.4 minutes, 1.5 minutes, etc., as well as one day, 2 days, 3 days, 4 days, 5 days . . . 81 days, 82 days, 83 days, 84 days, 85 days, etc., and so forth.

In an attempt to help the reader of the application, the description has been separated into various paragraphs or sections, or is directed to certain embodiments of the application. These separations should not be considered as disconnecting the substance of a paragraph or section or embodiments from the substance of another paragraph or section or embodiments. To the contrary, one skilled in the art will understand that the description has broad application and encompasses all the combinations of the various sections, paragraphs and sentences that can be contemplated. The discussion of any embodiment is meant only to be exemplary and is not intended to suggest that the scope of the disclosure, including the claims, is limited to these examples.

Provided herein are methods to improve gene therapy comprising administration of an agent that modulates immune cells. Methods according to the instant invention can be used to treat a subject in need of treatment for a disease, such as one caused by a loss of function or activity of a protein, or to treat a subject in need of treatment for a disease caused by a gain of function activity or expression of a protein.

In a general aspect, provided herein are methods of delivering a transgene to a subject in need thereof comprising:
a. administering to the subject a phagocyte-depleting agent; and
b. administering to the subject a pharmaceutical composition comprising a non-viral vector comprising the transgene and a pharmaceutically acceptable carrier.

In certain embodiments, the pharmaceutical composition comprises:
a. a non-viral delivery nanoparticle; and
b. a non-viral vector, wherein the non-viral vector comprises the transgene operably linked to a promoter.

### Gene Transfer Systems

The term “vector” or “gene transfer vector” as used herein, refers to a nucleic acid molecule comprising a gene of interest. Examples of vectors include, but are not limited to, viral vectors delivered by viral particles or virus-like particles (VLPs) that resemble viral particles but are non-infectious, such as retroviral, adenoviral, adeno-associated viral, and lentiviral particles or VLPs; and non-viral vectors delivered by non-viral gene transfer systems, such as microinjection, electroporation, liposomes, large natural polymers, large synthetic polymers, and polymers comprised of both natural and synthetic components.

As used herein, a “non-viral vector” refers to a vector that is not delivered by viral particles or by viral-like particles (VLPs). According to certain embodiments, a non-viral vector is a vector that is not delivered by a capsid. The vector can be encapsulated, admixed, or otherwise associated with the non-viral delivery nanoparticle.

As used herein, the term “gene transfer system” refers to any means of delivering a composition comprising a nucleic acid sequence (e.g., transgene) to a cell or tissue. For example, a gene transfer system can be a viral gene transfer system, e.g., intact viruses, modified viruses and VLPs to facilitate delivery of a viral vector to a desired cell or tissue. A gene transfer system can also be a non-viral delivery system that does not comprise viral coat protein or form a viral particle or VLP, e.g., liposome-based systems, polymer-based systems, protein-based systems, metallic particle-based systems, peptide cage systems, etc.

Any suitable non-viral delivery system known to those skilled in the art in view of the present disclosure can be used in the invention. The non-viral delivery nanoparticle can be, for example, a lipid-based nanoparticle, a polymer-based nanoparticle, a protein-based nanoparticle, a microparticle, a microcapsule, a metallic particle-based nanoparticle, a peptide cage nanoparticle, etc.

A non-viral delivery nanoparticle of the instant invention can be constructed by any method known in the art, and a non-viral vector of the instant invention comprising the non-viral delivery nanoparticle and nucleic acid molecule comprising a therapeutic transgene can be constructed by any method known in the art.

### Lipid-Based Delivery Systems

Lipid-based delivery systems are well known in the art, and any suitable lipid-based delivery system known to those skilled in the art in view of the present disclosure can be used in the invention. Examples of lipid-based delivery systems include, e.g., liposomes, lipid nanoparticles, micelles, or extracellular vesicles.

A “lipid nanoparticle” or “LNP” refers to a lipid-based vesicle useful for delivery of nucleic acid molecules and having dimensions on the nanoscale, i.e., from about 10 nm to about 1000 nm, or from about 50 nm to about 500 nm, or from about 50 nm to about 200 nm.

DNA is negatively charged. Thus, it can be beneficial for the LNP to comprise a cationic lipid such as, for example, an amino lipid. Exemplary amino lipids have been described in U.S. Pat. Nos. 9,352,042, 9,220,683, 9,186,325, 9,139,554, 9,126,966 9,018,187, 8,999,351, 8,722,082, 8,642,076, 8,569,256, 8,466,122, and 7,745,651 and U.S. Patent Publication Nos. 2016/0213785, 2016/0199485, 2015/0265708, 2014/0288146, 2013/0123338, 2013/0116307, 2013/0064894, 2012/0172411, and 2010/0117125, all of which are incorporated herein in their entirety. In certain embodiments, the LNP comprises amino lipids such as any of those described in WO2013/063468, incorporated herein in its entirety.

The terms “cationic lipid” and “amino lipid” are used interchangeably herein to include those lipids and salts thereof having one, two, three, or more fatty acid or fatty alkyl chains and a pH-titratable amino group (e.g., an alkylamino or dialkylamino group). The cationic lipid is typically protonated (i.e., positively charged) at a pH below the pKa of the cationic lipid and is substantially neutral at a pH above the pKa. The cationic lipids can also be titratable cationic lipids. In certain embodiments, the cationic lipids comprise: a protonatable tertiary amine (e.g., pH-titratable) group; C18 alkyl chains, wherein each alkyl chain independently has 0 to 3 (e.g., 0, 1, 2, or 3) double bonds; and ether, ester, or ketal linkages between the head group and alkyl chains.

Cationic lipids can include, without limitation, 1,2-dilinoleyloxy-N,N-dimethylaminopropane (DLinDMA), 1,2-dilinolenyloxy-N,N-dimethylaminopropane (DLenDMA), 1,2-di-γ-linolenyloxy-N,N-dimethylaminopropane (γ-DLenDMA), 2,2-dilinoleyl-4-(2-dimethylaminoethyl)-[1,3]-dioxolane (DLin-K-C2-DMA, also known as DLin-C2K-DMA, XTC2, and C2K), 2,2-dilinoleyl-4-dimethylaminomethyl[1,3]-dioxolane (DLin-K-DMA), dilinoleylmethyl-3-dimethylaminopropionate (DLin-M-C2-DMA, also known as MC2), (6Z,9Z,28Z,31 Z)-heptatriaconta-6,9,28,31-tetraen-19-yl 4-(dimethylamino)butanoate (DLin-M-C3-DMA, also known as MC3), salts thereof, and mixtures thereof. Other cationic lipids also include, but are not limited to, 1,2-distearyloxy-N,N-dimethyl-3-aminopropane (DSDMA), 1,2-dioleyloxy-N,N-dimethyl-3-aminopropane (DODMA), 2,2-dilinoleyl-4-(3-dimethylaminopropyl)-[1,3]-dioxolane (DLin-K-C3-DMA), 2,2-dilinoleyl-4-(3-dimethylaminobutyl)-[1,3]-dioxolane (DLin-K-C4-DMA), DLen-C2K-DMA, γ-DLen-C2K-DMA, and (DLin-MP-DMA) (also known as 1-B11).

Still further cationic lipids can include, without limitation, 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ), 1,2-dilinoleylcarbamoyloxy-3-dimethylaminopropane (DLin-C-DAP), 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanedio (DOAP), 1,2-dilinoleyloxo-3-(2-N,N-dimethylamino)ethoxypropane (DLin-EG-DMA), N,N-dioleyl-N,N-dimethylammonium chloride (DODAC), N-(1-(2,3-dioleyloxy)propyl)-N,N,N-trimethylammonium chloride (DOTMA), N,N-distearyl-N,N-dimethylammonium bromide (DDAB), N-(1-(2,3-dioleoyloxy)propyl)-N,N,N-trimethylammonium chloride (DOTAP), 3-(N-(N′,N′-dimethylaminoethane)-carbamoyl)cholesterol (DC-Chol), N-(1,2-dimyristyloxyprop-3-yl)-N,N-dimethyl-N-hydroxyethyl ammonium bromide (DMRIE), 2,3-dioleyloxy-N-[2(spermine-carboxamido)ethyl]-N,N-dimethyl-1-propanaminiumtrifluoroacetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethyl-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-N,N′-dilinoleylcarbamyl-3-dimethylaminopropane (DLincarbDAP), dexamethasone-sperimine (DS) and disubstituted spermine (D2S) or mixtures thereof.

A number of commercial preparations of cationic lipids can be used, such as, LIPOFECTIN® (including DOTMA and DOPE, available from GIBCO/BRL), and LIPOFECTAMINE® (comprising DOSPA and DOPE, available from GIBCO/BRL).

Other commercially available ionizable lipids that can be used include, e.g., SS-OP (NOF American Corporation), C12-200 (described in Kauffman et al., Nano Lett. 2015, 15, 11, 7300-7306, hereby incorporated by reference herein), 306Oi10, CKK-E12, MC3, Branched-CKK-E12, Lipid 5, Lipid 9, ATX-002 and ATX-003 (described in Payne and Chivukula, International Publication No. WO2015074085, hereby incorporated by reference herein), SM-102 (Cayman Chemicals); ALC-0315 (Selleck Chemicals) and Merck-32.

In certain embodiments, cationic lipid can be present in an amount from about 10% by molar ratio of the LNP to about 85% by molar ratio of the lipid nanoparticle, or from about 50% by molar ratio of the LNP to about 75% by molar ratio of the LNP.

Sterols can confer fluidity to the LNP. As used herein, “sterol” refers to any naturally occurring sterol of plant (phytosterols) or animal (zoosterols) origin as well as non-naturally occurring synthetic sterols, all of which are characterized by the presence of a hydroxyl group at the 3-position of the steroid A-ring. The sterol can be any sterol conventionally used in the field of liposome, lipid vesicle or lipid particle preparation, most commonly cholesterol. Phytosterols can include campesterol, sitosterol, and stigmasterol. Sterols also include sterol-modified lipids, such as those described in U.S. Patent Application Publication 2011/0177156. In certain embodiments, a sterol can be present in an amount from about 5% by weight of the LNP to about 50% by weight of the lipid nanoparticle or from about 10% by weight of the LNP to about 25% by weight of the LNP.

LNP can comprise a neutral lipid. Neutral lipids can comprise any lipid species which exists either in an uncharged or neutral zwitterionic form at physiological pH. Such lipids include, without limitation, diacylphosphatidylcholine, diacylphosphatidylethanolamine, ceramide, sphingomyelin, dihydrosphingomyelin, cephalin, and cerebrosides. The selection of neutral lipids is generally guided by consideration of, inter alia, particle size and the requisite stability. In certain embodiments, the neutral lipid component can be a lipid having two acyl groups (e.g., diacylphosphatidylcholine and diacylphosphatidylethanolamine).

Lipids having a variety of acyl chain groups of varying chain length and degree of saturation are available or can be isolated or synthesized by well-known techniques. In certain embodiments, lipids containing saturated fatty acids with carbon chain lengths in the range of C14 to C22 can be used. In certain embodiments, lipids with mono or diunsaturated fatty acids with carbon chain lengths in the range of C14 to C22 are used. Additionally, lipids having mixtures of saturated and unsaturated fatty acid chains can be used. Exemplary neutral lipids include, without limitation, 1,2-dioleoyl-sn-glycero-3-phosphatidyl-ethanolamine (DOPE), 1,2-distearoyl-sn-glycero-3-phosphocholine (DSPC), 1-palmitoyl-2-oleoyl-sn-glycero-3-phosphocholine (POPC), or any related phosphatidylcholine. The neutral lipids can also be composed of sphingomyelin, dihydrosphingomyelin, or phospholipids with other head groups, such as serine and inositol.

In certain embodiments, the neutral lipid can be present in an amount from about 0.1% by weight of the lipid nanoparticle to about 99% by weight of the LNP, or from about 5% by weight of the LNP to about 15% by weight of the LNP, e.g. about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99%.

LNP encapsulated nucleic acid molecules can be incorporated into pharmaceutical compositions, e.g., a pharmaceutically acceptable carrier or excipient. Such pharmaceutical compositions are useful for, among other things, administration and delivery of LNP encapsulated nucleic acid molecules to a subject in vivo or ex vivo.

Preparations of LNP can be combined with additional components, which can include, for example and without limitation, polyethylene glycol (PEG) and sterols.

The term “PEG” refers to a polyethylene glycol, a linear, water-soluble polymer of ethylene PEG repeating units with two terminal hydroxyl groups. PEGs are classified by their molecular weights; for example, PEG 2000 has an average molecular weight of about 2,000 daltons, and PEG 5000 has an average molecular weight of about 5,000 daltons. PEGs are commercially available from Sigma Chemical Co. and other companies and include, for example, the following functional PEGs: monomethoxypolyethylene glycol (MePEG-OH), monomethoxypolyethylene glycol-succinate (MePEG-S), monomethoxypolyethylene glycol-succinimidyl succinate (MePEG-S-NHS), monomethoxypolyethylene glycol-amine (MePEG-NH2), monomethoxypolyethylene glycol-tresylate (MePEG-TRES), and monomethoxypolyethylene glycol-imidazolyl-carbonyl (MePEG-IM).

In certain embodiments, PEG can be a polyethylene glycol with an average molecular weight of about 550 to about 10,000 daltons and is optionally substituted by alkyl, alkoxy, acyl or aryl. In certain embodiments, the PEG can be substituted with methyl at the terminal hydroxyl position. In certain embodiments, the PEG can have an average molecular weight from about 750 to about 5,000 daltons, or from about 1,000 to about 5,000 daltons, or from about 1,500 to about 3,000 daltons or from about 2,000 daltons or of about 750 daltons. The PEG can be optionally substituted with alkyl, alkoxy, acyl or aryl. In certain embodiments, the terminal hydroxyl group can be substituted with a methoxy or methyl group.

PEG-modified lipids include the PEG-dialkyloxypropyl conjugates (PEG-DAA) described in U.S. Pat. Nos. 8,936,942 and 7,803,397. PEG-modified lipids (or lipid-polyoxyethylene conjugates) that are useful can have a variety of “anchoring” lipid portions to secure the PEG portion to the surface of the lipid vesicle. Examples of suitable PEG-modified lipids include PEG-modified phosphatidylethanolamine and phosphatidic acid, PEG-ceramide conjugates (e.g., PEG-CerC14 or PEG-CerC20) which are described in U.S. Pat. No. 5,820,873, PEG-modified dialkylamines and PEG-modified 1,2-diacyloxypropan-3-amines. In certain embodiments, the PEG-modified lipid can be PEG-modified diacylglycerols and dialkylglycerols. In certain embodiments, the PEG can be in an amount from about 0.5% by weight of the LNP to about 20% by weight of the LNP, or from about 5% by weight of the LNP to about 15% by weight of the LNP.

Furthermore, LNP can be a PEG-modified and/or a sterol-modified LNP. The LNPs, combined with additional components, can be the same or separate LNPs. In other words, the same LNP can be PEG modified and sterol modified or, alternatively, a first LNP can be PEG modified and a second LNP can be sterol modified. Optionally, the first and second modified LNPs can be combined.

In certain embodiments, prior to encapsulating LNPs can have a size in a range from about 10 nm to 500 nm, or from about 50 nm to about 200 nm, or from 75 nm to about 125 nm.

In certain embodiments concerning LNP, the LNP is described by Billingsley et al., Nano Lett. 2020, 20, 1578 or International Patent Publication WO 2021/077066 (both of which are hereby incorporated by reference herein in their entirety). Billingsley et al., and WO 2021/077066 describe LNPs containing lipid-anchored PEG, cholesterol, phospholipid and ionizable lipids. In certain embodiments, the LNP contains a C14-4 polyamine core and/or has a particle size of about 70 nm. C14-4 has the following structure.

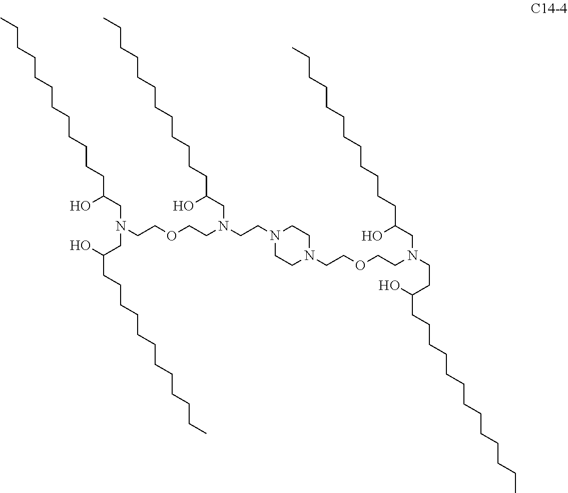 [SMILES: CCCCCCCCCCCCCC(O)CCN(CCOCCN1CCN(CCN(CCOCCN(CC(O)CCCCCCCCCCCC)CC(O)CCCCCCCCCCCC)CC(O)CCCCCCCCCCCC)CC1)CC(O)CCCCCCCCCCCC]

In certain embodiments concerning LNP, the LNP is made up of a cationic lipid or lipopeptide described by U.S. Pat. Nos. 10,493,031, 10,682,374 or WO2021/077066 (each of which is hereby incorporated by reference herein in its entirety). In certain embodiments, the LNP contains a cationic lipid, a cholesterol-based lipid, and/or one or more PEG-modified lipid. In certain embodiments the LNP contains cKK-E12 (Dong et al., PNAS 2014, 111(11), 3955):

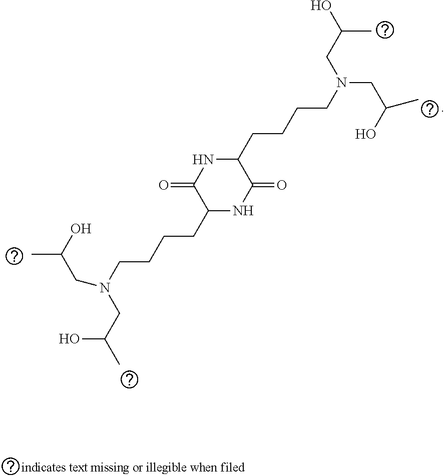 [SMILES: CC(O)CN(CCCCC1NC(=O)C(CCCCN(CC(C)O)CC(C)O)NC1=O)CC(C)O]

In certain embodiments concerning the LNP, the LNP comprises Lipid 1, 2, 3, 4, 5, 6, 7, 8, 9, or 10 as described by Sabnis et al., Molecular Therapy 2018, vol. 26, No. 6, 1509-1519 (hereby incorporated by reference herein in its entirety). In certain embodiments the LNP comprises Lipid 5, 8, 9, 10, or 11 described in Sabnis et al. Lipid 9 has the structure:

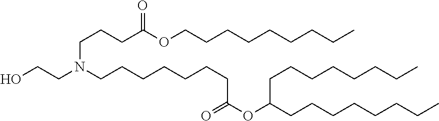 [SMILES: CCCCCCCCCOC(=O)CCCN(CCO)CCCCCCCC(=O)OC(CCCCCCCC)CCCCCCCC]

Additional lipids that may be utilized include those described by Roces et al., Pharmaceutics 2020, 12,1095; Jayaraman et al; Angew. Chem. Int. Ed. 2012, 51, 8529-8533; and Maier et al., www.moleculartherapy.org Vol.21, No. 8, 1570-1578, 2013; Liu et al., Adv. Mater. 2019, 31, 1902575, e.g., BAMEA-O16B); Cheng et al., Adv. Mater. 2018, 30, 1805308, e.g., 5A2-SC8; Hajj and Ball, Small 2019, 15, 1805097, e.g., 306Oi10 (each of which are hereby incorporated by reference herein in their entirety); and Du et al., Patent Application Publication No. US2016/0376224, e.g., compounds 1-46, as shown in Table 1 below).

In certain embodiments the LNP contains one or more lipids as provided in Table 1 or a pharmaceutically acceptable salt thereof.

TABLE 1

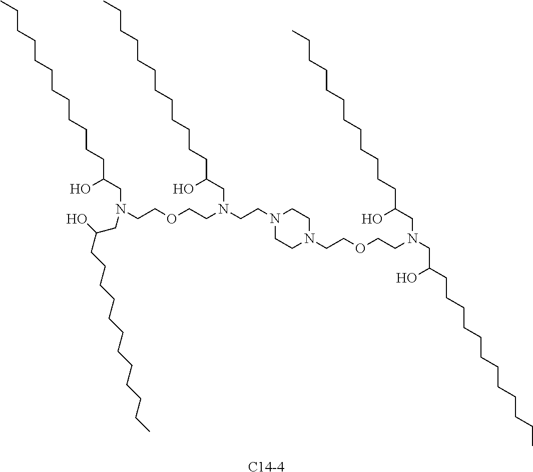 [SMILES: CCCCCCCCCCCCC(O)CN(CCOCCN(CC(O)CCCCCCCCCCCC)CC(O)CCCCCCCCCCCC)CCN1CCN(CCOCCN(CC(O)CCCCCCCCCCCC)CC(O)CCCCCCCCCCCC)CC1 ; "C14-4"]

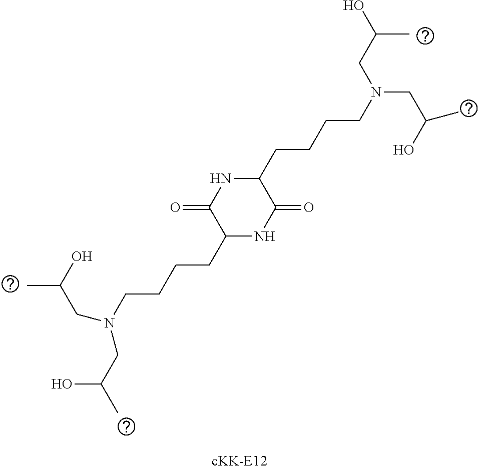 [SMILES: CC(O)CN(CCCCC1NC(=O)C(CCCCN(CC(C)O)CC(C)O)NC1=O)CC(C)O]

(6Z,9Z,28Z,31Z)-heptatriaconta-6,9,28,31-tetraen-19-yl 4-(dimethylamino)butanoate (DLin-M-C3-DMA, also known as MC3)

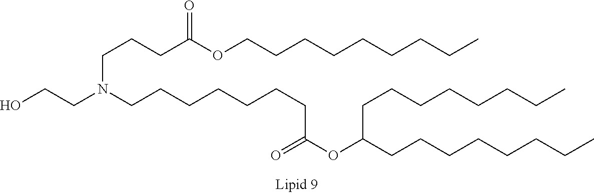 [SMILES: CCCCCCCCCOC(=O)CCCN(CCO)CCCCCCCC(=O)OC(CCCCCCCC)CCCCCCCC]

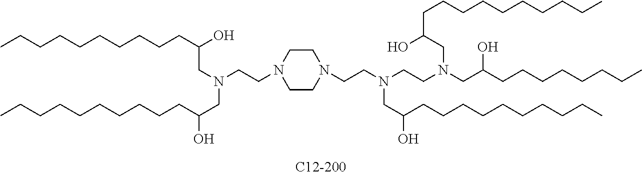 [SMILES: CCCCCCCCCCC(O)CN(CCN1CCN(CCN(CC(O)CCCCCCCCCC)CC(O)CCCCCCCCCC)CC1)CCN(CC(O)CCCCCCCC)CC(O)CCCCCCCCCC]

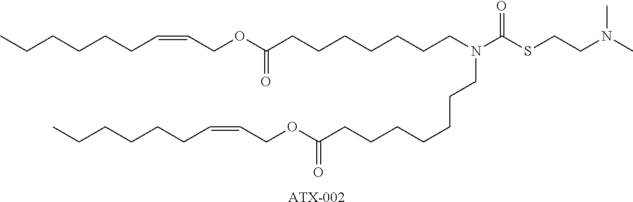

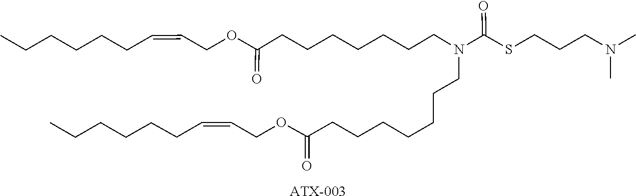

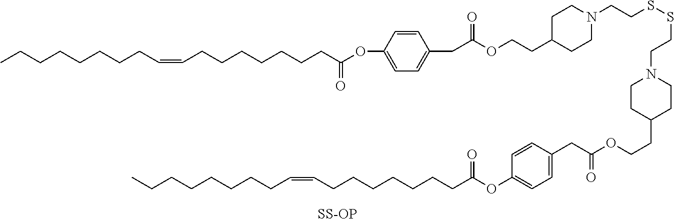

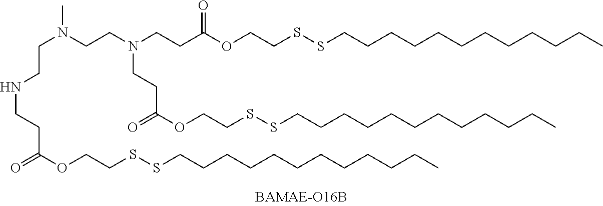 [SMILES: CCCCCCCCCCCCSSCCOC(=O)CCNCCN(C)CCN(CCC(=O)OCCSSCCCCCCCCCCCC)CCC(=O)OCCSSCCCCCCCCCCCC]

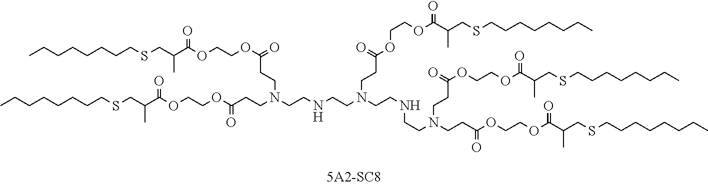 [SMILES: CCCCCCCCSCC(C)C(=O)OCCOC(=O)CCN(CCNCCN(CCC(=O)OCCOC(=O)C(C)CSCCCCCCCC)CCC(=O)OCCOC(=O)C(C)CSCCCCCCCC)CCNCCN(CCC(=O)OCCOC(=O)C(C)CSCCCCCCCC)CCC(=O)OCCOC(=O)C(C)CSCCCCCCCC]

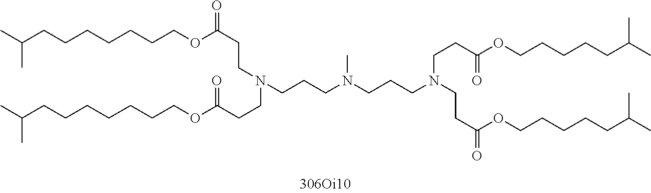 [SMILES: CC(C)CCCCCCCOC(=O)CCN(CCCN(C)CCCN(CCC(=O)OCCCCCC(C)C)CCC(=O)OCCCCCC(C)C)CCC(=O)OCCCCCCCC(C)C]

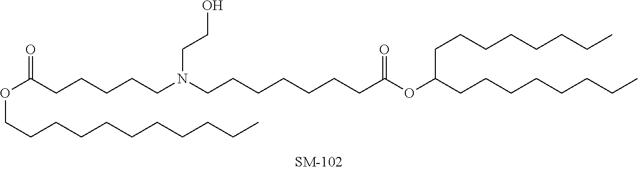 [SMILES: CCCCCCCCCCCOC(=O)CCCCCN(CCO)CCCCCCCC(=O)OC(CCCCCCCC)CCCCCCCC]

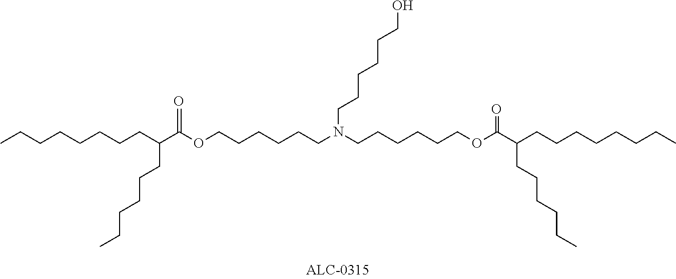 [SMILES: CCCCCCCCC(CCCCCC)C(=O)OCCCCCCN(CCCCCCO)CCCCCCOC(=O)C(CCCCCC)CCCCCCCC]

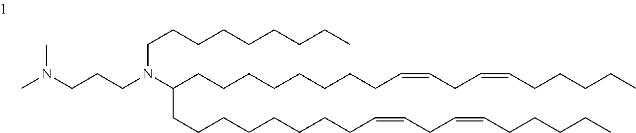 [SMILES: CCCCC/C=C\C/C=C\CCCCCCCCC(CCCCCCCC/C=C\C/C=C\CCCCC)N(CCCCCCCCC)CCCN(C)C]

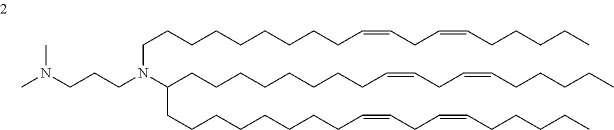 [SMILES: CCCCC/C=C\C/C=C\CCCCCCCCCN(CCCN(C)C)C(CCCCCCCC/C=C\C/C=C\CCCCC)CCCCCCCC/C=C\C/C=C\CCCCC]

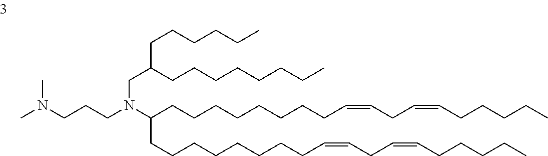 [SMILES: CCCCC/C=C\C/C=C\CCCCCCCCC(CCCCCCCC/C=C\C/C=C\CCCCC)N(CCCN(C)C)CC(CCCCCC)CCCCCCCC]

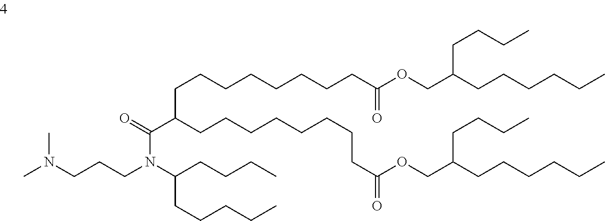 [SMILES: CCCCCCC(CCCC)COC(=O)CCCCCCCCC(CCCCCCCCC(=O)OCC(CCCC)CCCCCC)C(=O)N(CCCN(C)C)C(CCCC)CCCCC]

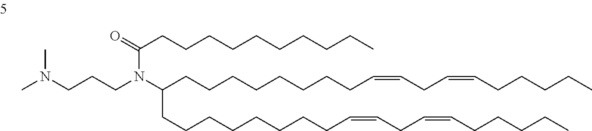 [SMILES: CCCCC/C=C\C/C=C\CCCCCCCCC(CCCCCCCC/C=C\C/C=C\CCCCC)N(CCCN(C)C)C(=O)CCCCCCCCCC]

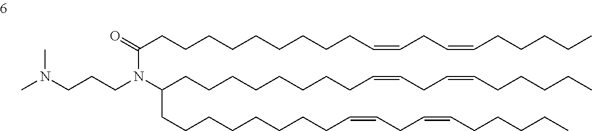 [SMILES: CCCCC/C=C\C/C=C\CCCCCCCCCC(=O)N(CCCN(C)C)C(CCCCCCCC/C=C\C/C=C\CCCCC)CCCCCCCC/C=C\C/C=C\CCCCC]

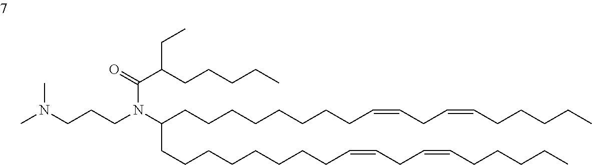 [SMILES: CCCCC/C=C\C/C=C\CCCCCCCCC(CCCCCCCC/C=C\C/C=C\CCCCC)N(CCCN(C)C)C(=O)C(CC)CCCCC]

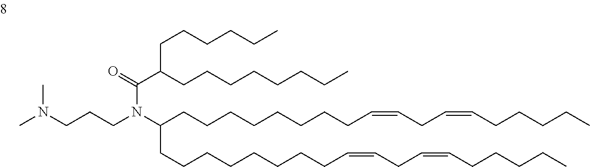 [SMILES: CCCCC/C=C\C/C=C\CCCCCCCCC(CCCCCCCC/C=C\C/C=C\CCCCC)N(CCCN(C)C)C(=O)C(CCCCCC)CCCCCCCC]

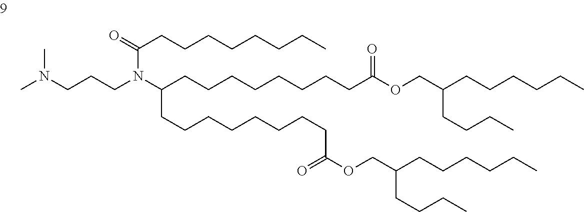 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

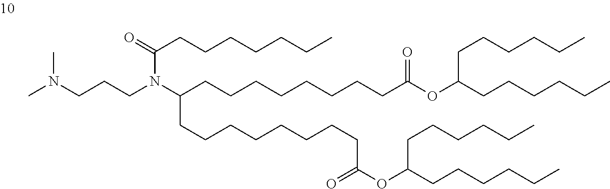 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

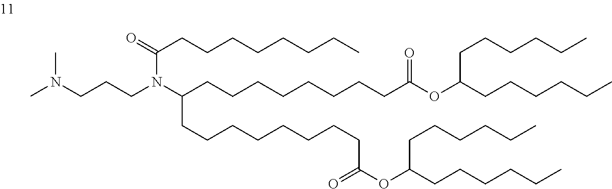 [SMILES: CCCCCCCCC(=O)N(CCCN(C)C)C(CCCCCCCCC(=O)OC(CCCCCC)CCCCCC)CCCCCCCCC(=O)OC(CCCCCC)CCCCCC]

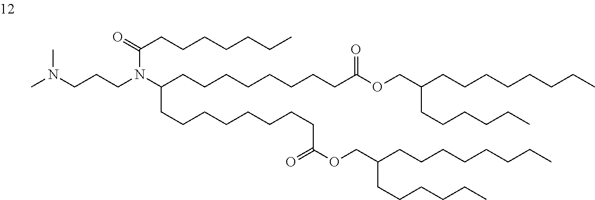 [SMILES: CCCCCCCCC(CCCCCC)COC(=O)CCCCCCCCC(CCCCCCCCC(=O)OCC(CCCCCC)CCCCCCCC)N(CCCN(C)C)C(=O)CCCCCCC]

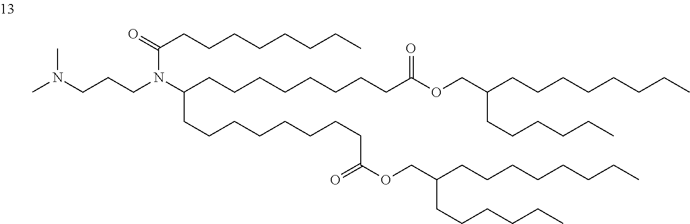 [SMILES: CCCCCCCCC(=O)N(CCCN(C)C)C(CCCCCCCCC(=O)OCC(CCCCCC)CCCCCCCC)CCCCCCCCC(=O)OCC(CCCCCC)CCCCCCCC]

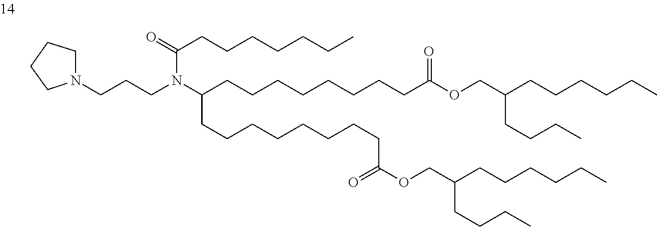 [SMILES: CCCCCCCC(=O)N(CCCN1CCCC1)C(CCCCCCCCC(=O)OCC(CCCC)CCCCCC)CCCCCCCCC(=O)OCC(CCCC)CCCCCC]

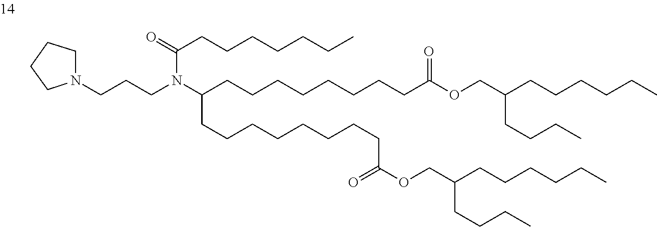 [SMILES: CCCCCCCC(=O)N(CCCN1CCCC1)C(CCCCCCCCC(=O)OCC(CCCC)CCCCCC)CCCCCCCCC(=O)OCC(CCCC)CCCCCC]

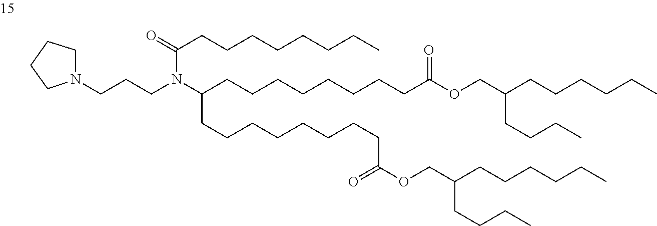 [SMILES: CCCCCCCCC(=O)N(CCCN1CCCC1)C(CCCCCCCCC(=O)OCC(CCCC)CCCCCC)CCCCCCCCC(=O)OCC(CCCC)CCCCCC]

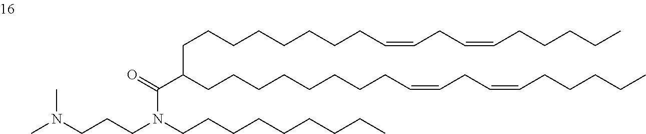 [SMILES: CCCCC/C=C\C/C=C\CCCCCCCCC(CCCCCCCC/C=C\C/C=C\CCCCC)C(=O)N(CCCCCCCCC)CCCN(C)C]

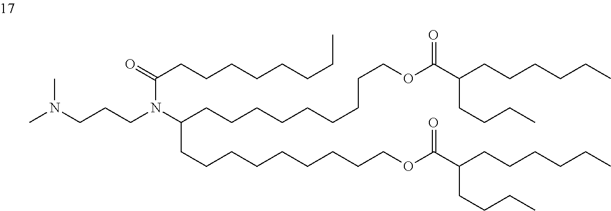 [SMILES: CCCCCCCCC(=O)N(CCCN(C)C)C(CCCCCCCCCOC(=O)C(CCCC)CCCCCC)CCCCCCCCCOC(=O)C(CCCC)CCCCCC]

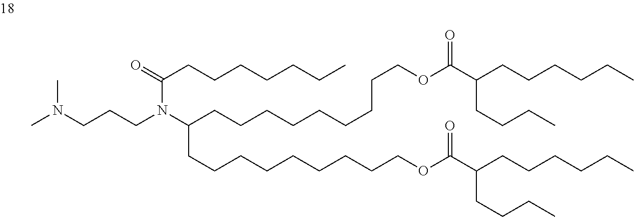 [SMILES: CCCCCCCC(=O)N(CCCN(C)C)C(CCCCCCCCCOC(=O)C(CCCC)CCCCCC)CCCCCCCCCOC(=O)C(CCCC)CCCCCC]

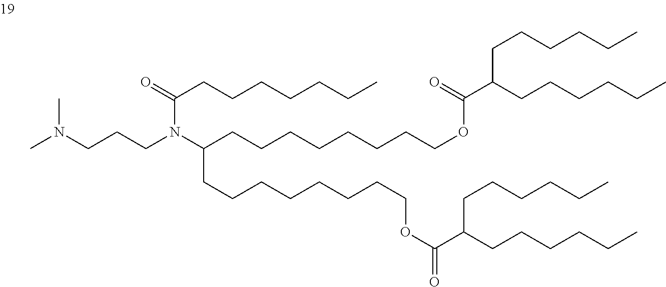 [SMILES: CCCCCCCC(=O)N(CCCN(C)C)C(CCCCCCCCOC(=O)C(CCCCCC)CCCCCC)CCCCCCCCOC(=O)C(CCCCCC)CCCCCC]

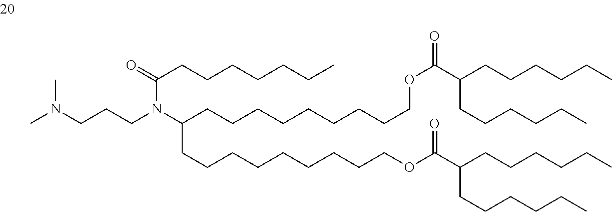 [SMILES: CCCCCCCC(=O)N(CCCN(C)C)C(CCCCCCCCCOC(=O)C(CCCCCC)CCCCCC)CCCCCCCCCOC(=O)C(CCCCCC)CCCCCC]

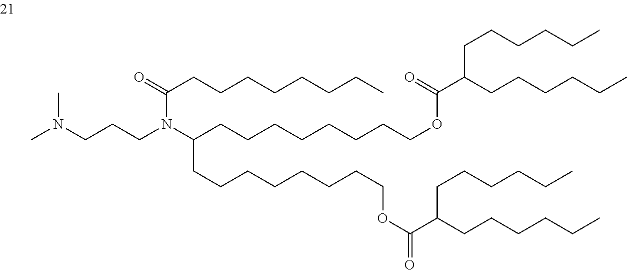 [SMILES: CCCCCCCCC(=O)N(CCCN(C)C)C(CCCCCCCCOC(=O)C(CCCCCC)CCCCCC)CCCCCCCCOC(=O)C(CCCCCC)CCCCCC]

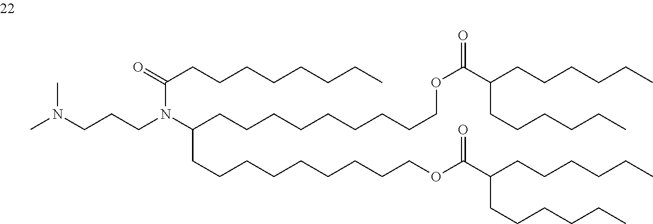 [SMILES: CCCCCCCCC(=O)N(CCCN(C)C)C(CCCCCCCCCOC(=O)C(CCCCCC)CCCCCC)CCCCCCCCCOC(=O)C(CCCCCC)CCCCCC]

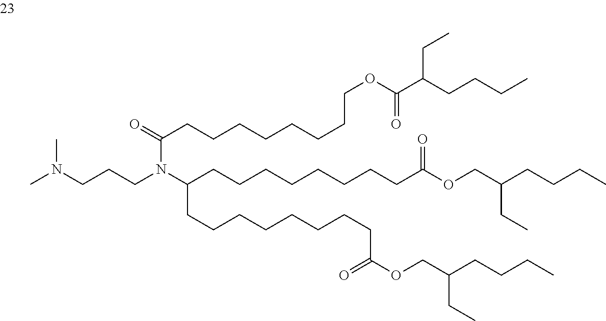 [SMILES: CCCCC(CC)COC(=O)CCCCCCCCC(CCCCCCCCC(=O)OCC(CC)CCCC)N(CCCN(C)C)C(=O)CCCCCCCCOC(=O)C(CC)CCCC]

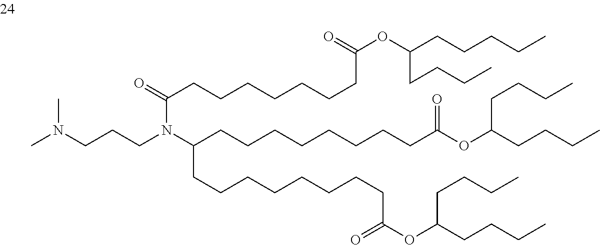 [SMILES: CCCCCC(CCCC)OC(=O)CCCCCCCC(=O)N(CCCN(C)C)C(CCCCCCCCC(=O)OC(CCCC)CCCC)CCCCCCCCC(=O)OC(CCCC)CCCC]

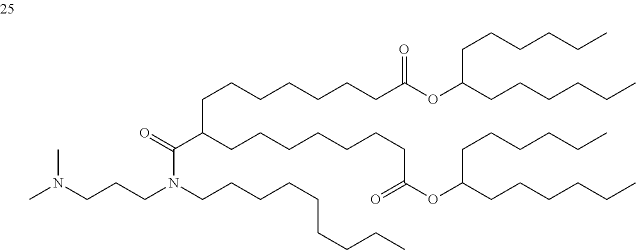 [SMILES: CCCCCCCCCN(CCCN(C)C)C(=O)C(CCCCCCCC(=O)OC(CCCCCC)CCCCCC)CCCCCCCC(=O)OC(CCCCCC)CCCCCC]

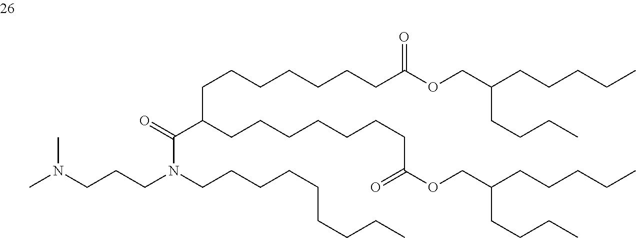 [SMILES: CCCCCCCCCN(CCCN(C)C)C(=O)C(CCCCCCCC(=O)OCC(CCCC)CCCCC)CCCCCCCC(=O)OCC(CCCC)CCCCC]

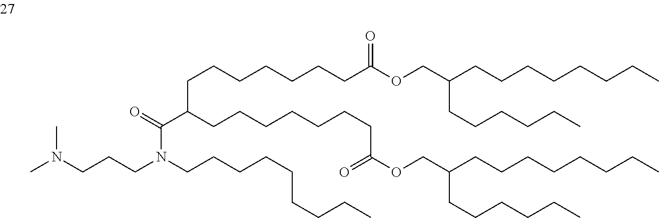 [SMILES: CCCCCCCCCN(CCCN(C)C)C(=O)C(CCCCCCCC(=O)OCC(CCCCCC)CCCCCCCC)CCCCCCCC(=O)OCC(CCCCCC)CCCCCCCC]

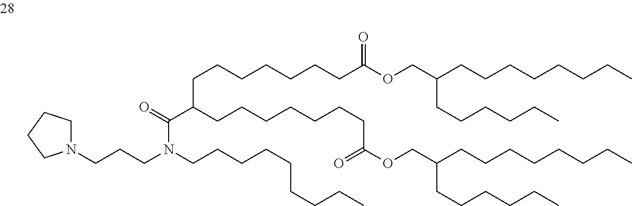 [SMILES: CCCCCCCCCN(CCCN1CCCC1)C(=O)C(CCCCCCCC(=O)OCC(CCCCCC)CCCCCCCC)CCCCCCCC(=O)OCC(CCCCCC)CCCCCCCC]

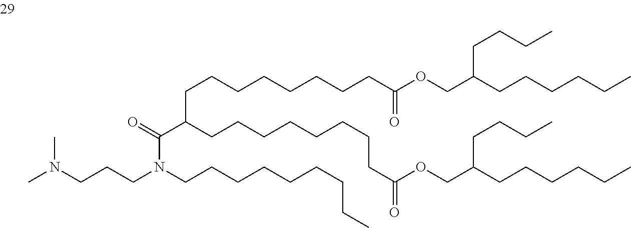 [SMILES: CCCCCCCCCN(CCCN(C)C)C(=O)C(CCCCCCCCC(=O)OCC(CCCC)CCCCCC)CCCCCCCCC(=O)OCC(CCCC)CCCCCC]

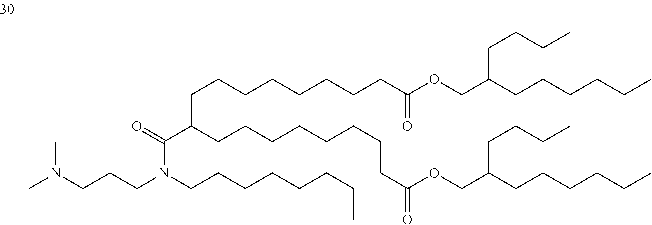 [SMILES: CCCCCCCCN(CCCN(C)C)C(=O)C(CCCCCCCCC(=O)OCC(CCCC)CCCCCC)CCCCCCCCC(=O)OCC(CCCC)CCCCCC]

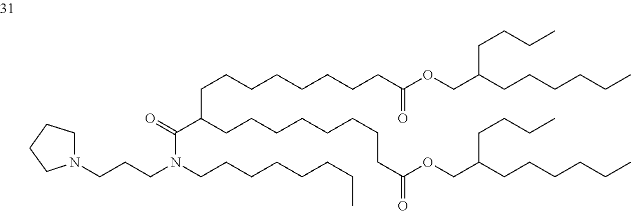 [SMILES: CCCCCCCCN(CCCN1CCCC1)C(=O)C(CCCCCCCCC(=O)OCC(CCCC)CCCCCC)CCCCCCCCC(=O)OCC(CCCC)CCCCCC]

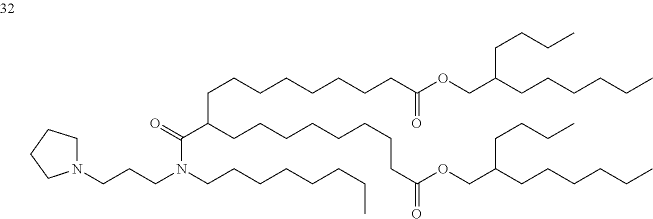 [SMILES: CCCCCCCCN(CCCN1CCCC1)C(=O)C(CCCCCCCCC(=O)OCC(CCCC)CCCCCC)CCCCCCCCC(=O)OCC(CCCC)CCCCCC]

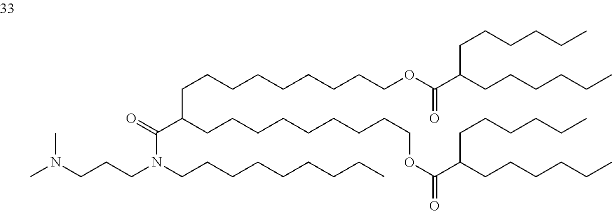 [SMILES: CCCCCCCCCN(CCCN(C)C)C(=O)C(CCCCCCCCCOC(=O)C(CCCCCC)CCCCCC)CCCCCCCCCOC(=O)C(CCCCCC)CCCCCC]

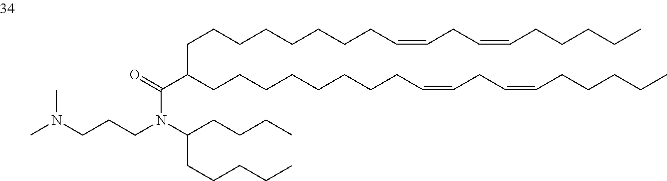 [SMILES: CCCCC/C=C\C/C=C\CCCCCCCCC(CCCCCCCC/C=C\C/C=C\CCCCC)C(=O)N(CCCN(C)C)C(CCCC)CCCCC]

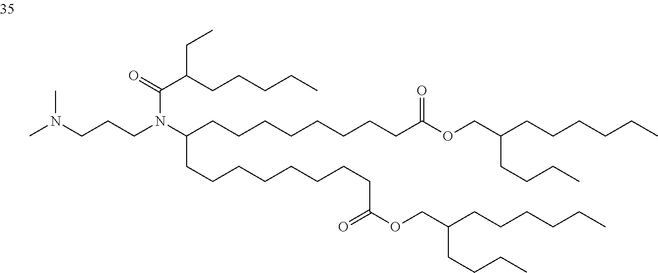 [SMILES: CCCCCCC(CCCC)COC(=O)CCCCCCCCC(CCCCCCCCC(=O)OCC(CCCC)CCCCCC)N(CCCN(C)C)C(=O)C(CC)CCCCC]

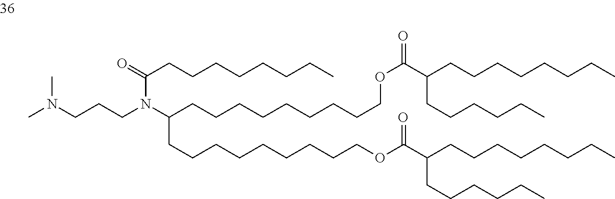 [SMILES: CCCCCCCCC(=O)N(CCCN(C)C)C(CCCCCCCCCOC(=O)C(CCCCCC)CCCCCCCC)CCCCCCCCCOC(=O)C(CCCCCC)CCCCCCCC]

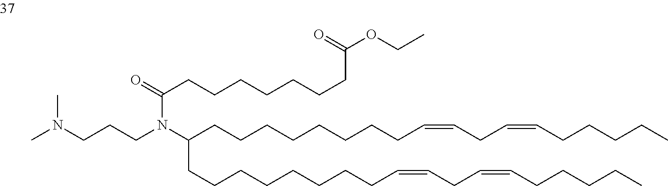 [SMILES: CCCCC/C=C\C/C=C\CCCCCCCCC(CCCCCCCC/C=C\C/C=C\CCCCC)N(CCCN(C)C)C(=O)CCCCCCCC(=O)OCC]

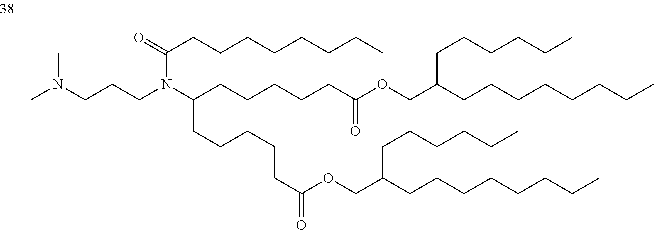 [SMILES: CCCCCCCCC(=O)N(CCCN(C)C)C(CCCCCC(=O)OCC(CCCCCC)CCCCCCCC)CCCCCC(=O)OCC(CCCCCC)CCCCCCCC]

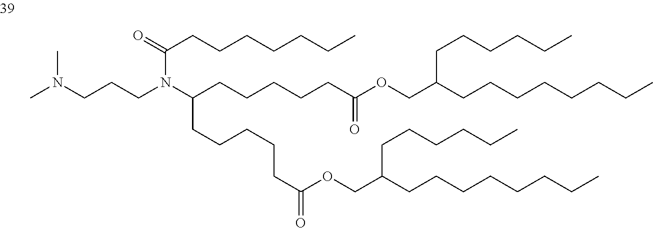 [SMILES: CCCCCCCCC(CCCCCC)COC(=O)CCCCCC(CCCCCC(=O)OCC(CCCCCC)CCCCCCCC)N(CCCN(C)C)C(=O)CCCCCCC]

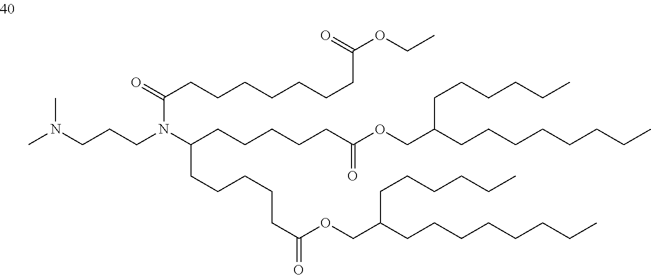 [SMILES: CCCCCCCCC(CCCCCC)COC(=O)CCCCCC(CCCCCC(=O)OCC(CCCCCC)CCCCCCCC)N(CCCN(C)C)C(=O)CCCCCCCC(=O)OCC]

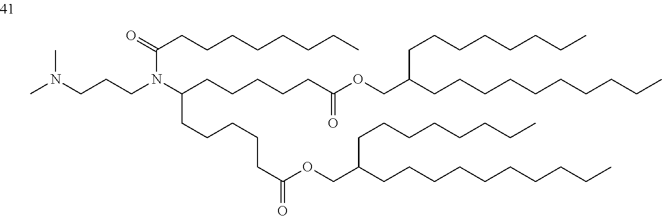 [SMILES: CCCCCCCCCCC(CCCCCCCC)COC(=O)CCCCCC(CCCCCC(=O)OCC(CCCCCCCC)CCCCCCCCCC)N(CCCN(C)C)C(=O)CCCCCCCC]

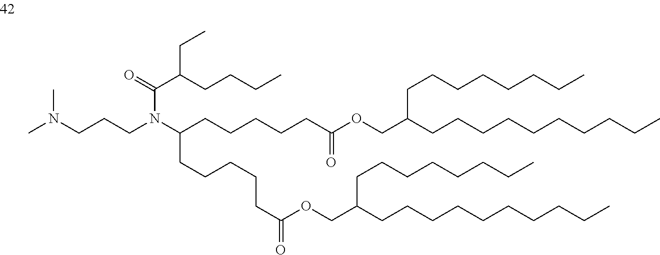 [SMILES: CCCCCCCCCCC(CCCCCCCC)COC(=O)CCCCCC(CCCCCC(=O)OCC(CCCCCCCC)CCCCCCCCCC)N(CCCN(C)C)C(=O)C(CC)CCCC]

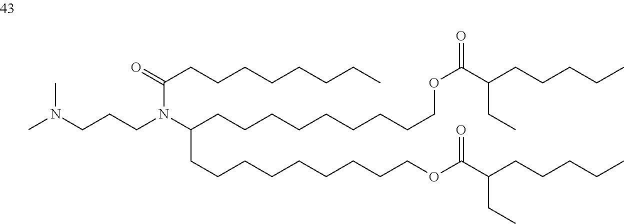 [SMILES: CCCCCCCCC(=O)N(CCCN(C)C)C(CCCCCCCCCOC(=O)C(CC)CCCCC)CCCCCCCCCOC(=O)C(CC)CCCCC]

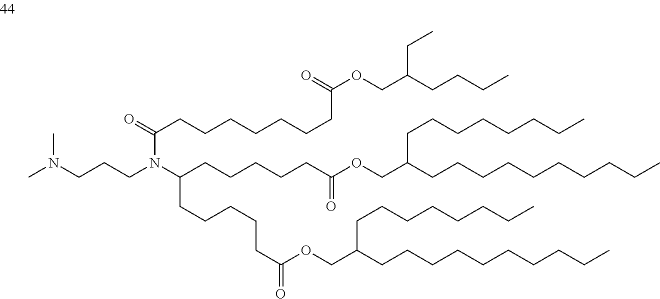 [SMILES: CCCCCCCCCCC(CCCCCCCC)COC(=O)CCCCCC(CCCCCC(=O)OCC(CCCCCCCC)CCCCCCCCCC)N(CCCN(C)C)C(=O)CCCCCCCC(=O)OCC(CC)CCCC]

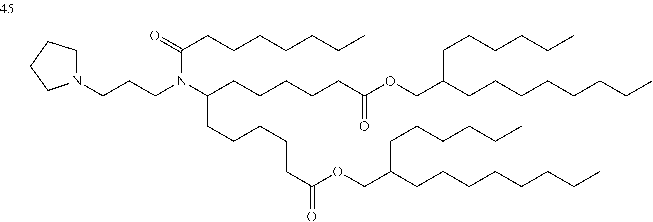 [SMILES: CCCCCCCCC(CCCCCC)COC(=O)CCCCCC(CCCCCC(=O)OCC(CCCCCC)CCCCCCCC)N(CCCN1CCCC1)C(=O)CCCCCCC]

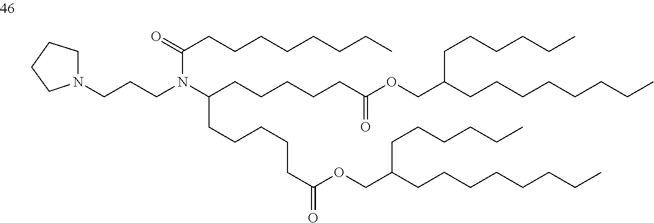 [SMILES: CCCCCCCCC(=O)N(CCCN1CCCC1)C(CCCCCC(=O)OCC(CCCCCC)CCCCCCCC)CCCCCC(=O)OCC(CCCCCC)CCCCCCCC]

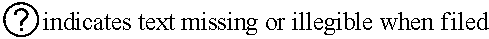

In certain embodiments, the LNP comprises mol % of the following components: one or more cationic lipids from about 20% to 65%, one or more phospholipid lipids from about 1% to about 50%, one or more PEG-conjugated lipid from about 0.1% to 10%, and cholesterol from about 0% to about 70%; one or more cationic lipids from about 20% to 50%, one or more phospholipid lipids from about 5% to about 20%, one or more PEG-conjugated lipid from about 0.1% to 5%, and cholesterol from about 20% to about 60%; in additional embodiments the phospholipid lipid is a neutral lipid; and the phospholipid lipid is DOPE or DSPC.

### Polymer-Based Systems

Polymer-based delivery systems are well known in the art, and any suitable polymer-based delivery system or polymeric nanoparticle known to those skilled in the art in view of the present disclosure can be used in the invention. DNA can be entrapped into the polymeric matrix of polymeric nanoparticles or can be adsorbed or conjugated on the surface of the nanoparticles. Examples of commonly used polymers for gene delivery include, e.g., poly(lactic-co-glycolic acid) (PLGA), poly lactic acid (PLA), poly(ethylene imine) (PEI), chitosan, dendrimers, polyanhydride, polycaprolactone, and polymethacrylates.

The polymeric-based non-viral vectors can have different sizes, ranging from about 1 nm to about 1000 nm, optionally from about 10 nm to about 500 nm, optionally from about 50 nm to about 200 nm, optionally about 100 nm to about 150 nm, optionally about 150 nm or less.

### Protein-Based Systems

Protein-based delivery systems are well known in the art, and any suitable protein-based delivery system or cell-penetrating peptide (CPP) known to those skilled in the art in view of the present disclosure can be used in the invention.

CPPs are short peptides (6-30 amino acid residues) that are potentially capable of intracellular penetration to deliver therapeutic molecules. The majority of CPPs consists mainly of arginine and lysine residues, making them cationic and hydrophilic, but CPPs can also be amphiphilic, anionic, or hydrophobic. CPPs can be derived from natural biomolecules (e.g., Tat, an HIV-1 protein), or obtained by synthetic methods (e.g., poly-L-lysine, polyarginine) (Singh et al., Drug Deliv. 2018;25(1):1996-2006). Examples of CPPs include, e.g., cationic CPPs (highly positively charged) (e.g., the Tat peptide, penetratin, protamine, poly-L-lysine, polyarginine, etc.); amphipathic CPPs (chimeric or fused peptides, constructed from different sources, contain both positively and negatively charged amino acid sequences) (e.g., transportan, VTS, bactenecin-7 (Bac7), proline-rich peptide (PPR), SAP (VRLPPP)₃, TP10, pep-1, MPG, etc.); membranotropic CPPs (exhibit both hydrophobic and amphipathic nature simultaneously, and comprise both large aromatic residues and small residues) (e.g., gH625, SPIONs-PEG-CPP NPs, etc.); and hydrophobic CPPs (contain only non-polar motifs or residues) (e.g., SG3, PFVYLI, pep-7, fibroblast growth factors (FGF), etc.).

The protein-based non-viral vectors can have different sizes, ranging from about 1 nm to about 1000 nm, optionally from about 10 nm to about 500 nm, optionally from about 50 nm to about 200 nm, optionally about 100 nm to about 150 nm, optionally about 150 nm or less.

### Peptide Cage Systems

Peptide cage-based delivery systems are well known in the art, and any suitable peptide cage-based delivery system known to those skilled in the art in view of the present disclosure can be used in the invention. In general, any proteinaceous material that is able to be assembled into a cage-like structure, forming a constrained internal environment, can be used. Several different types of protein “shells” can be assembled and loaded with different types of materials. For example, protein cages comprising a shell of viral coat protein(s) (e.g., from the Cowpea Chlorotic Mottle Virus (CCMV) protein coat) that encapsulate a non-viral material, as well as protein cages formed from non-viral proteins have been described (see, e.g., U.S. Pat. Nos. 6,180,389 and 6,984,386, U.S. Patent Application 20040028694, and U.S. Patent Application 20090035389, incorporated herein in their entity). Peptide cages can comprise a proteinaceous shell that self-assembles to form a protein cage (e.g., a structure with an interior cavity which is either naturally accessible to the solvent or can be made to be so by altering solvent concentration, pH, equilibria ratios).

Examples of protein cages derived from non-viral proteins include, e.g., ferritins and apoferritins, derived from both eukaryotic and prokaryotic species, e.g., 12 and 24 subunit ferritins; and protein cages formed from heat shock proteins (HSPs), e.g., the class of 24 subunit heat shock proteins that form an internal core space, the small HSP of Methanococcus jannaschii, the dodecameric Dsp HSP of E. coli, the MrgA protein, etc. As will be appreciated by those in the art, the monomers of the protein cages can be naturally occurring or variant forms, including amino acid substitutions, insertions, and deletions (e.g., fragments) that can be made.

The protein cages can have different core sizes, ranging from about 1 nm to about 1000 nm, optionally from about 10 nm to about 500 nm, optionally from about 50 nm to about 200 nm, optionally about 100 nm to about 150 nm, optionally about 150 nm or less.

### Therapeutic Nucleic Acids

The terms “nucleic acid” and “polynucleotide” are used interchangeably herein to refer to all forms of nucleic acid, oligonucleotides, including deoxyribonucleic acid (DNA) and ribonucleic acid (RNA). Nucleic acids include genomic DNA, cDNA, antisense DNA/RNA, plasmid DNA, linear DNA, (poly- and oligo-nucleotide), chromosomal DNA, spliced or unspliced mRNA, rRNA, tRNA inhibitory DNA or RNA (RNAi, e.g., small or short hairpin (sh)RNA, microRNA (miRNA), small or short interfering (si)RNA, trans-splicing RNA, or antisense RNA), locked nucleic acid analogue (LNA), oligonucleotide DNA (ODN) single and double stranded, immunostimulating sequence (ISS), riboswitches and ribozymes.

Nucleic acids include naturally occurring, synthetic, and intentionally modified or altered polynucleotides. Nucleic acids can be single, double, or triplex, linear or circular, and can be of any length. In discussing nucleic acids, a sequence or structure of a particular polynucleotide can be described herein according to the convention of providing the sequence in the 5′ to 3′ direction.

In certain embodiments, the nucleic acid agent is a single-stranded (ssDNA) or a double-stranded DNA (dsDNA) molecule. In certain embodiments, the nucleic acid agent is for therapeutic use, e.g. an ssDNA or dsDNA encoding a therapeutic transgene. In certain embodiments, the dsDNA molecule is a minicircle, a nanoplasmid, open linear duplex DNA or a closed-ended linear duplex DNA (CELiD/ceDNA/doggybone DNA). In certain embodiments, the ssDNA molecule is a closed circular or an open linear DNA.

According to the instant invention, ssDNA and dsDNA molecules comprising therapeutic transgenes can be produced by conventional techniques that are well known to those of skill in the art.

A “transgene” is used herein to conveniently refer to a nucleic acid that is intended or has been introduced into a cell or organism. Transgenes include any nucleic acid, such as a heterologous polynucleotide sequence or a heterologous nucleic acid encoding a protein or peptide or a nucleic acid (e.g., miRNA, etc.). The term transgene and heterologous nucleic acid/polynucleotide sequences are used interchangeably herein.

Typically, “therapeutic transgene” of the instant invention comprises an expression cassette. The term “expression cassette”, as used herein, refers to a nucleic acid construct comprising nucleic acid elements sufficient for the expression of the nucleic acid molecule of the instant invention. Typically, an expression cassette comprises the nucleic acid molecule of the instant invention operatively linked to a promoter sequence. The term “operatively linked” refers to the association of two or more nucleic acid fragments on a single nucleic acid fragment so that the function of one is affected by the other. For example, a promoter is operatively linked with a coding sequence when it is capable of affecting the expression of that coding sequence (e.g., the coding sequence is under the transcriptional control of the promoter). Encoding sequences can be operatively linked to regulatory sequences in sense or antisense orientation. In certain embodiments, the promoter is a heterologous promoter. The term “heterologous promoter”, as used herein, refers to a promoter that is not found to be operatively linked to a given encoding sequence in nature.

As used herein, the term “operably linked” means that regulatory sequences having an effect on the expression of a transgene are placed in the appropriate positions relative to the sequence so as to affect expression of the transgene. This same definition is sometimes applied to the arrangement of transgenes and transcription control elements (e.g., promoters, enhancers, promoter/enhancers, and termination elements) in an expression vector. Transgenes can be operably linked to regulatory sequences in sense or antisense orientation. A wide variety of regulatory sequences that can be operably linked with transgenes are known in the art and can be used in the methods of the present invention. In certain embodiments, the operably linked regulatory sequence is a promoter, such as a liver specific promoter, or a promoter/enhancer, such as ApoE/hAAT.

In certain embodiments, an expression cassette can comprise additional elements, for example, an intron, an enhancer, a polyadenylation site, a woodchuck response element (WRE), and/or other elements known to affect expression levels of the encoding sequence. As used herein, the term “promoter” refers to a nucleotide sequence capable of controlling the expression of a coding sequence or functional RNA. In general, the nucleic acid molecule of the instant invention is located 3′ of a promoter sequence. In certain embodiments, a promoter sequence consists of proximal and more distal upstream elements and can comprise an enhancer element. An “enhancer” is a nucleotide sequence that can stimulate promoter activity. Enhancer elements are typically located upstream of a promoter element but can also be located downstream or within a promoter and can, for example, be an innate element of the promoter or a heterologous element inserted to enhance the level or tissue-specificity of a promoter. In certain embodiments, an expression cassette comprises a tissue-specific enhancer. In certain embodiments, the promoter is derived in its entirety from a native gene. In certain embodiments, the promoter is composed of different elements derived from different naturally occurring promoters. In certain embodiments, the promoter comprises a synthetic nucleotide sequence. It will be understood by those skilled in the art that different promoters will direct the expression of a gene in different tissues or cell types, or at different stages of development, or in response to different environmental conditions or to the presence or the absence of a drug or transcriptional co-factor. Ubiquitous, cell-type-specific, tissue-specific, developmental stage-specific, and conditional promoters, for example, drug-responsive promoters (e.g., tetracycline-responsive promoters) are well known to those of skill in the art. Examples of promoters include, but are not limited to, the phosphoglycerate kinase (PKG) promoter, CAG (composite of the CMV enhancer the chicken beta actin promoter (CBA) and the rabbit beta globin intron), NSE (neuronal specific enolase), synapsin or NeuN promoters, the SV40 early promoter, mouse mammary tumor virus LTR promoter, adenovirus major late promoter (Ad MLP), a herpes simplex virus (HSV) promoter, a cytomegalovirus (CMV) promoter such as the CMV immediate early promoter region (CMVIE), SFFV promoter, rous sarcoma virus (RSV) promoter, synthetic promoters, hybrid promoters, and the like. Other promoters can be of human origin or from other species, including from mice. Common promoters include, e.g., the human cytomegalovirus (CMV) immediate early gene promoter, the SV40 early promoter, the Rous sarcoma virus long terminal repeat, beta-actin promoter, rat insulin promoter, the phosphoglycerate kinase promoter, the human alpha-1 antitrypsin (hAAT) promoter, the transthyretin promoter, the thyroxine binding globulin (TBG) promoter and other liver-specific promoters, the desmin promoter and similar muscle-specific promoters, the EF1-alpha promoter, the CAG promoter and other constitutive promoters, hybrid promoters with multi-tissue specificity, promoters specific for neurons like synapsin and glyceraldehyde-3-phosphate dehydrogenase promoter, all of which are promoters well known and readily available to those of skill in the art, can be used to obtain high-level expression of the coding sequence of interest. In addition, sequences derived from non-viral genes, such as the murine metallothionein gene, will also find use herein. Such promoter sequences are commercially available from, e.g., Stratagene (San Diego, CA).

Operably linking the transgene to tissue specific regulatory elements provides for at least partial tissue tropism for transgene expression. In certain embodiments, the transgene is operably linked to a liver specific promoter and/or liver specific enhancer. Reference to “liver specific” indicates at least partial tissue tropism for transgene expression in the liver. Reference to “promoter/enhancer” indicates operably linked to an enhancer and promoter. Examples of promoters active in the liver include, without limitation, the transthyretin (TTR) gene promoter and mutant versions thereof (Anguela and Shen, U.S. Pat. No. 11,168,124, Anguela and Shen, International Publication No. WO2017/075619; Costa et al., 1991, Nucleic Acids Research, Vol. 19, No. 15 4139-4145); human alpha 1-antitrypsin (hAAT) promoter (Hafenrichter et al., Blood. 1994, Vol 84, No. 10. 3394-3404); the apolipoprotein A-I promoter; albumin (Miyatake et al., J. Virol., 1997, 71:5124-32); hepatitis B virus core promoter (Sandig et al., Gene Ther. 1996, 3: 1002-9); alpha-fetoprotein (AFP) (Arbuthnot et al., Hum. Gene. Ther., 1996, 7:1503-14); human Factor IX promoter; and thyroxin binding globulin (TBG) promoter. An example of an enhancer active in liver is apolipoprotein E (apoE) HCR-1 and HCR-2 (Allan et al., J. Biol. Chem., 1997, 272:29113-19). In certain embodiments the promoter/enhancer is ApoE/hAAT (Okuyama et al., 1996, Human Gene Therapy 7:637-645, and Miao et al., 2000, Molecular Therapy Vol. 1, No. 6). (Each of the references in the present paragraph are hereby incorporated by reference herein.)

In certain embodiments, the expression cassette comprises an appropriate secretory signal sequence that will allow the secretion of the polypeptide encoded by the nucleic acid molecule of the instant invention. As used herein, the term “secretory signal sequence” or variations thereof are intended to refer to amino acid sequences that function to enhance (as defined above) secretion of an operably linked polypeptide from the cell as compared with the level of secretion seen with the native polypeptide. By “enhanced” secretion, it is meant that the relative proportion of the polypeptide synthesized by the cell that is secreted from the cell is increased; it is not necessary that the absolute amount of secreted protein is also increased. In certain embodiments, essentially all (i.e., at least 95%, 97%, 98%, 99% or more) of the polypeptide is secreted. It is not necessary, however, that essentially all or even most of the polypeptide is secreted, as long as the level of secretion is enhanced as compared with the native polypeptide. Generally, secretory signal sequences are cleaved within the endoplasmic reticulum and, in certain embodiments, the secretory signal sequence is cleaved prior to secretion. It is not necessary, however, that the secretory signal sequence is cleaved as long as secretion of the polypeptide from the cell is enhanced and the polypeptide is functional. Thus, in certain embodiments, the secretory signal sequence is partially or entirely retained. The secretory signal sequence can be derived in whole or in part from the secretory signal of a secreted polypeptide (i.e., from the precursor) and/or can be in whole or in part synthetic. The length of the secretory signal sequence is not critical; generally, known secretory signal sequences are from about 10-15 to 50-60 amino acids in length. Further, known secretory signals from secreted polypeptides can be altered or modified (e.g., by substitution, deletion, truncation or insertion of amino acids) as long as the resulting secretory signal sequence functions to enhance secretion of an operably linked polypeptide. The secretory signal sequences of the instant invention can comprise, consist essentially of or consist of a naturally occurring secretory signal sequence or a modification thereof (as described above). Numerous secreted proteins and sequences that direct secretion from the cell are known in the art. The secretory signal sequence of the instant invention can further be in whole or in part synthetic or artificial. Synthetic or artificial secretory signal peptides are known in the art, see, e.g., Barash et al., Biochem. Biophys. Res. Comm. 294:835-42 (2002).

In certain embodiments, the instant invention includes a method of treating a disease by gene therapy using a therapeutic transgene.

In certain embodiments, a heterologous polynucleotide encodes GAA (acid alpha-glucosidase) for treatment of Pompe disease or another glycogen storage disease; ATP7B (copper transporting ATPase2) for treatment of Wilson's disease; alpha galactosidase A for treatment of Fabry's disease; ASS1 (arginosuccinate synthase) for treatment of Citrullinemia Type 1; beta-glucocerebrosidase for treatment of Gaucher disease Type 1; beta-hexosaminidase A for treatment of Tay Sachs disease; SERPING1 (C1 protease inhibitor or C1 esterase inhibitor) for treatment of hereditary angioedema (HAE), also known as C1 inhibitor deficiency type I and type II); or glucose-6-phosphatase for treatment of glycogen storage disease type I (GSDI).

In certain embodiments, a heterologous polynucleotide encodes insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), transforming growth factor α (TGFα), platelet-derived growth factor (PDGF), insulin growth factors I or II (IGF-I or IGF-II), TGFβ, activins, inhibins, bone morphogenic protein (BMP), nerve growth factor (NGF), brain-derived neurotrophic factor (BDNF), neurotrophins NT-3 or NT4/5, ciliary neurotrophic factor (CNTF), glial cell line derived neurotrophic factor (GDNF), neurturin, agrin, netrin-1 or netrin-2, hepatocyte growth factor (HGF), ephrins, noggin, sonic hedgehog or tyrosine hydroxylase.

In certain embodiments, a heterologous polynucleotide thrombopoietin (TPO), an interleukin (IL-1 through IL-36, etc.), monocyte chemoattractant protein, leukemia inhibitory factor, granulocyte-macrophage colony stimulating factor, Fas ligand, tumor necrosis factors α or β, interferons α, β, or γ, stem cell factor, flk-2/flt3 ligand, IgG, IgM, IgA, IgD or IgE, chimeric immunoglobulins, humanized antibodies, single chain antibodies, T cell receptors, chimeric T cell receptors, single chain T cell receptors, class I or class II MHC molecules.

In certain embodiments, a heterologous polynucleotide encodes CFTR (cystic fibrosis transmembrane regulator protein), a blood coagulation (clotting) factor (Factor XIII, Factor IX (FIX), Factor VIII (FVIII), Factor X, Factor VII, Factor VIIa, protein C, etc.) a gain of function blood coagulation factor, an antibody, retinal pigment epithelium-specific 65 kDa protein (RPE65), erythropoietin, LDL receptor, lipoprotein lipase, ornithine transcarbamylase, β-globin, α-globin, spectrin, α-antitrypsin, adenosine deaminase (ADA), a metal transporter (ATP7A or ATP7), sulfamidase, an enzyme involved in lysosomal storage disease (ARSA), hypoxanthine guanine phosphoribosyl transferase, β-25 glucocerebrosidase, sphingomyelinase, lysosomal hexosaminidase, branched-chain keto acid dehydrogenase, a hormone, a growth factor, insulin-like growth factor 1 or 2, platelet derived growth factor, epidermal growth factor, nerve growth factor, neurotrophic factor −3 and −4, brain-derived neurotrophic factor, glial derived growth factor, transforming growth factor α and β, a cytokine, α-interferon, β-interferon, interferon-γ, interleukin-2, interleukin-4, interleukin 12, granulocyte-macrophage colony stimulating factor, lymphotoxin, a suicide gene product, herpes simplex virus thymidine kinase, cytosine deaminase, diphtheria toxin, cytochrome P450, deoxycytidine kinase, tumor necrosis factor, a drug resistance protein, a tumor suppressor protein (e.g., p53, Rb, Wt-1, NF1, Von Hippel-Lindau (VHL), adenomatous polyposis coli (APC)), a peptide with immunomodulatory properties, a tolerogenic or immunogenic peptide or protein Tregitope or hCDR1, insulin, glucokinase, guanylate cyclase 2D (LCA-GUCY2D), Rab escort protein 1 (Choroideremia), LCA 5 (LCA-Lebercilin), ornithine ketoacid aminotransferase (Gyrate Atrophy), Retinoschisin 1 (X-linked Retinoschisis), USH1C (Usher's Syndrome 1C), X-linked retinitis pigmentosa GTPase (XLRP), MERTK (AR forms of RP: retinitis pigmentosa), DFNB1 (Connexin 26 deafness), ACHM 2, 3 and 4 (Achromatopsia), PKD-1 or PKD-2 (Polycystic kidney disease), TPP1, CLN2, a sulfatase, N-acetylglucosamine-1-phosphate transferase, cathepsin A, GM2-AP, NPC1, VPC2, a sphingolipid activator protein, one or more zinc finger nucleases for genome editing, or one or more donor sequences used as repair templates for genome editing.

In certain embodiments, a heterologous polynucleotide encodes erythropoietin (EPO) for treatment of anemia; interferon-alpha, interferon-beta, and interferon-gamma for treatment of various immune disorders, viral infections and cancer; an interleukin (IL), including any one of IL-1 through IL-36, and corresponding receptors, for treatment of various inflammatory diseases or immuno-deficiencies; a chemokine, including chemokine (C-X-C motif) ligand 5 (CXCL5) for treatment of immune disorders; granulocyte-colony stimulating factor (G-CSF) for treatment of immune disorders such as Crohn's disease; granulocyte-macrophage colony stimulating factor (GM-CSF) for treatment of various human inflammatory diseases; macrophage colony stimulating factor (M-CSF) for treatment of various human inflammatory diseases; keratinocyte growth factor (KGF) for treatment of epithelial tissue damage; chemokines such as monocyte chemoattractant protein-1 (MCP-1) for treatment of recurrent miscarriage, HIV-related complications, and insulin resistance; tumor necrosis factor (TNF) and receptors for treatment of various immune disorders; alphal-antitrypsin for treatment of emphysema or chronic obstructive pulmonary disease (COPD); alpha-L-iduronidase for treatment of mucopolysaccharidosis I (MPS I); ornithine transcarbamoylase (OTC) for treatment of OTC deficiency; phenylalanine hydroxylase (PAH) or phenylalanine ammonia-lyase (PAL) for treatment of phenylketonuria (PKU); lipoprotein lipase for treatment of lipoprotein lipase deficiency; apolipoproteins for treatment of apolipoprotein (Apo) A-I deficiency; low-density lipoprotein receptor (LDL-R) for treatment of familial hypercholesterolemia (FH); albumin for treatment of hypoalbuminemia; lecithin cholesterol acyltransferase (LCAT); carbamoyl synthetase I; argininosuccinate synthetase; argininosuccinate lyase; arginase; fumarylacetoacetate hydrolase; porphobilinogen deaminase; cystathionine beta-synthase for treatment of homocystinuria; branched chain ketoacid decarboxylase; isovaleryl-CoA dehydrogenase; propionyl CoA carboxylase; methylmalonyl-CoA mutase; glutaryl CoA dehydrogenase; insulin; pyruvate carboxylase; hepatic phosphorylase; phosphorylase kinase; glycine decarboxylase; H-protein; T-protein; cystic fibrosis transmembrane regulator (CFTR); ATP-binding cassette, sub-family A (ABC1), member 4 (ABCA4) for the treatment of Stargardt disease; or dystrophin.

In certain embodiments, the protein encoded by the heterologous polynucleotide comprises a gene editing nuclease. In certain embodiments, the gene editing nuclease comprises a zinc finger nuclease (ZFN) or a transcription activator-like effector nuclease (TALEN). In certain embodiments, the gene editing nuclease comprises a functional Type II CRISPR-Cas9.

The terms “polypeptides,” “proteins” and “peptides” are used interchangeably herein. The “polypeptides,” “proteins” and “peptides” encoded by the “polynucleotide sequences,” include full-length native sequences, as with naturally occurring proteins, as well as functional subsequences, modified forms or sequence variants so long as the subsequence, modified form or variant retains some degree of functionality of the native full-length protein. In the instant invention, such polypeptides, proteins, and peptides encoded by the polynucleotide sequences can be, but are not required to be, identical to the endogenous protein that is defective, or whose expression is insufficient, or deficient in the treated mammal.

In certain embodiments, the heterologous polynucleotide encodes an inhibitory nucleic acid selected from the group consisting of a siRNA, an antisense molecule, miRNA, RNAi, a ribozyme and an shRNA.

In certain embodiments, an inhibitory nucleic acid binds to a gene, a transcript of a gene, or a transcript of a gene associated with a polynucleotide repeat disease selected from the group consisting of a huntingtin (HTT) gene, a gene associated with dentatorubropallidoluysian atrophy (atrophin 1, ATN1), androgen receptor on the X chromosome in spinobulbar muscular atrophy, human Ataxin-1, -2, -3, and -7, Cav2.1 P/Q voltage-dependent calcium channel (CACNA1A), TATA-binding protein, Ataxin 8 opposite strand (ATXN8OS), Serine/threonine-protein phosphatase 2A 55 kDa regulatory subunit B beta isoform in spinocerebellar ataxia (type 1, 2, 3, 6, 7, 8, 12 17), FMR1 (fragile X mental retardation 1) in fragile X syndrome, FMR1 (fragile X mental retardation 1) in fragile X-associated tremor/ataxia syndrome, FMR1 (fragile X mental retardation 2) or AF4/FMR2 family member 2 in fragile XE mental retardation; Myotonin-protein kinase (MT-PK) in myotonic dystrophy; Frataxin in Friedreich's ataxia; a mutant of superoxide dismutase 1 (SOD1) gene in amyotrophic lateral sclerosis; a gene involved in pathogenesis of Parkinson's disease and/or Alzheimer's disease; apolipoprotein B (APOB) and proprotein convertase subtilisin/kexin type 9 (PCSK9), hypercholesterolemia; HIV Tat, human immunodeficiency virus transactivator of transcription gene, in HIV infection; HIV TAR, HIV TAR, human immunodeficiency virus transactivator response element gene, in HIV infection; C-C chemokine receptor (CCR5) in HIV infection; Rous sarcoma virus (RSV) nucleocapsid protein in RSV infection, liver-specific microRNA (miR-122) in hepatitis C virus infection; p53, acute kidney injury or delayed graft function kidney transplant or kidney injury acute renal failure; protein kinase N3 (PKN3) in advance recurrent or metastatic solid malignancies; LMP2, LMP2 also known as proteasome subunit beta-type 9 (PSMB 9), metastatic melanoma; LMP7, also known as proteasome subunit beta-type 8 (PSMB 8), metastatic melanoma; MECL1 also known as proteasome subunit beta-type 10 (PSMB 10), metastatic melanoma; vascular endothelial growth factor (VEGF) in solid tumors; kinesin spindle protein in solid tumors, apoptosis suppressor B-cell CLL/lymphoma (BCL-2) in chronic myeloid leukemia; ribonucleotide reductase M2 (RRM2) in solid tumors; Furin in solid tumors; polo-like kinase 1 (PLK1) in liver tumors, diacylglycerol acyltransferase 1 (DGAT1) in hepatitis C infection, beta-catenin in familial adenomatous polyposis; beta2 adrenergic receptor, glaucoma; RTP801/Redd1 also known as DAN damage-inducible transcript 4 protein, in diabetic macular edema (DME) or age-related macular degeneration; vascular endothelial growth factor receptor I (VEGFR1) in age-related macular degeneration or choroidal neovascularization, caspase 2 in non-arteritic ischaemic optic neuropathy; Keratin 6A N17K mutant protein in pachyonychia congenital; influenza A virus genome/gene sequences in influenza infection; severe acute respiratory syndrome (SARS) coronavirus genome/gene sequences in SARS infection; respiratory syncytial virus genome/gene sequences in respiratory syncytial virus infection; Ebola filovirus genome/gene sequence in Ebola infection; hepatitis B and C virus genome/gene sequences in hepatitis B and C infection; herpes simplex virus (HSV) genome/gene sequences in HSV infection, coxsackievirus B3 genome/gene sequences in coxsackievirus B3 infection; silencing of a pathogenic allele of a gene (allele-specific silencing) like torsin A (TOR1A) in primary dystonia, pan-class I and HLA-allele specific in transplant; and mutant rhodopsin gene (RHO) in autosomal dominantly inherited retinitis pigmentosa (adRP).

### Immune Cell Modulators

In certain embodiments, the instant invention relates to a method of producing a therapeutically effective non-viral gene therapy in a subject comprising administration to the subject at least one immune cell modulator, e.g., at least one phagocyte-depleting agent, or at least one immunosuppressant.

As used herein, the term “phagocyte-depleting agent” refers to any agent that depletes or destroys phagocytes in a subject and/or interferes with one or more phagocyte functions. The phagocyte-depleting agents can target any phagocytes. Phagocytes, also referred to herein as phagocytic cells, phagocytic immune cells, phagocyte cells, or phagocyte immune cells, include, e.g., macrophages, monocytes, neutrophils, and dendritic cells. Langerhans cells are dendritic cells found in the skin. Mast cells, found in many tissues, including, e.g., lung or skin, can also act as phagocytes.

As used herein, the term “monocyte and/or macrophage-depleting agent” refers to any agent that depletes or destroys monocytes and/or macrophages in a subject and/or interferes with one or more monocyte and/or macrophage functions. The monocyte and/or macrophage-depleting agents can target any monocytes and/or macrophages. Macrophages are mononuclear phagocytes that are differentiated monocytes. In different tissues, macrophages are referred to by different names. Examples of tissue-specific, or resident, macrophages include, e.g., Kupffer cells in the liver, intestinal macrophages in the gut, microglial cells in the brain, alveolar macrophages in the lung, resident kidney macrophages, skin macrophages, red pulp macrophages in the spleen, and osteoclasts in bone. Examples of monocyte- and/or macrophage-depleting agents include, e.g., agents that target phagocytic immune cell markers, e.g., CD115 inhibiting agents, including but not limited to anti-CD115 antibodies or CD115 small molecule inhibitors; F4/80 inhibiting agents, including but not limited to anti-F4/80 antibodies or F4/80 small molecule inhibitors; CD68 inhibiting agents, including but not limited to anti-CD68 antibodies or CD68 small molecule inhibitors; CD11b inhibiting agents, including but not limited to anti-CD11b antibodies or CD11b small molecule inhibitors; the chemotherapeutic agent Trabectedin; intralipids; empty liposomes; and bisphosphonates, including but not limited to clodronate. In certain embodiments, the monocyte and/or macrophage-depleting agent is not clodronate. In certain embodiments. clodronate and at least one additional monocyte and/or macrophage-depleting agent are used together in a method of the invention.

As used herein, the term “neutrophil-depleting agent” refers to any agent that depletes or destroys neutrophils in a subject and/or interferes with one or more neutrophil functions. The neutrophil-depleting agents can target any neutrophils. Examples of neutrophil-depleting agents include, e.g., agents that target phagocytic immune cell markers, e.g., Ly6G inhibiting agents, including but not limited to anti-Ly6G antibodies or Ly6G small molecule inhibitors; CD177 inhibiting agents, including but not limited to anti-CD177 antibodies or CD177 small molecule inhibitors; CD14 inhibiting agents, including but not limited to anti-CD14 antibodies or CD14 small molecule inhibitors; CD15 inhibiting agents, including but not limited to anti-CD15 antibodies or CD15 small molecule inhibitors; CD11b inhibiting agents, including but not limited to anti-CD11b antibodies or CD11b small molecule inhibitors; CD16 inhibiting agents, including but not limited to anti-CD16 antibodies or CD16 small molecule inhibitors; CD32 inhibiting agents, including but not limited to anti-CD32 antibodies or CD32 small molecule inhibitors; CD33 inhibiting agents, including but not limited to anti-CD33 antibodies or CD33 small molecule inhibitors; CD44 inhibiting agents, including but not limited to anti-CD44 antibodies or CD44 small molecule inhibitors; CD45 inhibiting agents, including but not limited to anti-CD45 antibodies or CD45 small molecule inhibitors; CD66b inhibiting agents, including but not limited to anti-CD66b antibodies or CD66b small molecule inhibitors; CD18, or inhibiting agents, including but not limited to anti-CD18 antibodies or CD18 small molecule inhibitors; CD62L inhibiting agents, including but not limited to anti-CD62L antibodies or CD62L small molecule inhibitors; and Gr-1 inhibiting agents, including but not limited to anti-Gr-1 antibodies or Gr-1 small molecule inhibitors.

As used herein, the term “dendritic cell-depleting agent” refers to any agent that depletes or destroys dendritic cells in a subject and/or interferes with one or more dendrite functions. The dendritic cell-depleting agents can target any dendritic cell. Examples of dendritic cell-depleting agents include, e.g., agents that target phagocytic immune cell markers, e.g., PDCA1 inhibiting agents, including but not limited to anti-PDCA1 antibodies or PDCA1 small molecule inhibitors; and CD11c inhibiting agents, including but not limited to anti-CD11c antibodies or CD11c small molecule inhibitors.

As used herein, the term “inhibiting agent” refers to any compound capable of down-regulating, decreasing, reducing, suppressing, or inactivating the amount and/or activity of the targeted protein. Inhibiting agents can be proteins, oligo- and polypeptides, nucleic acids, genes, or chemical molecules. Suitable protein inhibitors can be, for example, monoclonal or polyclonal antibodies which bind to the targeted protein.

Any suitable CD115 inhibiting agent, including those known to those skilled in the art, in view of the present disclosure can be used in the invention. Examples of CD115 small molecule inhibitors include, e.g., pexidartinib (PLX-3397), BLZ-945, Linifanib (ABT-869), JNJ-28312141 (Johnson & Johnson), JNJ-40346527 (Johnson & Johnson), PLX7486 (Plexxikon), and ARRY-382 (Array BioPharma).

Any suitable anti-CD115 antibody, including those known to those skilled in the art, in view of the present disclosure can be used in the invention. Examples of commercial anti-CD115 antibodies include, e.g., AFS98 (Invitrogen or BioCell), 12-3A3-1B10 (Invitrogen), 6C7 (Bioss), cabiralizumab (FPA008), 25949-1-AP (Proteintech), 1G4 (Abnova), 3G12 (Abnova), 604B5 2E11 (Invitrogen), emactuzumab (RG-7155; Roche), AMG 820 (Amgen), IMC-CS4, and ROS8G11 (Invitrogen). In certain embodiments, the antibody or antigen-binding fragment thereof is AFS98 (e.g., BioCell BE0213), see also Oncogene. 1995;11(12):2469-2476.

Any suitable Ly6G inhibiting agent, including those known to those skilled in the art, in view of the present disclosure can be used in the invention.

Any suitable anti-Ly6G antibody, including those known to those skilled in the art, in view of the present disclosure can be used in the invention. Examples of commercial anti-Ly6G antibodies include, e.g., 1A8 (BioCell BP0075-1) and RB6-8C5 (ab25377).

Any suitable anti-CD177 antibodies or CD177 small molecule inhibitors, including those known to those skilled in the art, in view of the present disclosure can be used in the invention. Any suitable anti-CD14 antibodies or CD14 small molecule inhibitors, including those known to those skilled in the art, in view of the present disclosure can be used in the invention. Any suitable anti-CD15 antibodies or CD15 small molecule inhibitors, including those known to those skilled in the art, in view of the present disclosure can be used in the invention. Any suitable anti-CD11b antibodies or CD11b small molecule inhibitors, including those known to those skilled in the art, in view of the present disclosure can be used in the invention. Any suitable anti-CD16 antibodies or CD16 small molecule inhibitors, including those known to those skilled in the art, in view of the present disclosure can be used in the invention. Any suitable anti-CD32 antibodies or CD32 small molecule inhibitors, including those known to those skilled in the art, in view of the present disclosure can be used in the invention. Any suitable anti-CD33 antibodies or CD33 small molecule inhibitors, including those known to those skilled in the art, in view of the present disclosure can be used in the invention. Any suitable anti-CD44 antibodies or CD44 small molecule inhibitors, including those known to those skilled in the art, in view of the present disclosure can be used in the invention. Any suitable anti-CD45 antibodies or CD45 small molecule inhibitors, including those known to those skilled in the art, in view of the present disclosure can be used in the invention. Any suitable anti-CD66b antibodies or CD66b small molecule inhibitors, including those known to those skilled in the art, in view of the present disclosure can be used in the invention. Any suitable anti-CD18 antibodies or CD18 small molecule inhibitors, including those known to those skilled in the art, in view of the present disclosure can be used in the invention. Any suitable anti-CD62L antibodies or CD62L small molecule inhibitors, including those known to those skilled in the art, in view of the present disclosure can be used in the invention.

Intralipid and empty liposomes have been shown to interfere with one or more functions of monocytes and/or macrophages. See, e.g., Liu et al., Biochim Biophys Acta. 2013 Jun;1830(6):3447-53 and Saunders et al., Nano Lett. 2020 Jun 10;20(6):4264-4269. Pretreatment with intralipid or empty liposomes can effectively saturate monocyte/macrophage cells and prevent phagocytosis of a non-viral therapeutic agent. Any suitable intralipid or empty liposome known to those skilled in the art in view of the present disclosure can be used in the invention. Examples of intralipids and empty liposomes include, e.g., I141-100ML (Sigma Aldrich), 2B6063 (Baxter), and those described in Liu et al., Biochim Biophys Acta. 2013 Jun;1830(6):3447-53 and Saunders et al., Nano Lett. 2020 Jun 10;20(6):4264-4269.

Any suitable bisphosphonate known to those skilled in the art in view of the present disclosure can be used in the invention. Examples of bisphosphonates include, e.g., clodronate, pamidronate, ibandronate, alendronate, and zoledronate.

Other examples of “phagocyte-depleting agent” include, for example, palbociclib (Ibrance®; Pfizer), cromolyn sodium (Nasalcrom®; Bausch & Lomb), which are known to inhibit mast cells.

As used herein, the term “immunosuppressant” refers to any compound capable of slowing or halting immune system activity in a subject. Examples of immunosuppressants include, but are not limited to, a calcineurin inhibitor, such as, but not limited to, cyclosporine, ISA(TX) 247, tacrolimus or calcineurin, a target of rapamycin, such as, but not limited to, sirolimus, everolimus, FK778 or TAFA-93, an interleukin-2 α-chain blocker, such as, but not limited to, basiliximab and daclizumab, an inhibitor of inosine monophosphate dehydrogenase, such as mycophenolate mofetil, an inhibitor of dihydrofolic acid reductase, such as, but not limited to, methotrexate, an immunosuppressive antimetabolite, such as, but not limited to, azathioprine, a JAK inhibitor, such as, but not limited to ruxolitinib, a cytokine inhibitor, such as, not but limited to an anti-cytokine antibody, such as, but not limited to siltuximab, a cGAS-STING inhibitor, such as, but not limited to H-151, or a steroid.

As used herein, the term “steroid” is used to encompass both corticosteroids and glucocorticosteroids. In certain embodiments, the steroid can be a corticosteroid. Any suitable corticosteroid known to those skilled in the art in view of the present disclosure can be used in the invention. As used herein, the term “steroid” refers to a chemical substance comprising three cyclohexane rings and a cyclopentane ring. The rings are arranged to form tetracyclic cyclopentaphenanthrene, i.e., gonane. As used herein, the term “corticosteroid” refers to a class of steroid hormones that are produced in the adrenal cortex or produced synthetically. Corticosteroids are involved in a wide range of physiologic systems such as stress response, immune response and regulation of inflammation, carbohydrate metabolism, protein catabolism, blood electrolyte levels, and behavior. Corticosteroids are generally grouped into four classes, based on chemical structure. Group A corticosteroids (short to medium acting glucocorticoids) include hydrocortisone, hydrocortisone acetate, cortisone acetate, tixocortol pivalate, prednisolone, methylprednisolone, and prednisone. Group B corticosteroids include triamcinolone acetonide, triamcinolone alcohol, mometasone, amcinonide, budesonide, desonide, fluocinonide, fluocinolone acetonide, and halcinonide. Group C corticosteroids include betamethasone, betamethasone sodium phosphate, dexamethasone, dexamethasone sodium phosphate, and fluocortolone. Group D corticosteroids include hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate, and fluprednidene acetate. Non-limiting examples of corticosteroids include, aldosternone, beclomethasone, beclomethasone dipropionate, betametahasone, betametahasone-21-phosphate disodium, betametahasone valerate, budesonide, clobetasol, clobetasol propionate, clobetasone butyrate, clocortolone pivalate, cortisol, cortisteron, cortisone, deflazacort, dexamethasone, dexamethasone acetate, dexamethasone sodium phosphate, diflorasone diacetate, dihydroxycortison, flucinonide, fludrocortisones acetate, flumethasone, flunisolide, flucionolone acetonide, fluticasone furate, fluticasone propionate, halcinonide, halpmetasone, hydrocortisone, hydroconrtisone acetate, hydrocortisone succinate, 16α-hydroxyprednisolone, isoflupredone acetate, medrysone, methylprednisolone, prednacinolone, predricarbate, prednisolone, prednisolone acetate, prednisolone sodium succinate, prednisone, triamcinolone, triamcinolone, and triamcinolone diacetate. As used herein, the term “corticosteroid” can include, but is not limited to, the following generic and brand name corticosteroids: cortisone (CORTONE™ ACETATE™, ADRESON™, ALTESONA™, CORTELANT™, CORTISTAB™, CORTISYL™, CORTOGEN™, CORTONE™, SCHEROSON™); dexamethasone-oral (DECADRON ORAL™, DEXAMETH™, DEXONE™, HEXADROL-ORAL™, DEXAMETHASONE™ INTENSOL™, DEXONE 0.5™, DEXONE 0.75™, DEXONE 1.5 ™, DEXONE 4™); hydrocortisone-oral (CORTEF™, HYDROCORTONE™); hydrocortisone cypionate (CORTEF ORAL SUSPENSION™); methylprednisolone-oral (MEDROL-ORAL™); prednisolone-oral (PRELONE™, DELTA-CORTEF™, PEDIAPRED™, ADNISOLONE™, CORTALONE™, DELTACORTRIL™, DELTASOLONE™, DELTASTAB™, DI-ADRESON F™, ENCORTOLONE™, HYDROCORTANCYL™, MEDISOLONE™, METICORTELONE™, OPREDSONE™, PANAAFCORTELONE™, PRECORTISYL™, PRENISOLONA™, SCHERISOLONA™, SCHERISOLONE™); prednisone (DELTASONE™, LIQUID PRED™, METICORTENT™, ORASONE 1™, ORASONE 5™, ORASONE 10™, ORASONE 20™, ORASONE 50™, PREDNICEN-M™, PREDNISONE INTENSOL™, STERAPRED™, STERAPRED DS™, ADASONE™, CARTANCYL™, COLISONE™, CORDROL™, CORTAN™, DACORTIN™, DECORTIN™, DECORTISYL™, DELCORTIN™, DELLACORT™, DELTADOME™, DELTACORTENE™, DELTISONA™, DIADRESON™, ECONOSONE™, ENCORTON™, FERNISONE™, NISONA™, NOVOPREDNISONE™, PANAFCORT™, PANASOL™, PARACORT™, PARMENISON™, PEHACORT™, PREDELTIN™, PREDNICORT™, PREDNICOT™, PREDNIDIB™, PREDNIMENT™, RECTODELT™, ULTRACORTEN™, WINPRED™); triamcinoloneoral (KENACORT™, ARISTOCORT™, ATOLONE™, SHOLOG A™, TRAMACORT-D™, TRI-MED™, TRIAMCOT™, TRISTOPLEX™, TRYLONE D™, U-TRI-LONE™). In certain embodiments, a corticosteroid can be dexamethasone, prednisone, prednisolone, triamcinolone, clobetasol propionate, betamethasone valerate, betamethasone dipropionate, or mometasone furoate. Methods of synthesizing steroids and corticosteroids are well known in the art and such compounds are also commercially available, e.g. dexamethasone (Cat. No. D4902, Sigma-Aldrich; St. Louis, Mo.) and prednisone (Cat. No. P6254, Sigma-Aldrich; St. Louis, Mo.).

A corticosteroid, e.g., dexamethasone, can be delivered as free dexamethasone. Alternatively, a corticosteroid, e.g., dexamethasone, can be delivered by LNP, either as a separate LNP composition or as part of the same LNP composition as the therapeutic transgene.

### Therapeutic Compositions

In certain embodiments, the instant invention relates to a therapeutic composition comprising a phagocyte-depleting agent, according to the instant invention, for use in the treatment of a disease treated by gene therapy using a pharmaceutical composition comprising a non-viral vector comprising a transgene and a pharmaceutically acceptable carrier, according to the instant invention, in a patient in need thereof.

Any therapeutic agent or pharmaceutical composition of the instant invention can be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The form of the pharmaceutical compositions, the route of administration, the dosage and the regimen naturally depend upon the condition to be treated, the severity of the illness, the age, weight, and sex of the patient, etc.

Pharmaceutical compositions of the instant invention can be formulated for topical, oral, intranasal, parenteral, intraocular, intravenous, intramuscular or subcutaneous administration and the like.

Pharmaceutical compositions of the instant invention can contain vehicles which are pharmaceutically acceptable for a formulation capable of being injected. These can be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The doses used for the administration can be adapted as a function of various parameters, and in particular as a function of the mode of administration used, of the relevant pathology, or alternatively of the desired duration of treatment.

Reference to pharmaceutically acceptable salt indicate the salt at the provided amount is suitable for administration to a mammal, preferable a human.

In addition, other pharmaceutically acceptable forms include, e.g., tablets or other solids for oral administration; time release capsules; and any other form currently can be used.

Pharmaceutical compositions of the instant invention can comprise a further therapeutically active agent.

A pharmaceutical composition comprising a non-viral delivery nanoparticle and a non-viral vector comprising a therapeutic transgene, operably linked to a promoter, can be administered to a subject at any suitable dose. For example, a suitable dosage can be from about 0.01 mg/ml to about 10 mg/ml of the non-viral vector per kg body weight of a subject.

An antibody that targets a phagocytic immune cell marker can be administered to a subject at any suitable dose. For example, a suitable dosage can be from about 0.01 mg/kg to about 5 mg/kg body weight of a subject, wherein the dosage is administered in 1 to 10 total injections.

An anti-CD115 antibody can be administered to a subject at any suitable dose. For example, a suitable dosage can be from about 0.01 mg/kg to about 5 mg/kg body weight of a subject, wherein the dosage is administered in 1 to 10 total injections. In certain embodiments, the dose can be from about 0.01 mg/kg to about 0.1 mg/kg, about 0.1 mg/kg to about 0.2 mg/kg, about 0.2 mg/kg to about 0.3 mg/kg, about 0.3 mg/kg to about 0.4 mg/kg, about 0.4 mg/kg to about 0.5 mg/kg, about 0.5 mg/kg to about 0.6 mg/kg, about 0.6 mg/kg to about 0.7 mg/kg, about 0.7 mg/kg to about 0.8 mg/kg, about 0.8 mg/kg to about 0.9 mg/kg, about 0.9 mg/kg to about 1 mg/kg, about 1 mg/kg to about 1.5 mg/kg, about 1.5 mg/kg to about 2 mg/kg, about 2 mg/kg to about 2.5 mg/kg, about 2.5 mg/kg to about 3 mg/kg, about 3 mg/kg to about 3.5 mg/kg, about 3.5 mg/kg to about 4 mg/kg, about 4 mg/kg to about 4.5 mg/kg, about 4.5 mg/kg to about 5 mg/kg. In certain embodiments, the dose is less than about any of the following doses (in mg/kg): 5, 4.9, 4.8, 4.7, 4.6, 4.5, 4.4, 4.3, 4.2, 4.1, 4, 3.9, 3.8, 3.7, 3.6, 3.5, 3.4, 3.3, 3.2, 3.1, 3, 2.9, 2.8, 2.7, 2.6, 2.5, 2.4, 2.3, 2.2, 2.1, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, 0.1, 0.09, 0.08, 0.07, 0.06, 0.05, 0.04, 0.03, or 0.02. In certain embodiments, the dose is greater than about any of the following doses (in mg/kg): 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, or 4.9. That is, the dose can be any of a range of doses (in mg/ml) having an upper limit of 5, 4.9, 4.8, 4.7, 4.6, 4.5, 4.4, 4.3, 4.2, 4.1, 4, 3.9, 3.8, 3.7, 3.6, 3.5, 3.4, 3.3, 3.2, 3.1, 3, 2.9, 2.8, 2.7, 2.6, 2.5, 2.4, 2.3, 2.2, 2.1, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, 0.1, 0.09, 0.08, 0.07, 0.06, 0.05, 0.04, 0.03, or 0.02, and an independently selected lower limit of 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, or 4.9, wherein the lower limit is less than the upper limit.

An anti-Ly6G, anti-CD177, anti-CD14, anti-CD15 , anti-CD11b, anti-CD16, anti-CD32, anti-CD33, anti-CD44, anti-CD45, anti-CD66b, anti-CD18, or anti-CD62L antibody can be administered to a subject at any suitable dose. For example, a suitable dosage can be from about 0.01 mg/kg to about 5 mg/kg body weight of a subject, wherein the dosage is administered in 1 to 10 total injections. In certain embodiments, the dose can be from about 0.01 mg/kg to about 0.1 mg/kg, about 0.1 mg/kg to about 0.2 mg/kg, about 0.2 mg/kg to about 0.3 mg/kg, about 0.3 mg/kg to about 0.4 mg/kg, about 0.4 mg/kg to about 0.5 mg/kg, about 0.5 mg/kg to about 0.6 mg/kg, about 0.6 mg/kg to about 0.7 mg/kg, about 0.7 mg/kg to about 0.8 mg/kg, about 0.8 mg/kg to about 0.9 mg/kg, about 0.9 mg/kg to about 1 mg/kg, about 1 mg/kg to about 1.5 mg/kg, about 1.5 mg/kg to about 2 mg/kg, about 2 mg/kg to about 2.5 mg/kg, about 2.5 mg/kg to about 3 mg/kg, about 3 mg/kg to about 3.5 mg/kg, about 3.5 mg/kg to about 4 mg/kg, about 4 mg/kg to about 4.5 mg/kg, about 4.5 mg/kg to about 5 mg/kg. In certain embodiments, the dose is less than about any of the following doses (in mg/kg): 5, 4.9, 4.8, 4.7, 4.6, 4.5, 4.4, 4.3, 4.2, 4.1, 4, 3.9, 3.8, 3.7, 3.6, 3.5, 3.4, 3.3, 3.2, 3.1, 3, 2.9, 2.8, 2.7, 2.6, 2.5, 2.4, 2.3, 2.2, 2.1, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, 0.1, 0.09, 0.08, 0.07, 0.06, 0.05, 0.04, 0.03, or 0.02. In certain embodiments, the dose is greater than about any of the following doses (in mg/kg): 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, or 4.9. That is, the dose can be any of a range of doses (in mg/ml) having an upper limit of 5, 4.9, 4.8, 4.7, 4.6, 4.5, 4.4, 4.3, 4.2, 4.1, 4, 3.9, 3.8, 3.7, 3.6, 3.5, 3.4, 3.3, 3.2, 3.1, 3, 2.9, 2.8, 2.7, 2.6, 2.5, 2.4, 2.3, 2.2, 2.1, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, 0.1, 0.09, 0.08, 0.07, 0.06, 0.05, 0.04, 0.03, or 0.02, and an independently selected lower limit of 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, or 4.9, wherein the lower limit is less than the upper limit.

A small molecule inhibitor that targets a phagocytic immune cell marker can be administered to a subject at any suitable dose. For example, a suitable dosage can be from about 0.1 mg/kg to about 15 mg/kg body weight of a subject. In certain embodiments, the dose can be from about 0.1 mg/kg to about 1 mg/kg, about 1 mg/kg to about 2 mg/kg, about 2 mg/kg to about 3 mg/kg, about 3 mg/kg to about 4 mg/kg, about 4 mg/kg to about 5 mg/kg, about 5 mg/kg to about 6 mg/kg, about 6 mg/kg to about 7 mg/kg, about 7 mg/kg to about 8 mg/kg, about 8 mg/kg to about 9 mg/kg, about 9 mg/kg to about 10 mg/kg, about 10 mg/kg to about 11 mg/kg, about 11 mg/kg to about 12 mg/kg, about 12 mg/kg to about 13 mg/kg, about 13 mg/kg to about 14 mg/kg, about 14 mg/kg to about 15 mg/kg. In certain embodiments, the dose is less than about any of the following doses (in mg/kg): 15, 14, 13, 12, 11, 10, 9, 8, 7, 6, 5, 4, 3, 2, 1, 0.9, 0.8, 0.7, 0.6, 0.5, 0.4, 0.3, or 0.2. In certain embodiments, the dose is greater than about any of the following doses (in mg/kg): 0.1, 0.2, 0.3, 0.4, 0.5, 0.6, 0.7, 0.8, 0.9, 1, 2, 3, 4, 5, 6, 7, 8, 9, 10, 11, 12, 13, 14, or 15. That is, the dose can be any of a range of doses (in mg/ml) having an upper limit of 15, 14, 13, 12, 11, 10, 9, 8, 7, 6, 5, 4, 3, 2, 1, 0.9, 0.8, 0.7, 0.6, 0.5, 0.4, 0.3, or 0.2, and an independently selected lower limit of 0.1, 0.2, 0.3, 0.4, 0.5, 0.6, 0.7, 0.8, 0.9, 1, 2, 3, 4, 5, 6, 7, 8, 9, 10, 11, 12, 13, 14, or 15, wherein the lower limit is less than the upper limit.

A CD115 inhibitor such as pexidartinib can be administered to a subject at any suitable dose. For example, a suitable dosage can be from about 0.1 mg/kg to about 15 mg/g body weight of a subject. In certain embodiments, the dose can be from about 0.1 mg/kg to about 1 mg/kg, about 1 mg/kg to about 2 mg/kg, about 2 mg/kg to about 3 mg/kg, about 3 mg/kg to about 4 mg/kg, about 4 mg/kg to about 5 mg/kg, about 5 mg/kg to about 6 mg/kg, about 6 mg/kg to about 7 mg/kg, about 7 mg/kg to about 8 mg/kg, about 8 mg/kg to about 9 mg/kg, about 9 mg/kg to about 10 mg/kg, about 10 mg/kg to about 11 mg/kg, about 11 mg/kg to about 12 mg/kg, about 12 mg/kg to about 13 mg/kg, about 13 mg/kg to about 14 mg/kg, about 14 mg/kg to about 15 mg/kg. In certain embodiments, the dose is less than about any of the following doses (in mg/kg): 15, 14, 13, 12, 11, 10, 9, 8, 7, 6, 5, 4, 3, 2, 1, 0.9, 0.8, 0.7, 0.6, 0.5, 0.4, 0.3, or 0.2. In certain embodiments, the dose is greater than about any of the following doses (in mg/kg): 0.1, 0.2, 0.3, 0.4, 0.5, 0.6, 0.7, 0.8, 0.9, 1, 2, 3, 4, 5, 6, 7, 8, 9, 10, 11, 12, 13, 14, or 15. That is, the dose can be any of a range of doses (in mg/ml) having an upper limit of 15, 14, 13, 12, 11, 10, 9, 8, 7, 6, 5, 4, 3, 2, 1, 0.9, 0.8, 0.7, 0.6, 0.5, 0.4, 0.3, or 0.2, and an independently selected lower limit of 0.1, 0.2, 0.3, 0.4, 0.5, 0.6, 0.7, 0.8, 0.9, 1, 2, 3, 4, 5, 6, 7, 8, 9, 10, 11, 12, 13, 14, or 15, wherein the lower limit is less than the upper limit.

A bisphosphonate, e.g., clodronate, can be administered to a subject at any suitable dose. For example, a suitable dosage can be from about 0.1 mg/kg to about 15 mg/kg body weight of a subject. In certain embodiments, the dose can be from about 0.1 mg/kg to about 1 mg/kg, about 1 mg/kg to about 2 mg/kg, about 2 mg/kg to about 3 mg/kg, about 3 mg/kg to about 4 mg/kg, about 4 mg/kg to about 5 mg/kg, about 5 mg/kg to about 6 mg/kg, about 6 mg/kg to about 7 mg/kg, about 7 mg/kg to about 8 mg/kg, about 8 mg/kg to about 9 mg/kg, about 9 mg/kg to about 10 mg/kg, about 10 mg/kg to about 11 mg/kg, about 11 mg/kg to about 12 mg/kg, about 12 mg/kg to about 13 mg/kg, about 13 mg/kg to about 14 mg/kg, about 14 mg/kg to about 15 mg/kg. In certain embodiments, the dose is less than about any of the following doses (in mg/kg): 15, 14, 13, 12, 11, 10, 9, 8, 7, 6, 5, 4, 3, 2, 1, 0.9, 0.8, 0.7, 0.6, 0.5, 0.4, 0.3, or 0.2. In certain embodiments, the dose is greater than about any of the following doses (in mg/kg): 0.1, 0.2, 0.3, 0.4, 0.5, 0.6, 0.7, 0.8, 0.9, 1, 2, 3, 4, 5, 6, 7, 8, 9, 10, 11, 12, 13, 14, or 15. That is, the dose can be any of a range of doses (in mg/ml) having an upper limit of 15, 14, 13, 12, 11, 10, 9, 8, 7, 6, 5, 4, 3, 2, 1, 0.9, 0.8, 0.7, 0.6, 0.5, 0.4, 0.3, or 0.2, and an independently selected lower limit of 0.1, 0.2, 0.3, 0.4, 0.5, 0.6, 0.7, 0.8, 0.9, 1, 2, 3, 4, 5, 6, 7, 8, 9, 10, 11, 12, 13, 14, or 15, wherein the lower limit is less than the upper limit.

A corticosteroid, e.g., dexamethasone, can be administered to a subject at any suitable dose. For example, a suitable dosage can be from about 0.1 mg/kg to about 15 mg/kg body weight of a subject. In certain embodiments, the dose can be from about 0.1 mg/kg to about 1 mg/kg, about 1 mg/kg to about 2 mg/kg, about 2 mg/kg to about 3 mg/kg, about 3 mg/kg to about 4 mg/kg, about 4 mg/kg to about 5 mg/kg, about 5 mg/kg to about 6 mg/kg, about 6 mg/kg to about 7 mg/kg, about 7 mg/kg to about 8 mg/kg, about 8 mg/kg to about 9 mg/kg, about 9 mg/kg to about 10 mg/kg, about 10 mg/kg to about 11 mg/kg, about 11 mg/kg to about 12 mg/kg, about 12 mg/kg to about 13 mg/kg, about 13 mg/kg to about 14 mg/kg, about 14 mg/kg to about 15 mg/kg. In certain embodiments, the dose is less than about any of the following doses (in mg/kg): 15, 14, 13, 12, 11, 10, 9, 8, 7, 6, 5, 4, 3, 2, 1, 0.9, 0.8, 0.7, 0.6, 0.5, 0.4, 0.3, or 0.2. In certain embodiments, the dose is greater than about any of the following doses (in mg/kg): 0.1, 0.2, 0.3, 0.4, 0.5, 0.6, 0.7, 0.8, 0.9, 1, 2, 3, 4, 5, 6, 7, 8, 9, 10, 11, 12, 13, 14, or 15. That is, the dose can be any of a range of doses (in mg/ml) having an upper limit of 15, 14, 13, 12, 11, 10, 9, 8, 7, 6, 5, 4, 3, 2, 1, 0.9, 0.8, 0.7, 0.6, 0.5, 0.4, 0.3, or 0.2, and an independently selected lower limit of 0.1, 0.2, 0.3, 0.4, 0.5, 0.6, 0.7, 0.8, 0.9, 1, 2, 3, 4, 5, 6, 7, 8, 9, 10, 11, 12, 13, 14, or 15, wherein the lower limit is less than the upper limit.

### Methods

In certain embodiments, the instant invention relates to methods of delivering a transgene to a subject in need thereof comprising (a) administering to the subject a monocyte-and/or macrophage-depleting agent; and (b) administering to the subject a pharmaceutical composition comprising a non-viral vector comprising the transgene and a pharmaceutically acceptable carrier.

As used herein the term “a disease treated by gene therapy using a non-viral vector” denotes a disease wherein a polynucleotide (encoding at least one polypeptide or inhibitory nucleic acid) is delivered into the cell(s) of a patient as a drug to treat said disease (gene therapy).

In certain embodiments, the vector encodes at least one specific polypeptide or inhibitory nucleic acid useful to treat a disease. In certain embodiments, the vector encodes/comprises a therapeutic polynucleotide appropriate for treating a disease.

In certain embodiments, the vector encodes/comprises a therapeutic polynucleotide appropriate for treating a disease using gene therapy.

As used herein, the terms “patient” and “subject” interchangeably refer to an animal, typically a mammal, such as a rodent, a feline, a canine, and a primate. Particularly, a subject or patient according to the instant invention is a human.

As used herein, the term “treatment” or “treat” refers to both prophylactic or preventive treatment like gene therapy as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment can be administered to a subject having a medical disorder or who ultimately can acquire the disorder, in order to prevent, cure, delay the onset of, reduce the severity of, or ameliorate one or more symptoms of a disorder or recurring disorder, or in order to prolong the survival of a subject beyond that expected in the absence of such treatment.

Methods according to the instant invention can be performed in any suitable order unless otherwise indicated herein. In certain embodiments, a method can comprise first (a) administering to a subject a phagocyte-depleting agent, and then (b) administering to the subject a pharmaceutical composition comprising a non-viral vector comprising the transgene and a pharmaceutically acceptable carrier. In certain embodiments, (b) administering to the subject a pharmaceutical composition comprising a non-viral vector comprising the transgene and a pharmaceutically acceptable carrier, is performed between about 1 minute to about 1 year after (a) administering to the subject a phagocyte-depleting agent. In certain embodiments, (a) administering to the subject a phagocyte-depleting agent, and (b) administering to the subject a pharmaceutical composition comprising a non-viral vector comprising the transgene and a pharmaceutically acceptable carrier, are performed at about the same time.

In certain embodiments, a method can comprise first (a) administering to a subject a pharmaceutical composition comprising a non-viral vector comprising a transgene and a pharmaceutically acceptable carrier, and then (b) administering to the subject a phagocyte-depleting agent. In certain embodiments, (b) administering to the subject a phagocyte-depleting agent, is performed between about 1 minute to about 1 year after (a) administering to a subject a pharmaceutical composition comprising a non-viral vector comprising the transgene and a pharmaceutically acceptable carrier. In certain embodiments, (a) administering to a subject a pharmaceutical composition comprising a non-viral vector comprising the transgene and a pharmaceutically acceptable carrier, and (b) administering to the subject a phagocyte-depleting agent, are performed at about the same time.

In certain embodiments, the phagocyte-depleting agent is administered between about one minute to about 1 hour, 2 hours, 3 hours, or 4 hours before the non-viral vector comprising the transgene and a pharmaceutically acceptable carrier is administered.

In certain embodiments, the non-viral vector comprising the transgene and a pharmaceutically acceptable carrier is administered between about one minute to about 1 hour, 2 hours, 3 hours, or 4 hours before the phagocyte-depleting agent is administered.

In certain embodiments, the phagocyte-depleting agent is administered at least one day before the non-viral vector is administered, optionally no more than 1 year before the non-viral vector is administered, such as no more than 52 weeks, 51 weeks, 50 weeks, 49 weeks, 48 weeks, 47 weeks, 46 weeks, 45 weeks, 44 weeks, 43 weeks, 42 weeks, 41 weeks, 40 weeks, 39 weeks, 38 weeks, 37 weeks, 36 weeks, 35 weeks, 34 weeks, 33 weeks, 32 weeks, 31 weeks, 30 weeks, 29 weeks, 28 weeks, 27 weeks, 26 weeks, 25 weeks, 24 weeks, 23 weeks, 22 weeks, 21 weeks, 20 weeks, 19 weeks, 18 weeks, 17 weeks, 16 weeks, 15 weeks, 14 weeks, 13 weeks, 12 weeks, 11 weeks, 10 weeks, 9 weeks, 8 weeks, 7 weeks, 6 weeks, 5 weeks, 4 weeks, 3 weeks, 2 weeks, 1 week, 6 days, 5 days, 4 days, 3 days, or 2 days before the non-viral vector is administered.

A non-viral vector comprising a heterologous polynucleotide can be administered to a subject any number of times. For example, a non-viral vector comprising a heterologous polynucleotide can be administered once, 2 to 15 times to a subject. In certain embodiments the non-viral vector is administered multiple times, for example, 1, 2, 3, 4, 5, 6, 7, 8, 9 or 10 times. The spacing between the times can vary, for example, every 1 hour, 6 hours, 24 hours, 1-7 days, or 2-10 weeks; or a combination thereof.

A non-viral vector comprising a heterologous polynucleotide can be administered to a subject for any duration of time on a regular basis, such as consecutive days, or alternating days, or an irregular basis. In certain embodiments, a non-viral vector comprising a heterologous polynucleotide is administered from about 1 to 52 weeks, after administration of an immune cell modulator.

A phagocyte-depleting agent can be administered to a subject any number of times. For example, a phagocyte-depleting agent can be administered once, 2 to 5 times, 2 to 10 times, 2 to 15 times to a subject, per each administration of a non-viral vector comprising a heterologous polynucleotide.

An anti-CD115 antibody can be administered to a subject any number of times. For example, an anti-CD115 antibody can be administered once, 2 to 5 times, 2 to 10 times, 2 to 15 times to a subject, per each administration of a non-viral vector comprising a heterologous polynucleotide.

An anti-CD115 antibody can be administered to a subject for any duration of time on a regular basis, such as consecutive days, or alternating days, or an irregular basis. In certain embodiments, an anti-CD115 antibody is administered from about 1 to 52 weeks before administration of a non-viral vector.

A small molecule CD115 inhibitor can be administered to a subject any number of times. For example, a small molecule CD115 inhibitor can be administered once, 2 to 5 times, 2 to 10 times, 2 to 15 times to a subject, per each administration of a non-viral vector comprising a heterologous polynucleotide.

A small molecule CD115 inhibitor can be administered to a subject for any duration of time on a regular basis, such as consecutive days, or alternating days, or an irregular basis. In certain embodiments, a small molecule CD115 inhibitor is administered from about 1 to 52 weeks before administration of a non-viral vector.

An anti-Ly6G, anti-CD177, anti-CD14, anti-CD15 , anti-CD11b, anti-CD16, anti-CD32, anti-CD33, anti-CD44, anti-CD45, anti-CD66b, anti-CD18, or anti-CD62L antibody can be administered to a subject any number of times. For example, an anti-Ly6G, anti-CD177, anti-CD14, anti-CD15 , anti-CD11b, anti-CD16, anti-CD32, anti-CD33, anti-CD44, anti-CD45, anti-CD66b, anti-CD18, or anti-CD62L antibody can be administered once, 2 to 5 times, 2 to 10 times, 2 to 15 times to a subject, per each administration of a non-viral vector comprising a heterologous polynucleotide.

An anti-Ly6G, anti-CD177, anti-CD14, anti-CD15, anti-CD11b, anti-CD16, anti-CD32, anti-CD33, anti-CD44, anti-CD45, anti-CD66b, anti-CD18, or anti-CD62L antibody can be administered to a subject for any duration of time on a regular basis, such as consecutive days, or alternating days, or an irregular basis. In certain embodiments, an anti-Ly6G, anti-CD177, anti-CD14, anti-CD15 , anti-CD11b, anti-CD16, anti-CD32, anti-CD33, anti-CD44, anti-CD45, anti-CD66b, anti-CD18, or anti-CD62L antibody is administered from about 1 to 52 weeks before administration of a non-viral vector.

A bisphosphonate, e.g., clodronate, can be administered to a subject any number of times. For example, a bisphosphonate, e.g., clodronate, can be administered once, 2 to 5 times, 2 to 10 times, 2 to 15 times to a subject, per each administration of a non-viral vector comprising a heterologous polynucleotide.

A bisphosphonate, e.g., clodronate, can be administered to a subject for any duration of time on a regular basis, such as consecutive days, or alternating days, or an irregular basis. In certain embodiments, a bisphosphonate, e.g., clodronate, is administered from about 1 to 52 weeks before administration of a non-viral vector.

A corticosteroid, e.g., dexamethasone, can be administered to a subject any number of times. For example, a corticosteroid, e.g., dexamethasone, can be administered once, 2 to 5 times, 2 to 10 times, 2 to 15 times to a subject, per each administration of a non-viral vector comprising a heterologous polynucleotide.

A corticosteroid, e.g., dexamethasone, can be administered to a subject for any duration of time on a regular basis, such as consecutive days, or alternating days, or an irregular basis. In certain embodiments, a corticosteroid, e.g., dexamethasone, is administered from about 1 to 52 weeks before administration of a non-viral vector.

The non-viral vector comprising a heterologous polynucleotide can be administered alone or in a combination. In certain embodiments, the non-viral vector is administered to a subject separately from the at least one immune cell modulator, e.g. phagocyte-depleting agent, bisphosphonate; and/or immunosuppressant. In certain embodiments, the non-viral vector is administered to a subject in combination with the at least one immune cell modulator, e.g. phagocyte-depleting agent, bisphosphonate, and/or immunosuppressant. In certain embodiments, the bisphosphonate and/or immunosuppressant is included within the non-viral delivery particle, e.g., is encapsulated within a lipid nanoparticle, a polymer nanoparticle, a protein-based nanoparticle, or a peptide cage, along with the non-viral vector.

In certain embodiments, a mixture of at least one immune cell modulator, e.g. phagocyte-depleting agent, bisphosphonate, and/or immunosuppressant, is administered to a subject, one or more times. In certain embodiments, two or more immune cell modulators are administered to a subject, one or more times.

In certain embodiments, administration of at least one immune cell modulator to a subject reduces the dose of a non-viral vector comprising a heterologous polynucleotide required to be effective for treatment of a subject. In certain embodiments, administration of at least one immune cell modulator to a subject allows for administration of an increased dose of a non-viral vector comprising a heterologous polynucleotide.

Doses can vary and depend upon the type, onset, progression, severity, frequency, duration, or probability of the disease to which treatment is directed, the clinical endpoint desired, previous or simultaneous treatments, the general health, age, gender, race or immunological competency of the subject and other factors that will be appreciated by the skilled artisan. The dose amount, number, frequency or duration can be proportionally increased or reduced, as indicated by any adverse side effects, complications or other risk factors of the treatment or therapy and the status of the subject. The skilled artisan will appreciate the factors that can influence the dosage and timing required to provide an amount sufficient for providing a therapeutic or prophylactic benefit.

The dose to achieve a therapeutic effect, e.g., the dose in dsDNA mg per kilogram of body weight (mg/kg), will vary based on several factors including, but not limited to: route of administration, the level of heterologous polynucleotide expression required to achieve a therapeutic effect, the specific disease treated, any host immune response to the non-viral vector, any host immune response to the heterologous polynucleotide or expression product, and the stability of the protein, peptide, or nucleic acid expressed. One skilled in the art can determine a non-viral vector genome dose range to treat a patient having a particular disease or disorder based on the aforementioned factors, as well as other factors.

An “effective amount” or “sufficient amount” refers to an amount that provides, in single or multiple doses, alone or in combination, with one or more other compositions, treatments, protocols, or therapeutic regimens agents, a detectable response of any duration of time (long or short term), an expected or desired outcome in or a benefit to a subject of any measurable or detectable degree or for any duration of time (e.g., for minutes, hours, days, months, years, or cured). The doses of an “effective amount” or “sufficient amount” for treatment (e.g., to ameliorate or to provide a therapeutic benefit or improvement) typically are effective to provide a response to one, multiple or all adverse symptoms, consequences or complications of the disease, one or more adverse symptoms, disorders, illnesses, pathologies, or complications, for example, caused by or associated with the disease, to a measurable extent, although decreasing, reducing, inhibiting, suppressing, limiting or controlling progression or worsening of the disease is a satisfactory outcome.

In certain embodiments, the methods of the instant invention produce a therapeutically effective non-viral gene therapy in a subject in need thereof, wherein the therapeutically effective non-viral gene therapy is characterized by expression of the therapeutic protein or nucleic acid and/or minimal or absent undesirable immune response induced by the non-viral vector.

In certain embodiments, “expression of the therapeutic protein or nucleic acid” refers to an adequate level of protein or nucleic acid to result in a therapeutic effect, as discussed herein.

In certain embodiments, a method according to the instant invention results in expression of the transgene in the subject.

In certain embodiments, a method according to the instant invention can result in expression or activity of a therapeutic protein at a therapeutically effective level.

In certain embodiments, a method according to the instant invention can result in expression or activity of a therapeutic protein at a level that is at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100% of normal expression of the heterologous protein.

In certain embodiments, a method according to the instant invention can result in reduction of expression or activity of a protein targeted by a therapeutic nucleic acid.

In certain embodiments, a method according to the instant invention can result in reduction of expression or activity of a protein targeted by a therapeutic nucleic acid by at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100% of normal expression of the target protein.

Non-limiting examples of a biological sample from a subject that can be analyzed include whole blood, serum, plasma, the like, and a combination thereof. A biological sample can be devoid of cells, or can include cells (e.g., red blood cells, platelets and/or lymphocytes).

Any suitable method for measuring expression of a therapeutic protein or nucleic acid known to those skilled in the art in view of the present disclosure can be used in the invention. Exemplary methods for analyzing and measuring heterologous protein or nucleic acid expression levels in a biological sample include, e.g., ELISA.

As used herein, the term “immune response” refers to a response by the immune system of a subject. For example, immune responses include, but are not limited to, a detectable alteration (e.g., increase) in Toll receptor activation, lymphokine (e.g., cytokine (e.g., Th1 or Th2 type cytokines) or chemokine) expression and/or secretion, macrophage activation, dendritic cell activation, T cell activation (e.g., CD4+ or CD8+ T cells), NK cell activation, and/or B cell activation (e.g., antibody generation and/or secretion). Additional examples of immune responses include binding of an immunogen (e.g., antigen (e.g., immunogenic polypeptide)) to an MHC molecule and inducing a cytotoxic T lymphocyte (“CTL”) response, inducing a B cell response (e.g., antibody production), and/or T-helper lymphocyte response, and/or a delayed type hypersensitivity (DTH) response against the antigen from which the immunogenic polypeptide is derived, expansion (e.g., growth of a population of cells) of cells of the immune system (e.g., T cells, B cells (e.g., of any stage of development (e.g., plasma cells), and increased processing and presentation of antigen by antigen presenting cells. An immune response can be to immunogens that the subject's immune system recognizes as foreign (e.g., non-self antigens from microorganisms (e.g., pathogens), or self-antigens recognized as foreign). Thus, it is to be understood that, as used herein, “immune response” refers to any type of immune response, including, but not limited to, innate immune responses (e.g., activation of Toll receptor signaling cascade) cell-mediated immune responses (e.g., responses mediated by T cells (e.g., antigen- specific T cells) and non-specific cells of the immune system) and humoral immune responses (e.g., responses mediated by B cells (e.g., via generation and secretion of antibodies into the plasma, lymph, and/or tissue fluids). The term “immune response” is meant to encompass all aspects of the capability of a subject's immune system to respond to antigens and/or immunogens.

As used herein, “minimal or absent undesirable immune response” induced by the non-viral vector refers to a safe or therapeutically tolerable immune response within a human subject. The undesirable immune response induced by the vector is distinct from a desirable immune response in the subject that is induced by an antigen encoded by a non-viral vector.

In certain embodiments, the safe or therapeutically tolerable immune response relates to cytokine release levels in a subject upon administration of the non-viral vector comprising the heterologous polynucleotide.

In certain embodiments, methods described herein decrease incidence of toxic cytokine release or “cytokine release syndrome” (CRS) or “severe cytokine release syndrome” (sCRS) or “cytokine storm” that can occur in a subject.

A person skilled in the art would appreciate that decreasing toxic cytokine release or toxic cytokine levels comprises decreasing or inhibiting production of toxic cytokine levels in a subject, or inhibiting or reducing the incidence of cytokine release syndrome or a cytokine storm in a subject. In certain embodiments, the toxic cytokines comprise pro-inflammatory cytokines. In certain embodiments, pro-inflammatory cytokines comprise IL-6, IFN-γ, IL-1β, or TNF-α, or any combination thereof.

In certain embodiments, cytokine release syndrome is characterized by elevated levels of several inflammatory cytokines and adverse physical reactions in a subject such as low blood pressure, high fever and shivering. In certain embodiments, CRS is characterized by elevated levels of IL-6, IFN-γ, IL-1β, or TNF-α, or any combination thereof.

In certain embodiments, measurement of cytokine levels or concentration, as an indicator of cytokine storm, can be expressed as fold increase, percent (%) increase, net increase or rate of change in cytokine levels or concentration. In certain embodiments, absolute cytokine levels or concentrations above a certain level or concentration can be an indication of a subject undergoing or about to experience a cytokine storm. In certain embodiments, absolute cytokine levels or concentration at a certain level or concentration, for example a level or concentration normally found in a control subject not undergoing non-viral gene therapy, can be an indication of a method for inhibiting or reducing the incidence of a cytokine storm in a subject undergoing non-viral gene therapy.

A person skilled in the art would appreciate that the term “cytokine level” can encompass a measure of concentration, a measure of fold change, a measure of percent (%) change, or a measure of rate change. Further, the methods for measuring cytokines in blood, saliva, serum, urine, and plasma are well known in the art.

In certain embodiments, despite the recognition that cytokine storm is associated with elevation of several inflammatory cytokines, INF-γ levels can be used as a common measure of cytokine storm and/or as a common measure of the effectiveness of a treatment for cytokine storms. In certain embodiments, despite the recognition that cytokine storm is associated with elevation of several inflammatory cytokines, IL-6 levels can be used as a common measure of cytokine storm and/or as a common measure of the effectiveness of a treatment for cytokine storms. A person skilled in the art would appreciate that other cytokines can be used as markers of a cytokine storm, for example TNF-α, IB-1α, IL-8, or IL-13.

Levels of cytokines in a subject can be analyzed, measured or determined before and/or after administration of the non-viral vector comprising a heterologous polynucleotide. Levels of cytokines in a subject can also be analyzed, measured or determined before and/or after the administration of the immune cell modulator. Levels of cytokines in a subject can also be analyzed or measured multiple times, before and/or after administration of the non-viral vector comprising a heterologous polynucleotide as well as before and/or after administration of the immune cell modulator.

Any suitable method for measuring levels of cytokines known to those skilled in the art in view of the present disclosure can be used in the invention. Exemplary methods for analyzing and measuring cytokine levels in a biological sample include, e.g., mesoscale delivery platform (MSD).

An effective amount or a sufficient amount can but need not be provided in a single administration, can require multiple administrations, and, can but need not be, administered alone or in combination with another composition (e.g., agent), treatment, protocol or therapeutic regimen. For example, the amount can be proportionally increased as indicated by the need of the subject, type, status and severity of the disease treated or side effects (if any) of treatment. In addition, an effective amount or a sufficient amount need not be effective or sufficient if given in single or multiple doses without a second composition (e.g., another drug or agent), treatment, protocol or therapeutic regimen, since additional doses, amounts or duration above and beyond such doses, or additional compositions (e.g., drugs or agents), treatments, protocols or therapeutic regimens can be included in order to be considered effective or sufficient in a given subject. Amounts considered effective also include amounts that result in a reduction of the use of another treatment, therapeutic regimen or protocol, such as administration of recombinant GAA for treatment of a lysosomal storage disease (e.g., Pompe disease), or administration of a recombinant clotting factor protein (e.g., FVIII or FIX) for treatment of a clotting disorder (e.g., hemophilia A (HemA) or hemophilia B (HemB)).

For Pompe disease, an effective amount would be an amount of GAA that inhibits or reduces glycogen production or accumulation, enhances or increases glycogen degradation or removal, reduces lysosomal alterations in tissues of the body of a subject, or improves muscle tone and/or muscle strength and/or respiratory function in a subject, for example. Effective amounts can be determined, for example, by ascertaining the kinetics of GAA uptake by myoblasts from plasma. Myoblasts GAA uptake rates (K uptake) of about 141-147 nM can appear to be effective (see, e.g., Maga et al., J. Biol. Chem. 2012) In animal models, GAA activity levels in plasma of greater than about 1,000 nmol/hr/mL, for example, about 1,000 to about 2,000 nmol/hr/mL have been observed to be therapeutically effective.

For HemA and HemB, generally speaking, it is believed that, in order to achieve a therapeutic effect, a blood coagulation factor concentration that is greater than 1% of factor concentration found in a normal individual is needed to change a severe disease phenotype to a moderate one. A severe phenotype is characterized by joint damage and life-threatening bleeds. To convert a moderate disease phenotype into a mild one, it is believed that a blood coagulation factor concentration greater than 5% of normal is needed.

FVIII and FIX levels in normal humans are about 150-200 ng/mL plasma, but can be less (e.g., range of about 100-150 ng/mL) or greater (e.g., range of about 200-300 ng/mL) and still considered normal, due to functional clotting as determined, for example, by an activated partial thromboplastin time (aPTT) one-stage clotting assay. Thus, a therapeutic effect can be achieved such that the total amount of FVIII or FIX in the subject/human is greater than 1% of the FVIII or FIX present in normal subjects/humans, e.g., 1% of 100-300 ng/mL.

The composition can be administered to a subject as a combination composition, or administered separately, such as concurrently or in series or sequentially (prior to or following) delivery or administration of a non-viral vector comprising a heterologous polynucleotide. The instant invention provides combinations in which a method or use of the instant invention is in a combination with any compound, agent, drug, therapeutic regimen, treatment protocol, process, remedy or composition, set forth herein or known to one of skill in the art. The compound, agent, drug, therapeutic regimen, treatment protocol, process, remedy or composition can be administered or performed prior to, substantially contemporaneously with or following administration of a non-viral vector comprising a heterologous polynucleotide, to a subject.

Accordingly, the instant invention includes methods and uses that result in a reduced need or use of another compound, agent, drug, therapeutic regimen, treatment protocol, process, or remedy. For example, for a blood clotting disease, a method of treatment according to the instant invention has a therapeutic benefit if in a given subject a less frequent or reduced dose or elimination of administration of a recombinant clotting factor protein to supplement for the deficient or defective (abnormal or mutant) endogenous clotting factor in the subject. In another example, for a lysosomal storage disease, such as Pompe disease, a methods of treatment according to the instant invention has a therapeutic benefit even if a less frequent or reduced dose of a recombinant viral vector comprising GAA has been previously administered, or continues to be administered to a subject. Thus, reducing the need for, or the use of, another treatment or therapy is included in the instant invention.

An effective amount or a sufficient amount need not be effective in each and every subject treated, nor a majority of treated subjects in a given group or population. An effective amount or a sufficient amount means effectiveness or sufficiency in a particular subject, not a group or the general population. As is typical for such methods, some subjects will exhibit a greater response, or less or no response to a given treatment method or use.

The term “ameliorate” means a detectable or measurable improvement in a subject's disease or symptom thereof, or an underlying cellular response. A detectable or measurable improvement includes a subjective or objective decrease, reduction, inhibition, suppression, limit or control in the occurrence, frequency, severity, progression, or duration of the disease, or complication caused by or associated with the disease, or an improvement in a symptom or an underlying cause or a consequence of the disease, or a reversal of the disease. For Pompe, an effective amount would be an amount that inhibits or reduces glycogen production or accumulation, enhances or increases glycogen degradation or removal, improves muscle tone and/or muscle strength and/or respiratory function, for example. For HemA or HemB, an effective amount would be an amount that reduces frequency or severity of acute bleeding episodes in a subject, for example, or an amount that reduces clotting time as measured by a clotting assay, for example.

Accordingly, pharmaceutical compositions of the instant invention include compositions wherein the active ingredients are contained in an effective amount to achieve the intended therapeutic purpose. Determining a therapeutically effective dose is well within the capability of a skilled medical practitioner using techniques and guidance known in the art and using the teachings provided herein.

Therapeutic doses will depend on, among other factors, the age and general condition of the subject, the severity of the aberrant phenotype, and the strength of the control sequences regulating expression levels. Thus, a therapeutically effective amount in humans will fall in a relatively broad range that can be determined by a medical practitioner based on the response of an individual patient to a vector-based treatment.

Compositions such as pharmaceutical compositions can be delivered to a subject, so as to allow transgene expression and optionally production of encoded protein. In certain embodiments, pharmaceutical compositions comprising sufficient genetic material to enable a subject to produce a therapeutically effective amount of a therapeutic protein or nucleic acid.

In certain embodiments, a therapeutic effect in a subject is sustained for a desirable period of time. Accordingly, in certain embodiments, a non-viral vector provides a therapeutic effect.

Compositions can be administered in any sterile, biocompatible pharmaceutical carrier, including, but not limited to, saline, buffered saline, dextrose, and water. The compositions can be administered to a patient alone, or in combination with other agents, which influence dosage amount, administration frequency and/or therapeutic efficacy.

Methods and uses of the instant invention include delivery and administration systemically, regionally or locally, or by any route, for example, by injection or infusion. Delivery of the compositions in vivo can generally be accomplished via injection using a conventional syringe, although other delivery methods such as convection-enhanced delivery are envisioned (see, e.g., U.S. Pat. No. 5,720,720). For example, compositions can be delivered subcutaneously, epidermally, intradermally, intrathecally, intraorbitally, intramucosally, intraperitoneally (IP), intravenously (IV), intra-pleurally, intraarterially, orally, intrahepatically, via the portal vein, or intramuscularly. Other modes of administration include oral and pulmonary administration, suppositories, and transdermal applications. A clinician specializing in the treatment of patients can determine the optimal route for administration of the compositions based on a number of criteria, including, but not limited to: the condition of the patient and the purpose of the treatment.

The compound, agent, drug, treatment or other therapeutic regimen or protocol can be administered as a combination composition, or administered separately, such as concurrently or in series or sequentially (prior to or following) delivery or administration of a non-viral vector. The instant invention therefore provides combinations in which a method of treatment according to the instant invention is in a combination with any compound, agent, drug, therapeutic regimen, treatment protocol, process, remedy or composition, set forth herein or known to one of skill in the art. The compound, agent, drug, therapeutic regimen, treatment protocol, process, remedy or composition can be administered or performed prior to, substantially contemporaneously with or following administration of a non-viral vector to a patient according to the instant invention.

Methods according to the instant invention are applicable to both loss of function and gain and function genetic defects. The term “loss-of-function” in reference to a genetic defect as used herein, refers to any mutation in a gene in which the protein encoded by said gene (i.e., the mutant protein) exhibits either a partial or a full loss of function that is normally associated with the wild-type protein. The term “gain-of-function” in reference to a genetic defect as used herein, refers to any mutation in a gene in which the protein encoded by said gene (i.e., the mutant protein) acquires a function not normally associated with the protein (i.e., the wild type protein) causes or contributes to a disease or disorder. The gain-of-function mutation can be a deletion, addition, or substitution of a nucleotide or nucleotides in the gene, which gives rise to the change in the function of the encoded protein. In certain embodiments, the gain-of-function mutation changes the function of the mutant protein or causes interactions with other proteins. In certain embodiments, the gain-of-function mutation causes a decrease in or removal of normal wild-type protein, for example, by interaction of the altered, mutant protein with said normal, wild-type protein.

Diseases and disorders that can be treated by methods according to the instant invention include, for example and without limitation, lung disease (e.g., cystic fibrosis), a bleeding disorder (e.g., hemophilia A or hemophilia B with or without inhibitors), thalassemia, a blood disorder (e.g., anemia), Alzheimer's disease, Parkinson's disease, Huntington's disease, amyotrophic lateral sclerosis (ALS), epilepsy, a lysosomal storage disease (e.g., aspartylglucosaminuria, Batten disease, late infantile neuronal ceroid lipofuscinosis type 2 (CLN2), cystinosis, Fabry disease, Gaucher disease types I, II, and III, glycogen storage disease II (Pompe disease), GM2-gangliosidosis type I (Tay Sachs disease), GM2-gangliosidosis type II (Sandhoff disease), mucolipidosis types I (sialidosis type I and II), II (I-cell disease), III (pseudo-Hurler disease) and IV, mucopolysaccharide storage diseases (Hurler disease and variants, Hunter, Sanfilippo Types A,B,C,D, Morquio Types A and B, Maroteaux-Lamy and Sly diseases), Niemann-Pick disease types A/B, C1 and C2, and Schindler disease types I and II), hereditary angioedema (HAE), a copper or iron accumulation disorder (e.g., Wilson's or Menkes disease), lysosomal acid lipase deficiency, a neurological or neurodegenerative disorder, cancer, type 1 or type 2 diabetes, adenosine deaminase deficiency, a metabolic defect (e.g., glycogen storage diseases), a disease of solid organs (e.g., brain, liver, kidney, heart), or an infectious viral (e.g., hepatitis B and C, HIV, etc.), bacterial or fungal disease.

Glycogen storage disease type II, also called Pompe disease can be treated by methods according to the instant invention. Pompe disease is an autosomal recessive disorder caused by mutations in the gene encoding the lysosomal enzyme acid α-glucosidase (GAA), which catalyzes the degradation of glycogen. The resulting enzyme deficiency leads to pathological accumulation of glycogen and lysosomal alterations in all tissues of the body, resulting in cardiac, respiratory, and skeletal muscle dysfunction (van der Ploeg & Reuser, 2008).

Blood clotting disorders which can be treated by methods according to the instant invention, include, for example and without limitation, hemophilia A, hemophilia A with inhibitory antibodies, hemophilia B, hemophilia B with inhibitory antibodies, a deficiency in any coagulation Factor: VII, VIII, IX, X, XI, V, XII, II, von Willebrand factor, or a combined FV/FVIII deficiency, thalassemia, vitamin K epoxide reductase C1 deficiency or gamma-carboxylase deficiency.

Other diseases and disorders that can be treated by methods according to the instant invention include, for example and without limitation, anemia, bleeding associated with trauma, injury, thrombosis, thrombocytopenia, stroke, coagulopathy, disseminated intravascular coagulation (DIC); over-anticoagulation associated with heparin, low molecular weight heparin, pentasaccharide, warfarin, small molecule antithrombotics (i.e., FXa inhibitors), or a platelet disorder such as, Bernard Soulier syndrome, Glanzmann thrombasthenia, or storage pool deficiency.

Other diseases and disorders that can be treated by methods according to the instant invention include, for example and without limitation, proliferative diseases (cancers, tumors, dysplasias, etc.), Crigler-Najjar and metabolic diseases like metabolic diseases of the liver, Friedreich ataxia, infectious diseases, viral diseases (induced, e.g., by hepatitis B or C viruses, HIV, herpes, retroviruses, etc.), genetic diseases (cystic fibrosis, dystroglycanopathies, myopathies such as Duchenne muscular myopathy or dystrophy, myotubular myopathy, sickle-cell anemia, sickle cell disease, Fanconi's anemia, diabetes, amyotrophic lateral sclerosis (ALS), myotubularin myopathy, motor neuron diseases such as spinal muscular atrophy (SMA), spinobulbar muscular atrophy, or Charcot-Marie-Tooth disease, arthritis, severe combined immunodeficiencies (such as RS-SCID, ADA-SCID or X-SCID), Wiskott-Aldrich syndrome, X-linked thrombocytopenia, X-linked congenital neutropenia, chronic granulomatous disease, etc.), dotting factor deficiencies, cardiovascular disease (restenosis, ischemia, dyslipidemia, homozygous familial hypercholesterolemia, etc.), eye diseases such as retinitis pigmentosa, Leber congenital amaurosis, Leber hereditary optic neuropathy, and Stargardt disease; lysosomal storage diseases such as San Filippo syndrome; hyperbilirubinemia such as CN type I or II or Gilbert's syndrome, glycogen storage disease such as GSDI, GSDII (Pompe disease), GSDIII, GSDIV, GSDV, GSDVI, GSDVII, GSDVIII or lethal congenital glycogen storage disease of the heart.

In certain embodiments, the subject has a disease that affects or originates in the central nervous system (CNS). In certain embodiments, the disease is a neurodegenerative disease. Non-limiting examples of CNS or neurodegenerative disease include Alzheimer's disease, Huntington's disease, ALS, hereditary spastic hemiplegia, primary lateral sclerosis, spinal muscular atrophy, Kennedy's disease, a polyglutamine repeat disease, or Parkinson's disease. In certain embodiments, the disease is a psychiatric disease, an addition (e.g., to tobacco, alcohol, or drugs), epilepsy, Canavan's disease, or adrenoleukodystrophy. In certain embodiments, the CNS or neurodegenerative disease is a polyglutamine repeat disease such as, for example and without limitation, spinocerebellar ataxia (SCA1, SCA2, SCA3, SCA6, SCA7, or SCA17).

The instant invention can be used in human and veterinary medical applications. Suitable subjects therefore include mammals, such as humans, as well as non-human mammals. The term “subject” refers to an animal, typically a mammal, such as humans, non-human primates (apes, gibbons, gorillas, chimpanzees, orangutans, macaques), a domestic animal (dogs and cats), a farm animal (poultry such as chickens and ducks, horses, cows, goats, sheep, pigs), and experimental animals (mouse, rat, rabbit, guinea pig). Human subjects include fetal, neonatal, infant, juvenile and adult subjects. Subjects also include animal disease models, for example, mouse and other animal models of protein/enzyme deficiencies such as Pompe disease (loss of GAA), and glycogen storage diseases (GSDs) and others known to those of skill in the art.

### Kits

Further provided herein is a composition or combination, for example, as a package or kit, having (a) a pharmaceutical composition comprising a non-viral vector comprising a transgene and a pharmaceutically acceptable carrier; (b) a phagocyte-depleting agent; and (c) a label with instructions for performing the method described herein, wherein (a) and (b) are in separate or the same container. Any suitable phagocyte-depleting agent can be included in the combination or kit, including but not limited to CD115 inhibiting agents, such as an antibody or an antigen binding fragment thereof that specifically binds to CD115, or a small molecule inhibitor of CD115; Ly6G inhibiting agents, including but not limited to, anti-Ly6G antibodies or anti-Ly6G small molecule inhibitors; CD177 inhibiting agents, including but not limited to anti-CD177 antibodies or CD177 small molecule inhibitors; CD14 inhibiting agents, including but not limited to anti-CD14 antibodies or CD14 small molecule inhibitors; CD15 inhibiting agents, including but not limited to anti-CD15 antibodies or CD15 small molecule inhibitors; CD11b inhibiting agents, including but not limited to anti-CD11b antibodies or CD11b small molecule inhibitors; CD16 inhibiting agents, including but not limited to anti-CD16 antibodies or CD16 small molecule inhibitors; CD32 inhibiting agents, including but not limited to anti-CD32 antibodies or CD32 small molecule inhibitors; CD33 inhibiting agents, including but not limited to anti-CD33 antibodies or CD33 small molecule inhibitors; CD44 inhibiting agents, including but not limited to anti-CD44 antibodies or CD44 small molecule inhibitors; CD45 inhibiting agents, including but not limited to anti-CD45 antibodies or CD45 small molecule inhibitors; CD66b inhibiting agents, including but not limited to anti-CD66b antibodies or CD66b small molecule inhibitors; CD18, or inhibiting agents, including but not limited to anti-CD18 antibodies or CD18 small molecule inhibitors; CD62L inhibiting agents, including but not limited to anti-CD62L antibodies or CD62L small molecule inhibitors; intralipids; empty liposomes; and bisphosphonates.

In certain embodiments, the pharmaceutical composition comprises (a) a non-viral delivery nanoparticle; and (b) the non-viral vector, wherein the non-viral vector comprises the transgene operably linked to a promoter.

In certain embodiments of the instant invention have been described. Nevertheless, one skilled in the art, without departing from the spirit and scope of the instant invention, can make various changes and modifications of the instant invention to adapt it to various usages and conditions. Accordingly, the following examples are intended to illustrate but not limit the scope of the instant invention claimed in any way.

### EXAMPLES

### Example 1—Immune Cell Depletion with Anti-CD115 Improved Transgene Expression

The effects of immune depletion on cytokine release and on transgene expression were studied using an anti-CD115 antibody (BioXcell, clone AFS98), which targets monocytes and macrophages; using an anti-Ly6G antibody (BioXcell, clone 1A8), which targets myeloid-derived cells; using large ODNs, e.g., ODN oligo formulated in LNPs with a size of ˜200 nm, which inhibit TLR-9, AIM2, and other innate immune sensors; or using H-151, which is a small molecule stimulator of interferon genes (STING) inhibitor.

10 or 50 μg of transgene DNA plasmid (nano-plasmid encoding human coagulation factor IX (hFIX)) formulated in lipid nanoparticle (LNP, purchased from Precision Nanosystems) delivery vehicles (DNA-LNP) (DNA was purchased from Nature Tech) was dosed in mice (n=5, balb/c from Jackson Laboratory) by intravenous (IV) administration alone or with intraperitoneal (IP) administration of anti-CD115 antibody, anti-Ly6G antibody, or H-151 (purchased from InvivoGen), or 50 μg large ODN (purchased from InvivoGen), as outlined in Table 2.

TABLE 2
Experimental Design

| Group | Group name | Day −2 | Day −1 | Day 0 | Day 1 | Day 2 | Day 3 |
|---|---|---|---|---|---|---|---|
| 1 | DNA-LNP 50 μg | | | 50 μg DNA-LNP (IV) | | | |
| 2 | DNA-LNP 50 μg + | 300 μg | | 300 μg CD115 (IP) + | | 300 μg | |
| | anti-CD115 | CD115 (IP) | | 50 μg DNA-LNP (IV) | | CD115 IP) | |
| 3 | DNA-LNP 50 μg + | 400 μg | | 400 μg LY6G (IP) + | | | |
| | anti-LY6G | Ly6G (IP) | | 50 μg DNA-LNP (IV) | | | |
| 4 | DNA-LNP 50 μg + | | | 50 μg ODN large | | | |
| | ODN large 50 μg | | | (IV) + 50 μg | | | |
| | | | | DNA-LNP (IV) | | | |
| 5 | DNA-LNP 50 μg + | 200 μg | 200 μg | 200 μg H151 (IP) + | 200 μg | 200 μg | 200 μg |
| | H151 | H151 (IP) | H151 (IP) | 50 μg DNA-LNP (IV) | H151 (IP) | H151 (IP) | H151 (IP) |
| 6 | DNA-LNP 10 μg | | | 10 μg DNA-LNP (IV) | | | |
| 7 | DNA-LNP 10 μg + | 300 μg | | 300 μg CD115 (IP) + | | 300 μg | |
| | anti-CD115 | CD115 (IP) | | 10 μg DNA-LNP (IV) | | CD115 IP) | |
| 8 | DNA-LNP 10 μg + | 400 μg | | 400 μg LY6G (IP) + | | | |
| | anti-LY6G | Ly6G (IP) | | 10 μg DNA-LNP (IV) | | | |
| 9 | DNA-LNP 10 μg + | | | 10 μg ODN large (IV) + | | | |
| | ODN large 10 μg | | | 10 μg DNA-LNP (IV) | | | |
| 10 | DNA-LNP 10 μg + | 200 μg | 200 μg | 200 μg H151 (IP) + | 200 μg | 200 μg | 200 μg |
| | H151 | H151 (IP) | H151 (IP) | 10 μg DNA-LNP (IV) | H151 (IP) | H151 (IP) | H151 (IP) |

### Immune Response

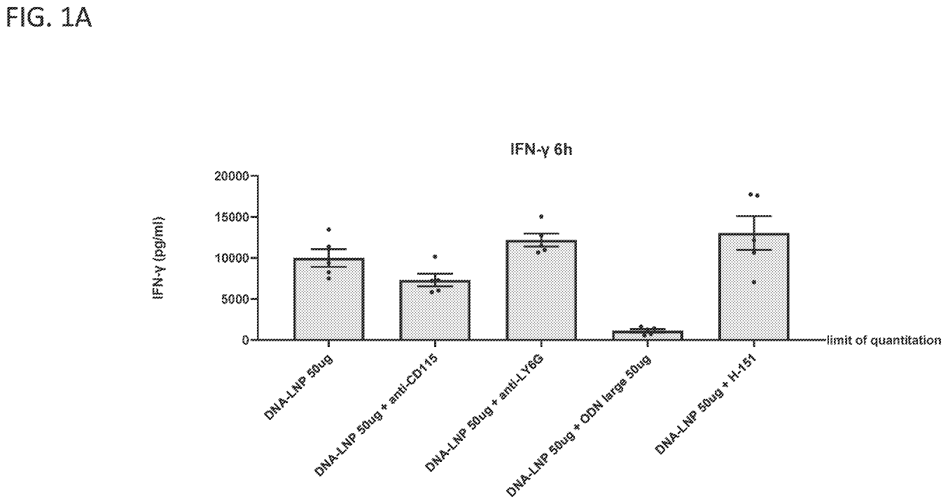

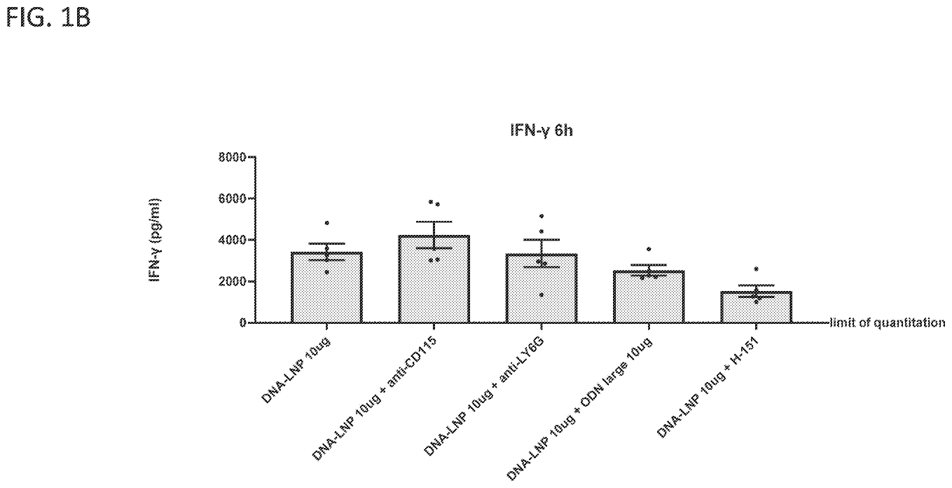

6-hour post-dose plasma samples were analyzed for a panel of pro-inflammatory cytokines using MSD: IL-6, IFN-γ, TNF-α and IL-1β. The results are shown in FIG. 1A and FIG. 1B.

As shown in FIG. 1A, a reduction in the inductions of IFN-γ was observed in the plasma of animals 6 hours following dosing with 50 μg of DNA-LNP together with 50 μg large ODN, compared with that of the dosing with 50 μg of DNA-LNP alone.

As shown in FIG. 1B, a reduction in the inductions of IFN-γ was not observed in any of the animals following dosing with 10 μg of DNA-LNP.

### Transgene Expression

On days 1, 8, 14, 28, 63, 84, and 98, plasma samples were analyzed using ELISA for circulating hFIX levels.

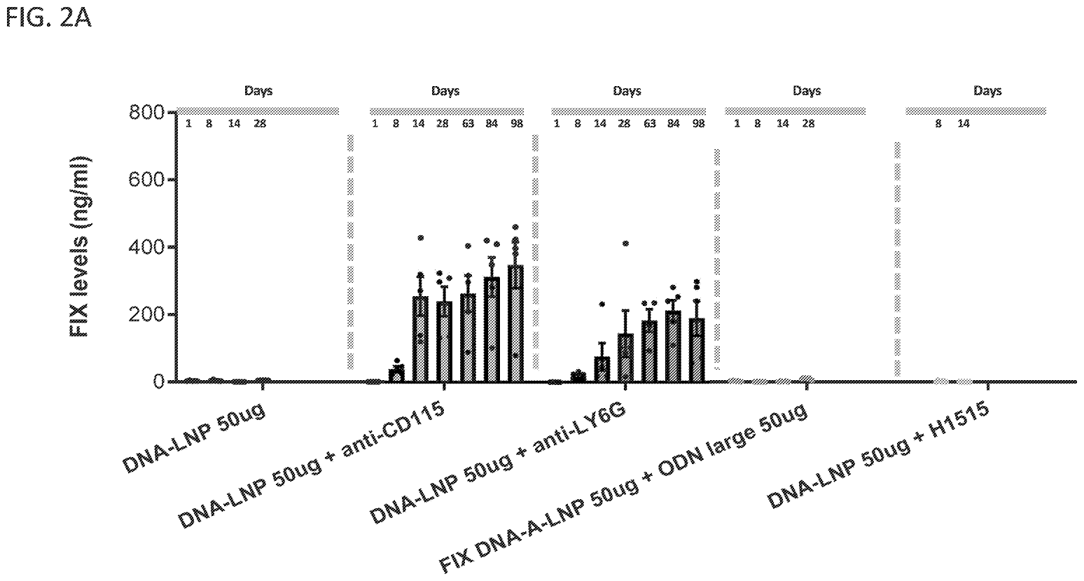

As shown in FIG. 2A, essentially no FIX expression was detected in the plasma of animals administered with 50 μg of DNA-LNP, without anti-CD115 or anti-Ly6G antibodies, while FIX expression of about 250 ng/mL (about 5% or more of normal expression level) was observed in the plasma of animals for over 14 weeks following dosing with 50 μg of DNA-LNP in combination with an anti-CD115 antibody, and FIX expression of about 100-200 ng/mL (less than 5% normal expression level) was observed in the plasma for over 14 weeks following dosing with 50 μg of DNA-LNP in combination with an anti-Ly6G antibody.

As shown in FIG. 2B, essentially no FIX expression was detected in the plasma of animals administered with 10 μg of DNA-LNP, without anti-CD115 or anti-Ly6G antibodies, while FIX expression of about 250 ng/mL (about 5% or more of normal expression level) was observed in the plasma of animals for over 14 weeks following dosing with 10 μg of DNA-LNP in combination with an anti-CD115 antibody, and FIX expression of about 100-300 ng/mL was observed in the plasma for over 14 weeks following dosing with 10 μg of DNA-LNP in combination with an anti-Ly6G antibody.

Taken together, the results demonstrate that immune cell depletion with an anti-CD115 improved transgene expression with a decreased immune response.

### Example 2—Immune Cell Depletion with Anti-CD115 and Another Immunosuppressant Improved Transgene Expression and Reduced Cytokine Production

The effects of immune cell depletion on cytokine release and on transgene expression were studied using an anti-CD115 antibody in combination with clodronate, which is a toxic agent encapsulated in liposomes to induce monocyte/macrophage apoptosis.

FIX transgene DNA plasmid (50 μg), formulated in lipid nanoparticle (LNP) delivery vehicles (SPK-LNP1), was dosed in mice (n=5; Balb/c; Jackson Laboratory) by intravenous administration alone, or in combination with anti-CD115 antibody and/or clodronate (purchased from Liposoma), as outlined in Table 3. SPK-LNP-1 had the following composition: C12-200 lipid at 35%; DOPE at 16%; cholesterol at 46.5%; and C14-PEG2000 (Avanti® Polar Lipids) at 2.5%. DNA and anti-CD115 antibody were as described in Example 1.

TABLE 3
Experimental Design

| Group | Group name | Day −5 | Day −2 | Day −1 | Hour 0 | Day 2 |
|---|---|---|---|---|---|---|
| 1 | SPK-LNP1 (50 μg) | | | | SPK-LNP1 (50 μg) | |
| 2 | SPK-LNP1 (50 μg) + | | | 1250 μg | SPK-LNP1 (50 μg) | |
| | 1250 μg Clodronate (IV) | | | Clodronate | | |
| | | | | (IV) | | |
| 3 | SPK-LNP1 | 300 μg | 300 μg | | SPK-LNP1 | 300 μg |
| | (50 μg) + 300 μg | Anti-CD115 | Anti-CD115 | | (50 μg) + | Anti-CD115 |
| | Anti-CD115 (IP) | (IP) | (IP) | | 300 μg | (IP) |
| | | | | | Anti-CD115 (IP) | |
| 4 | SPK-LNP1 (50 μg) + | 300 μg | 300 μg | 1250 μg | SPK-LNP1 | 300 μg |
| | 1250 μg Clodronate (IV) + | Anti-CD115 | Anti-CD115 | Clodronate | (50 μg) + | Anti-CD115 |
| | 300 μg Anti-CD115 (IP) | (IP) | (IP) | (IV) | 300 μg | (IP) |
| | | | | | Anti-CD115 (IP) | |

### Immune Response

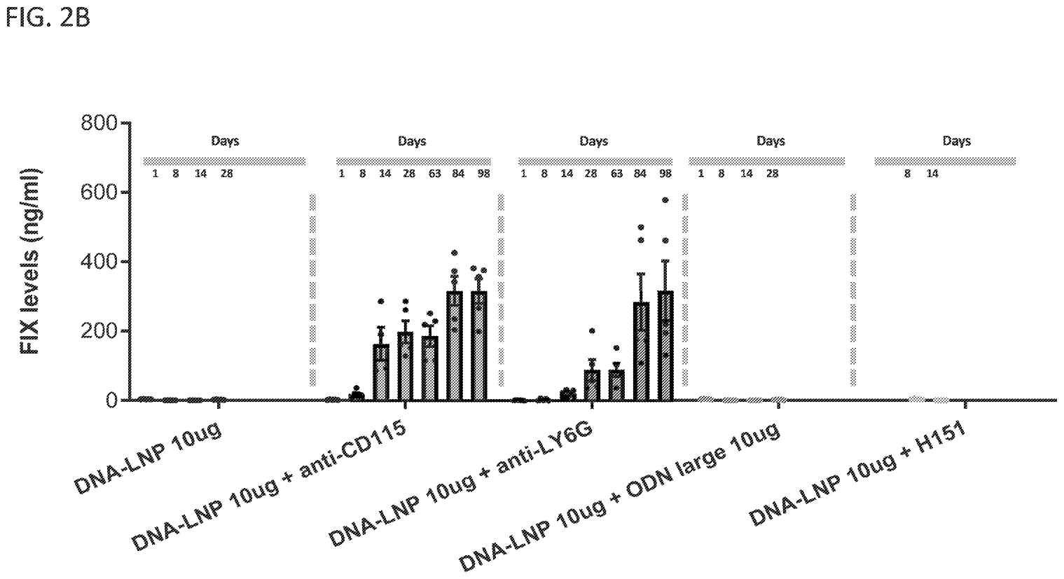

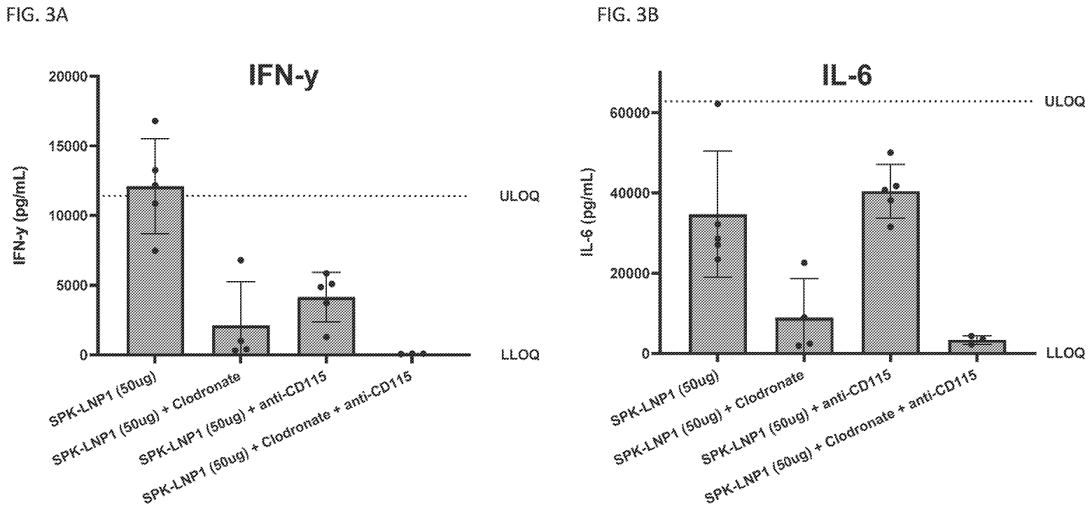

6-hour post-dose plasma samples were analyzed for a panel of pro-inflammatory cytokines using MSD: IL-6, IFN-γ, TNF-α and IL-1β. As shown in FIGS. 3A and 3B, immune cell depletion with anti-CD115 antibody or clodronate alone moderately reduced cytokine (INF-γ and/or IL-6) levels following DNA-LNP administration, and combining anti-CD115 antibody and clodronate treatments nearly abrogated cytokine inductions.

### Transgene Expression

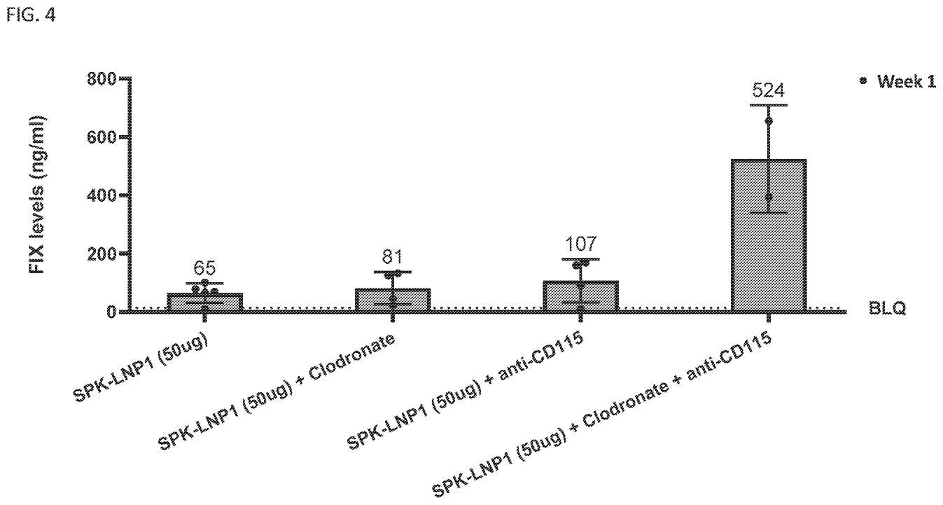

On week 1, plasma samples were analyzed using ELISA for circulating hFIX levels. As shown in FIG. 4, FIX expression of about 500 ng/mL (-10% of normal expression levels) was observed in the plasma of animals for one week following dosing with 50 μg of SPK-LNP1 in combination with an anti-CD115 antibody and clodronate, which is significantly higher than that when SPK-LNP1 was administered alone, or in combination with only one of the anti-CD115 antibody and clodronate.

Taken together, the results demonstrate that the double regimen of anti-CD115 antibody and clodronate nearly abrogated the immune responses while improving transgene expression.

### Example 3—Immune Cell Depletion with Clodronate, Anti-CD115 and Pexidartinib

Balb/C mice were treated with the immune cell depletion agents clodronate, anti-CD115 antibody (as described in Example 1), or pexidartinib. Clodronate was provided by IV administration at 50 mpk (1.25 mg) or 17 mpk (425 μg), anti-CD115 antibody was provided by IP administration at 12 mpk (300 μg) 1 dose vs 4 doses. Anti-CD115 antibody was as described in Example 1. Pexidartinib was provided by oral gavage administration at 100 mpk (2.5 mg) 4 doses. A no-treatment mice group was used as a control. Each group contained 5 male BALB/c mice.

After the treatment, mice were euthanized and the liver samples were harvested for histology. Liver samples were stained with either anti-CLEC4F or anti-CD68 mouse antibody. Individual scores of the treatment groups were normalized by the no-treatment group to calculate depletion efficacy. The specific treatment dose levels and schedules are shown in Table 4:

TABLE 4

| Group | | | | | | | |
|---|---|---|---|---|---|---|---|
| Number | Group Name | day −8 | day −6 | day −4 | day −2 | day −1 | day 0 |
| 1 | No treatment | | | | | | Histology |
| 2 | 50 mpk | | | | | Clodronate | Histology |
| | Clodronate | | | | | (50 mpk) | |
| 3 | 17 mpk | | | | | Clodronate | Histology |
| | Clodronate | | | | | (17 mpk) | |
| 4 | anti-CD115 | | | | anti-CD115 | | Histology |
| | (1 dose) | | | | | | |
| 5 | anti-CD115 | anti-CD115 | anti-CD115 | anti-CD115 | anti-CD115 | | Histology |
| | (4 doses) | | | | | | |
| 6 | Pexidartinib | Pexidartinib | Pexidartinib | Pexidartinib | Pexidartinib | | Histology |

The results are shown in FIGS. 5A and 5B. Clodronate and anti-CD115 antibodies had a significant effect on Kuffer and macrophage depletion. Pexidartinib had a small impact on Kupffer cell depletion (CLEC4F+ staining) and no impact on CD68+ macrophage depletion. The results with pexidartinib were attributed to the mode of administration.

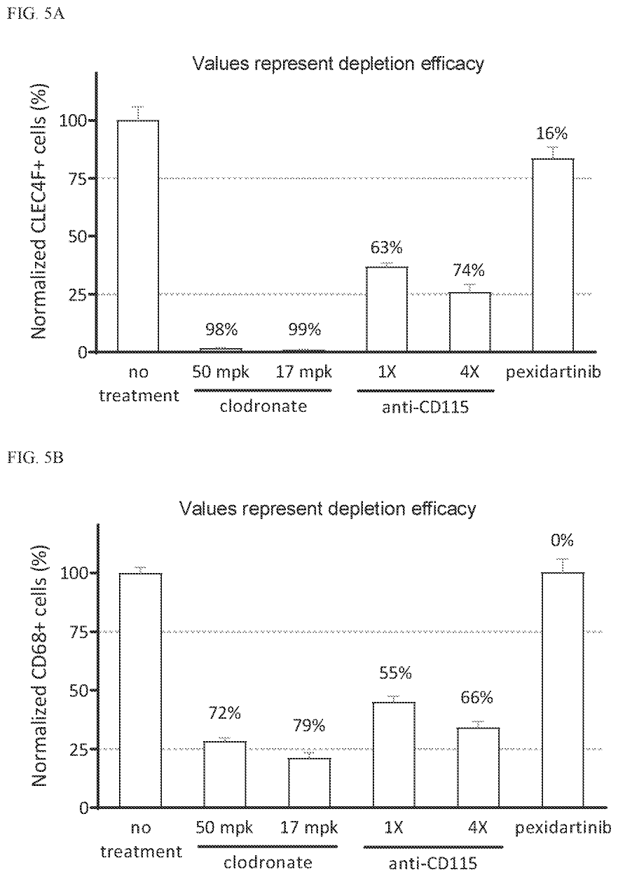

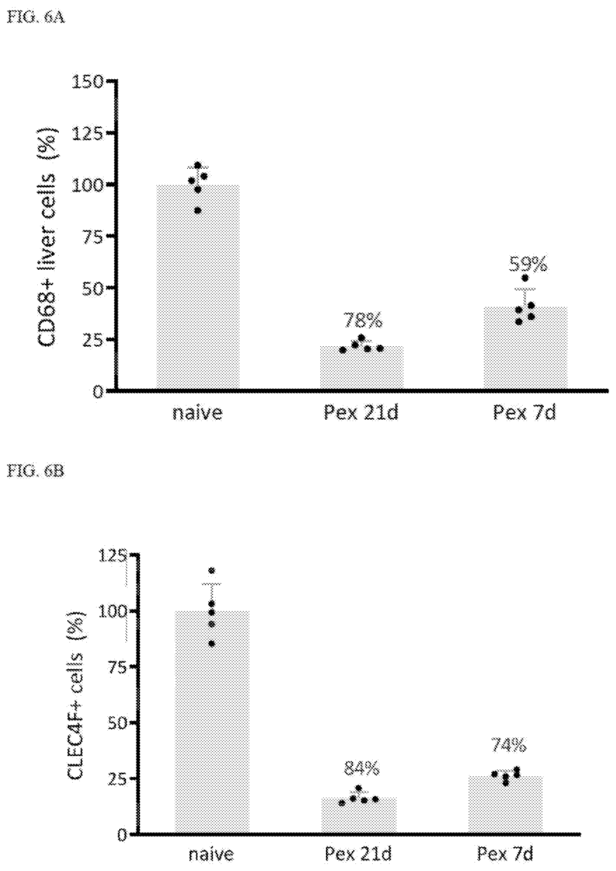

Administering pexidartinib mixed with mice chow provided for significant depletion of CLEC4F+ Kupffer cells and CD68+ macrophages. Pexidartinib was mixed with mice chow at 400 mg Pex/kg chow. Liver histology was taken after mice were fed with Pex-chow for 7 or 21 days. The results are shown in FIG. 6A and FIG. 6B.

### Example 4—Transgene Expression DNA-LNP-5 after Pexidartinib Treatment

Pexidartinib was mixed with mice chow at 400 mg Pex/kg chow. In group 2 and group 3, mice chow was replaced with Pex chow at 21 days and 7 days before DNA-LNP-5 dosing. The experimental design is shown in Table 5.

TABLE 5

| Group # | Group name | LNP | day −21 | day −7 | 0 hr | wks 1-4 |
|---|---|---|---|---|---|---|
| 1 | No depletion | DNA-LNP-5 | — | — | DNA-LNP-5 | Bleed |
| | | | | | Dosing | |
| 2 | 21-day Pexidartinib | | Start Pexidartinib | | DNA-LNP-5 | Bleed |
| | treatment | | chow | | Dosing | |
| 3 | 7-day Pexidartinib | | — | Start Pexidartinib | DNA-LNP-5 | Bleed |
| | treatment | | | chow | Dosing | |

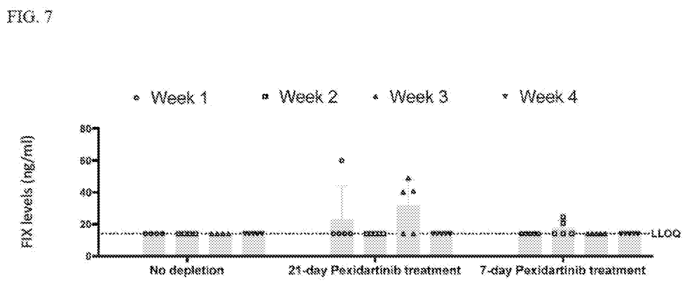

Mice in group 1 were fed with regular chow throughout the study and served as controls. Mice were dosed with DNA-LNP-5 (10 μg per mouse). LNP-5 (GenVoy™ LNP) was obtained from Precision NanoSystems Inc.) and contained: ionizable lipid at 50%; DSPC at 10%; cholesterol 37.5%; and stabilizer (PEG-Lipid) at 2.5% (see Roces et al., Pharmaceutics, 2020 12, 1095.) DNA was as described in Example 1. hFIX levels in mouse plasma were quantified using ELISA 1-4 weeks after dosing. The results are shown in FIG. 7. After DNA-LNP-5 dosing, a few mice in Pex-treated groups showed detectable expression. By contrast, no detectable hFIX levels was observed in mice fed with regular chow.

### Example 5—Transgene Expression DNA-LNP-5 after Anti-CD115 Treatment

Mice were treated with anti-CD115 antibody (300 μg anti-CD115 per injection) three times (days −5, −3, and −1) before DNA-LNP-5 dosing (10 μg per mouse). Mice in the control group were treated with DNA-LNP-5 only. DNA and anti-CD115 antibody were as described in Example 1. The experimental design is shown in Table 6.

TABLE 6

| | | | | | | | wks 1, |
|---|---|---|---|---|---|---|---|
| Group # | Group name | LNP | day −5 | day −3 | day −1 | day 0 | 2, 4, 12 |
| 1 | DNA-LNP-5 | DNA-LNP-5 | — | — | — | DNA-LNP-5 Dosing | Bleed |
| 2 | DNA-LNP-5 + | | Anti-CD115 | Anti-CD115 | Anti-CD115 | DNA-LNP-5 Dosing | Bleed |
| | anti-CD115 | | | | | | |

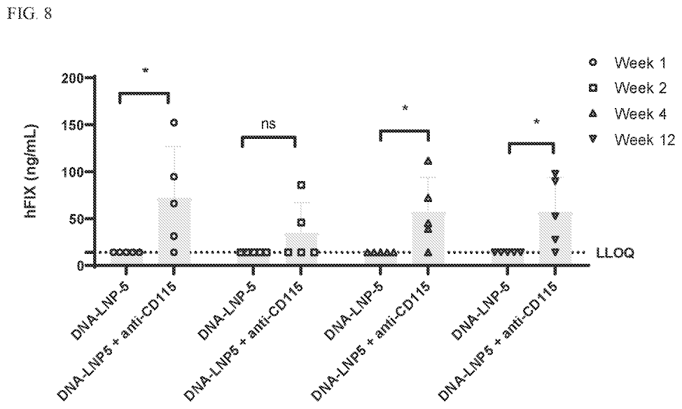

After DNA-LNP-5 dosing, hFIX levels in mouse plasma were quantified using ELISA. The result are shown in FIG. 8, where “ns” denotes not significant and *P<0.05 by t-test.

### Example 6—Transgene Expression DNA-LNP-3 after Anti-CD115 Treatment

Mice were treated with anti-CD115 antibody (300 μg anti-CD115 per injection) four times (days −10, −8, −6, and −2) before DNA-LNP-3 dosing (25 μg per mouse). Mice in the control group were treated with DNA-LNP-3 only. DNA-LNP-3 had the following composition: cKK-E12 at 35%; DOPE at 16%; cholesterol at 46.5%; and PEGylated lipid C14-PEG2000 at 2.5%. DNA and anti-CD115 antibody were as described in Example 1.

The experimental design is shown in Table 7.

TABLE 7

| | | | | | | | | wks 1, |
|---|---|---|---|---|---|---|---|---|
| Group # | Group name | LNP | day −10 | day −8 | day −6 | day −2 | day 0 | 2, 4, 12 |
| 1 | DNA-LNP-3 | DNA-LNP-3 | — | — | — | — | DNA-LNP-3 | Bleed |
| | | | | | | | Dosing | |
| 2 | DNA-LNP-3 + | | Anti- | Anti- | Anti- | Anti- | DNA-LNP-3 | Bleed |
| | anti-CD115 | | CD115 | CD115 | CD115 | CD115 | Dosing | |

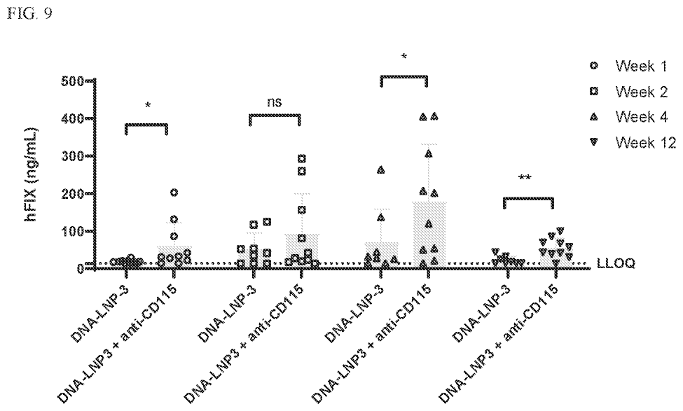

After DNA-LNP-3 dosing, hFIX levels in mouse plasma were quantified using ELISA. The results are shown in FIG. 9, where “ns” denotes not significant, *P<0.05, **P<0.01, and ***P<0.001 by t-test.

### Transgene Expression DNA-LNP-4 after Anti-CD115 Treatment

Mice were treated with anti-CD115 antibody (300 μg anti-CD115 per injection) four times (days −10, −8, −6, and −2) before DNA-LNP-4 dosing. Mice in the control group was treated with DNA-LNP-4 only. LNP-4 had the following composition: Lipid 9 (Sabnis et al., Molecular Therapy 2018, vol. 26, No. 6, 1509-1519) at 50%; DSPC at 10%; cholesterol at 38.5%; and C-14-PEG2000 at 1.5%. The experimental design is shown in Table 8.

TABLE 8

| | | | | | | | | wks 1, |
|---|---|---|---|---|---|---|---|---|
| Group # | Group name | LNP | day −10 | day −8 | day −6 | day −2 | day 0 | 2, 4, 12 |
| 1 | DNA-LNP-4 | DNA-LNP-4 | — | — | — | — | DNA-LNP-4 | Bleed |
| | | | | | | | Dosing | |
| 2 | DNA-LNP-4 + | | Anti- | Anti- | Anti- | Anti- | DNA-LNP-4 | Bleed |
| | anti-CD115 | | CD115 | CD115 | CD115 | CD115 | Dosing | |

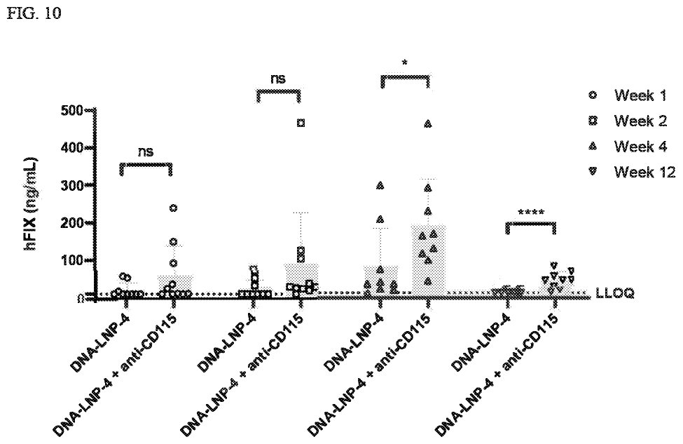

After DNA-LNP-4 dosing, hFIX levels in mouse plasma were quantified using ELISA. The results are shown in FIG. 10, where “ns” denotes not significant, *P<0.05 and ***P<0.001 by t-test.

The instant invention is generally disclosed herein using affirmative language to describe the numerous embodiments of the instant invention. The instant invention also specifically includes embodiments in which particular subject matter is excluded, in full or in part, such as substances or materials, method steps and conditions, protocols, or procedures. For example, in certain embodiments of the instant invention, materials and/or method steps are excluded. Thus, even though the instant invention is generally not expressed herein in terms of what the instant invention does not include, aspects that are not expressly excluded in the instant invention are nevertheless disclosed herein.

I/we claim:

1. A method of delivering a transgene to a subject in need thereof comprising:
a. administering to the subject a phagocyte-depleting agent; and
b. administering to the subject a pharmaceutical composition comprising a non-viral vector comprising the transgene and a pharmaceutically acceptable carrier.

2. The method of claim 1, wherein the transgene is expressed in the subject.

3. The method of any one of claims 1-2, wherein the transgene is operably linked to a promoter or promoter/enhancer.

4. The method of any one of claims 1-3, wherein the transgene is operably linked to a liver specific promoter or liver specific promoter/enhancer.

5. The method of claim 4, wherein said liver specific promoter or liver specific promoter/enhancer is selected from the group consisting of: transthyretin (TTR) gene promoter and mutant versions thereof, human alpha 1-antitrypsin (hAAT) promoter, apolipoprotein A-I promoter, albumin promoter, hepatitis B virus core promoter, alpha-fetoprotein (AFP), human Factor IX promoter, thyroxin binding globulin (TBG) promoter, and apolipoprotein E (ApoE)/hAAT.

6. The method of any one of claims 1-5, wherein the non-viral vector is a non-viral delivery nanoparticle.

7. The method of any one of claims 1-6, wherein the non-viral vector and the phagocyte-depleting agent are co-administered.

8. The method of any one of claims 1-6, wherein the phagocyte-depleting agent is administered at least one day before the non-viral vector is administered.

9. The method of any one of claims 1-8, wherein the phagocyte-depleting agent is a CD115-inhibiting agent.

10. The method of claim 9, wherein the CD115-inhibiting agent is an antibody or an antigen binding fragment thereof that specifically binds to CD115.

11. The method of claim 10, wherein the antibody or an antigen binding fragment thereof is emactuzumab, AMG820, or cabiralizumab or an antigen-binding fragment thereof.

12. The method of any one of claims 1-11, further comprising administering to the subject a bisphosphonate.

13. The method of claim 12, wherein the bisphosphonate is clodronate, pamidronate, ibandronate, or zoledronate.

14. The method of claim 13, wherein the bisphosphonate is clodronate.

15. The method of any one of claims 1-14, further comprising administering to the subject an immunosuppressant.

16. The method of claim 15, wherein the immunosuppressant is a steroid.

17. The method of claim 16, wherein the steroid is a corticosteroid.

18. The method of claim 17, wherein the corticosteroid is dexamethasone.

19. The method of any one of claims 1-18, wherein the subject has a minimal or absent undesirable immune response induced by the non-viral vector.

20. The method of any one of claims 6-19, wherein the non-viral delivery particle is selected from the group consisting of a lipid nanoparticle, a polymer nanoparticle, a protein-based nanoparticle, and a peptide cage.

21. The method of any one of claims 1-20, wherein the non-viral vector is a dsDNA molecule, and wherein the dsDNA molecule is selected from the group consisting of a minicircle, a plasmid, open linear duplex DNA, and a closed-ended linear duplex DNA (CELiD/ceDNA/doggybone DNA).

22. The method of any one of claims 1-20, wherein the non-viral vector is an ssDNA molecule, and wherein the ssDNA molecule is selected from the group consisting of a closed circular DNA and an open linear DNA.

23. The method of any one of claims 1-22, wherein the transgene encodes a therapeutic or prophylactic protein or peptide.

24. The method of any one of claims 1-22, wherein the transgene encodes a therapeutic or prophylactic nucleic acid.

25. The method of any one of claims 6-24, wherein the non-viral delivery nanoparticle is a lipid nanoparticle (LNP) comprising one or more lipids as provided in Table 1, or a pharmaceutically acceptable salt thereof.

26. The method of claim 25, wherein said one or more lipids are present in the LNP at a mol% of about 20% to about 65%.

27. A kit comprising:
a. a pharmaceutical composition comprising a non-viral vector comprising a transgene and a pharmaceutically acceptable carrier; and
b. a phagocyte-depleting agent.

28. The kit of claim 27, wherein the non-viral vector is a non-viral delivery nanoparticle and the transgene is operably linked to a promoter or a promoter/enhancer.

29. The kit of claim 27 or 28, further comprising at least one of a bisphosphonate, a corticosteroid, and an immunosuppressant.

30. The kit of claim 28 or 29, wherein the phagocyte-depleting agent is a CD115-inhibiting agent and the kit comprises:
a. the pharmaceutical composition comprising the non-viral delivery nanoparticle and the non-viral vector;
b. the CD115-inhibiting agent; and
c. at least one of clodronate, dexamethasone, and a JAK inhibitor.

31. The kit of claim 30, wherein the CD115-inhibiting agent is emactuzumab, AMG820, or cabiralizumab or an antigen-binding fragment thereof.
